(19) United States
(12) Patent Application Publication
(10) Pub. No.: US 2023/0022590 A1
(43) Pub. Date: Jan. 26, 2023

(54) NOVEL RNA THERAPEUTICS AND USES THEREOF

(71) Applicant: Eli Lilly and Company, Indianapolis, IN (US)

(72) Inventors: Rebecca Ruth MILES, Pendleton, IN (US); Jibo WANG, Carmel, IN (US); Melissa Ann BELLINGER, Zionsville, IN (US); Thomas Patrick BEYER, Zionsville, IN (US); Christine Chih-Tao CHENG, Carmel, IN (US); MariJean EGGEN, Brownsburg, IN (US); Gregory Lawrence LACKNER, Indianapolis, IN (US)

(21) Appl. No.: 17/846,636

(22) Filed: Jun. 22, 2022

Related U.S. Application Data
(60) Provisional application No. 63/214,584, filed on Jun. 24, 2021.
(60) Provisional application No. 63/214,555, filed on Jun. 24, 2021.

(51) Int. Cl. A61K 47/54; C12N 15/113

(52) U.S. Cl. CPC A61K 47/549; A61K 47/545; C12N 15/113; C12N 2310/14; C12N 2310/351; C12N 2320/32

(57) ABSTRACT

The present disclosure relates to novel RNAi agents designed to decrease the expression of ANGPTL8 in the liver, where the RNAi agents comprise delivery moieties conjugated to oligonucleotides optionally via a linker. The RNAi agents are useful in the treatment of diseases involving the regulation of ANGPTL8 expression.

The present disclosure relates to novel RNAi agents designed to decrease the expression of ANGPTL8 in the liver, where the RNAi agents comprise delivery moieties conjugated to oligonucleotides optionally via a linker. The RNAi agents are useful in the treatment of diseases involving the regulation of ANGPTL8 expression.

Angiopoietin-like protein 8 (ANGPTL8) is mainly expressed in liver and adipose tissue and it plays an important role in triglyceride metabolism. ANGPTL8, together with ANGPTL3 or ANGPTL4, is thought to regulate triglyceride levels by inhibiting the enzymatic activity of lipoprotein lipase (LPL), which, when active, hydrolyzes triglycerides and decreases circulating plasma triglycerides. Increased levels of ANGPTL8 are observed or associated with cardiovascular disease, diabetes, dyslipidemia (including high triglyceride levels), aberrant renal function, hypertension, nonalcoholic fatty liver disease such as nonalcoholic steatohepatitis (NASH), and obesity.

The RNAi agents, such as those disclosed herein, permit targeting genes in a sequence-specific manner for personalized treatment of many different types of diseases involving gene dysregulation. Compounds comprising oligonucleotides, such as the RNAi agents herein, can work via different mechanisms depending on the particular type of oligonucleotides employed. RNA interference molecules, including the RNAi agents disclosed herein, typically operate to knock down, or decrease, gene expression of a given target transcript, thereby decreasing the level of protein. By delivering RNAi molecules, such as the RNAi agents herein, to a desired tissue of the patient, gene expression can be decreased in a tissue specific manner.

RNAi agents comprising N-acetylgalactose (GalNAc) to target the asialoglycoprotein receptor on liver cells are one example. Specifically, givosiran is an FDA approved siRNA that targets ALAS1 gene transcript to treat acute hepatic porphyria, employs a delivery moiety comprising GalNAc for entry into liver cells. Insclisiran is an FDA approved siRNA that targets the PCSK9 gene transcript to lower LDL cholesterol, and also employs a delivery moiety comprising GalNAc for entry into liver cells. RNAi molecules comprising siRNAs targeting ANGPTL8 have been described, e.g., WO2020104649. However, only four siRNA molecules are approved for use in humans, and no therapeutic siRNAs targeting ANGPTL8 are yet approved. Moreover, limited information is available preclinically and clinically about the ideal attributes for a therapeutic siRNA in vivo, especially for diseases of the liver or involving the liver such as cardiovascular disease, dyslipidemia, e.g. high triglycerides, and inflammatory liver diseases.

There remains a need to provide alternative RNAi agents comprising a delivery moiety comprising GalNAc and one or more oligonucleotides to decrease ANGPTL8 expression. More particularly, there is a need to provide RNAi agents comprising a novel GalNAc delivery moiety and a sense strand and an antisense strand, wherein the antisense strand is complementary to ANGPTL8 mRNA, wherein such an RNAi agent exhibits one or more of: improved tissue exposure, suitably improved exposure in the liver; improved liver to kidney exposure ratios; improved knockdown; an improved durable response; an improved pharmacokinetic profile; fewer off target effects; an improved toxicity profile; an improved safety profile, fewer side effects, improved tolerability, improved control of cholesterol and/or triglyceride levels in a patient, improved cardiovascular risk profile in a patient, improved and/or simplified synthesis, synthetic processes with fewer degradation products, or any combination thereof.

In one embodiment of the present disclosure is an RNA interference (RNAi) agent comprising a delivery moiety of Formula I:

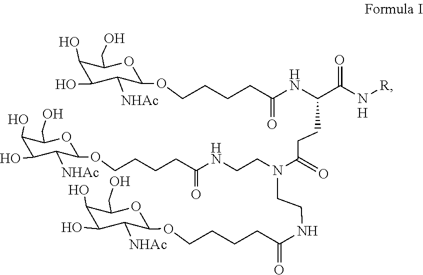

wherein R comprises a sense strand and an antisense strand, and wherein the antisense strand comprises at least 15 contiguous nucleotides of a sequence that is complementary to the mRNA transcript of ANGPTL8, and wherein the sense strand and the antisense strand form a region of complementarity of at least 15 nucleotides, and wherein the sense strand and antisense strand are each independently 15 to 30 nucleotides in length, and optionally wherein the sense strand and antisense strand each independently comprise one or more modified nucleotides, and optionally wherein the sense strand and the antisense strand each independently comprise one or more modified internucleotide linkages, and wherein R is optionally conjugated to Formula I via a linker. In another embodiment, the antisense strand comprises at least 15 contiguous nucleotides of a sequence that is complementary to SEQ ID NO:1. In a further embodiment, the sense strand and antisense strand are each independently 18 to 23 nucleotides in length. In a further embodiment of any of these RNAi agents, the antisense strand forms a region of complementarity of at least 18 nucleotides to the mRNA transcript of ANGPTL8. In a different further embodiment of any of these RNAi agents, the antisense strand forms a region of complementarity of at least 18 nucleotides to SEQ ID NO:1.

In a further embodiment is an RNAi agent wherein the antisense strand comprises at least 15 nucleotides of a sequence selected from the group consisting of SEQ ID NO:s 405-525. In yet a further embodiment, the antisense strand comprises at least 18 contiguous nucleotides of a sequence selected from the group consisting of SEQ ID NOs: 405-525.

In a further embodiment of the RNAi agents disclosed herein, the antisense strand comprises at least 18 contiguous nucleotides of a sequence selected from the group consisting of SEQ ID NOs: 405, 408, 412, 413, 414, 415, 418, 420, 425, 426, 428, 429, 432, 433, 434, 435, 436, 437, 438, 439, 440, 441, 442, 443, 444, 445, 446, 448, 449, 451, 452, 454, 457, 458, 459, 463, 464, 465, 466, 467, 468, 469, 471, 472, 473, 474, 475, 476, 479, 490, 491, 492, 493, 495, 499, 500, 501,502, 503, 504, 505, 506, 507, 508, and 509. In another embodiment, the antisense strand is selected from the group of antisense strand sequences in Table 2.

In any of the embodiments of the RNAi agents disclosed herein, the RNAi agent of any the anti sense strand is 23 nucleotides in length, or the sense strand is 21 nucleotides in length, or both.

In another embodiment of the RNAi agents disclosed herein, the antisense strand is selected from the group consisting of SEQ ID NOs: 231-361, or a sequence having at least 90% sequence identity thereto. In other embodiments of the RNAi agents disclosed herein, the sense strand is selected from the group consisting of SEQ ID NOs: 124-230, or a sequence having at least 90% sequence identity thereto.

In further embodiments of any of the RNAi agents described herein, the region of complementarity comprises 0, 1, 2, or 3 mismatches between the sense strand and the antisense strand.

In further embodiments of the RNAi agents, the sense strand and the antisense strand each independently comprise one or more modified nucleotides. In a further embodiment, the one or more modified nucleotides are independently 2′ fluoro modified nucleotides or 2′-O-methyl modified nucleotides. In other embodiments, each nucleotide of the sense strand and each nucleotide of the antisense strand is a modified nucleotide, and in further embodiment, each of the modified nucleotides are independently a 2′ fluoro modified nucleotide or a 2′-O-methyl modified nucleotide.

In other embodiments of the RNAi agents herein, the sense strand and antisense strand each independently comprise one or more modified internucleotide linkages. In a further embodiment, each modified internucleotide linkage is a phosphorothioate linkage. In other embodiments, the sense strand and antisense strand each independently comprise four phosphorothioate linkages. In further embodiments, the first two 5′ nucleotides of the sense strand and the two terminal 3′ nucleotides of the sense strand are phophorothioate linkages. In further embodiments, the first two 5′ nucleotides of the antisense strand and the two terminal 3′ nucleotides of the antisense strand are phosphorothioate linkages.

In other embodiments of the RNAi agents herein, the 5′ nucleotide of the antisense strand comprises a phosphate group or a phosphate analog.

In another embodiment, the present disclosure provides an RNAi agent comprising a delivery moiety of Formula I conjugated to R:

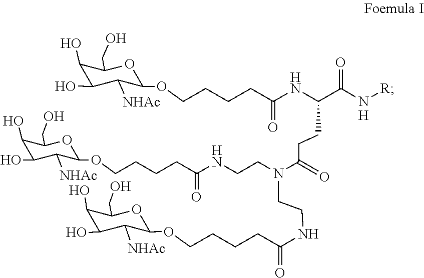

wherein R comprises a sense strand and an antisense strand, and wherein the antisense strand comprises at least 15 contiguous nucleotides of a sequence that is complementary to the mRNA transcript of ANGPTL8, and wherein the sense strand and the antisense strand form a region of complementarity of at least 15 nucleotides, and wherein the sense strand and antisense strand are each independently 15 to 30 nucleotides in length, and optionally wherein the sense strand and antisense strand each independently comprise one or more modified nucleotides, and optionally wherein the sense strand and the antisense strand each independently comprise one or more modified internucleotide linkages, and wherein R is optionally conjugated to a delivery moiety, D, of Formula I via a linker, L. In another embodiment, the antisense strand comprises at least 15 contiguous nucleotides of a sequence that is complementary to SEQ ID NO:1. In a further embodiment, the sense strand and antisense strand are each independently 18 to 23 nucleotides in length. In a further embodiment of any of these RNAi agents, the antisense strand forms a region of complementarity of at least 18 nucleotides to the mRNA transcript of ANGPTL8. In a different further embodiment of any of these RNAi agents, the antisense strand forms a region of complementarity of at least 18 nucleotides to SEQ ID NO:1. In further embodiments, wherein the antisense strand comprises at least 15 contiguous nucleotides of a sequence selected from the group consisting of SEQ ID NOs: 405-525.

Any of the compounds herein, including the RNAi agents disclosed herein, comprising Formula I may have modifications or additions within Formula I, or the compounds may comprise additional moieties. For example, one or more alkyl chains in Formula I may be extended or shortened, or the compound comprising Formula I may further comprise one or more oligonucleotides. The compounds herein comprising Formula I, such as the RNAi agents disclosed herein, are useful for, e.g. delivering one or more oligonucleotides, to a cell that has a receptor for one or more N-acetylgalactose (GalNAc, also N-GalNAc or galnac or Galnac) moieties, such as the asialoglycoprotein receptor (ASPGR) that typically binds three GalNAc moieties. Accordingly, the compounds comprising Formula I herein, such as the RNAi agents disclosed herein, may be used to preferentially bind to liver cells that express ASPGR, thereby facilitating entry of the compounds into liver cells. As ASPGR is also present on adipose tissue, the compounds comprising Formula I, including the RNAi agents herein thus may be used to deliver oligonucleotides to fat cells that express ASPGR.

In one embodiment is a compound or RNAi agent comprising a delivery moiety and one or more oligonucleotides, wherein the delivery moiety comprises Formula I, and wherein the oligonucleotides are complementary to the ANGPTL8 gene (hereinafter ANGPTL8 oligonucleotides). In a suitable further embodiment, the delivery moiety comprising Formula I delivers the one or more ANGPTL8 oligonucleotides to liver tissue, by binding to the extracellular receptor ASPGR and permitting entry of the oligonucleotides into the cells that comprise the liver tissue. The ANGPTL8 oligonucleotides are also represented herein by R or a sense strand and/or antisense strand herein, including as set forth in the sense and antisense sequences as set forth in the SEQ IDs herein.

The delivery moiety comprising Formula I can be used to deliver any number of ANGPTL8 oligonucleotides, such as the RNAi agents comprising R, including wherein R comprises a sense strand and/or an antisense strand disclosed herein, for diagnostic or, suitably, for therapeutic purposes. The one or more oligonucleotides such as the sense strands and antisense strands disclosed herein may comprise DNA or RNA nucleotides or DNA or RNA nucleosides.

The oligonucleotides herein, including the antisense strands herein, are designed to target, that is, bind or anneal to, or form a regions of complementarity with, ANGPTL8 sequences in a cell to regulate gene expression, suitably decreasing ANGPTL8 gene expression. In one embodiment is a compound or RNAi agent comprising Formula I disclosed herein, for decreasing expression of an ANGPTL8 transcript. In a further embodiment the compound or RNAi agent comprising Formula I disclosed herein for decreasing expression of an ANGPTL8 transcript further decreases ANGPTL8 protein expression. In another embodiment, the decrease in expression of a target transcript or target protein is about 99, 98, 97, 96, 95, 94, 93, 92, 91, 90, 85, 80, 75, 70, 65, 60, 55, or 50 percent. In a further embodiment, the decrease in expression is durable for about three weeks, about one month, about one and half months, about two months, about three months, about four months, about five months, or about six months.

One of skill in the art recognizes that one or more mismatches may be present as between the ANGPTL8 oligonucleotide, such as an antisense nucleotide disclosed herein, and the ANGPTL8 target nucleotide sequence and still function to regulate gene expression. In another embodiment, the oligonucleotide has 99, 98, 97, 96, 95, 94, 93, 92, 91, 90, 89, 88, 87, 86, 85, 84, 83, 82, 81, 80, 79, 78, 77, 76, 75, 74, 73, 72, 71, or 70 percent sequence identity with the target sequence or complementary to the target sequence. The oligonucleotides may also have overhangs of 1-10, 1-5, or 1-3, or 3, 2, or 1 residue(s) at either the 5′ or 3′ end. The 5′ or 3′ ends may be further modified, for example with an abasic residue or a phosphate group.

The term “percent sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity, using the PID3 calculation, which is the number of identical nucleotide residues divided by the total number of nucleotides, nucleosides, or nucleobases of the shortest of the two sequences, multiplied by 100. See, e.g., Raghava, G., Barton, G. J. Quantification of the variation in percentage identity for protein sequence alignments. BMC Bioinformatics 7, 415 (2006). Those skilled in the art can determine appropriate parameters for measuring alignment, including any algorithms needed to achieve maximal alignment over the full length of the sequences being compared.

One of skill in the art recognizes that modifications of the RNAi agents (or compounds or RNAi molecules) comprising ANGPTL8 oligonucleotides, such as the sense strand or antisense strands of the RNAi agents disclosed herein, can increase its stability and half life. The modifications may be to the nucleotide or to the phosphodiester backbone, that is the bonds between two nucleotide residues of the oligonucleotide, which is also termed an internucleotide linkage. For example, 2′-modifications on the sugar residue, suitably ribose, can increase its stability and half-life. These modifications include, but are not limited to, a 2′ fluoro or 2′ methoxy modification at the 2′ OH group of an unmodified sugar. Backbone modifications, also termed modified internucleotide linkages herein, include a change from a phosphodiester bond to a phosphorothioate (PS) bond.

Additional embodiments of the RNAi agents comprising a delivery moiety of Formula I comprise such nucleotide and internucleotide linkage modifications. Accordingly, in one embodiment is an RNAi agent comprising a delivery moiety of Formula I:

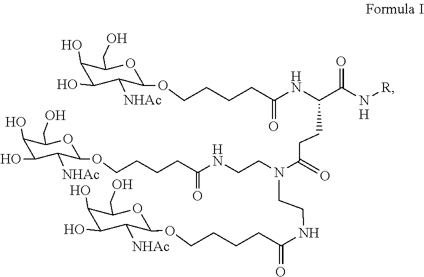

wherein R comprises a sense strand and an antisense strand, wherein the antisense strand comprises at least 15 contiguous nucleotides of a sequence selected from the group consisting of SEQ ID NOs: 405-525, and wherein the sense strand and the antisense strand form a region of complementarity of at least 15 nucleotides, and wherein the sense strand and antisense strand are each independently 18 to 23 nucleotides in length, and wherein the sense strand and antisense strand each independently comprise one or more modified nucleotides, and optionally wherein the sense strand and the antisense strand each independently comprise one or more modified internucleotide linkages, and wherein R is optionally conjugated to Formula I via a linker, and wherein the one or more modified nucleotides are independently 2′ fluoro modified nucleotides or 2′-O-methyl modified nucleotides. In further embodiments, each nucleotide of the sense strand and each nucleotide of the anti sense strand is a modified nucleotide. In other embodiments, the sense strand and antisense strand each independently comprise one or more modified internucleotide linkages. In further embodiments, each modified internucleotide linkage is a phosphorothioate linkage. In still further embodiments, the sense strand and antisense strand each independently comprise four phosphorothioate linkages. In further embodiments, the first two nucleotides at the 5′ end of the sense strand and the last two nucleotides at the 3′ end of the sense strand comprise phosphorothioate linkages, and the first two nucleotides at the 5′ end of the antisense strand and the last two nucleotides at the 3′ end of the antisense strand comprise phosphorothioate linkages. In any of the embodiments of the RNAi agents disclosed herein, the 5′ nucleotide of the antisense strand comprises a phosphate group or a phosphate analog.

As used herein, “oligonucleotide” (or “multimer” or “oligomer,” used herein interchangeably) as used herein, including the sense strands and antisense strands disclosed herein, means a chain of at least 10 nucleotide or nucleoside residues, and may comprise modified or unmodified bases, modified or unmodified sugars, and/or modified or unmodified bonds (also referred to herein interchangeably as the backbone or phosphodiester backbone or internucleotide linkage). The nucleotide residues may be connected by phosphodiester bonds or modified bonds, also called phosphodiester internucleotide linkages or modified internucleotide linkages herein. The nucleotide residues may be modified at one or more atoms in the nucleobase pyrimidine or purine ring, or at one or more atoms in the sugar residue, or at one or more atoms of the bond between the ring-sugar and nucleobase. Modifications may also be made at the 5′ or 3′ end of the oligonucleotide strand and such modified oligonucleotides or sense or antisense strands may referred to interchangeably as an oligonucleotide or sense or antisense strand herein, unless the context makes clear otherwise. Herein, nucleoside residues (i.e. nucleotides that lack one or more phosphate groups) may be referred to as modified nucleotides/nucleotide residues/nucleotide bases or simply nucleotides/nucleotide residues/nucleotide bases.

As used herein, “complementary” means a structural relationship between two nucleotides (e.g., on two opposing nucleic acids or on opposing regions of a single nucleic acid strand, e.g., a hairpin) that would be expected to allow the two nucleotides to form base pairs with one another in the canonical Watson-Crick pairing. For example, a purine nucleotide of one nucleic acid that is complementary to a pyrimidine nucleotide of an opposing nucleic acid are complementary to each other. For example, they are predicted to base pair together by forming hydrogen bonds with one another. Likewise, two antiparallel nucleic acids may have regions of multiple nucleotides that are complementary with each other to form regions of complementarity, such as the sense strands and antisense strands of the RNAi agents described herein.

As used herein, “region of complementarity” means a nucleotide sequence of a nucleic acid (e.g., a ds oligonucleotide) that is sufficiently complementary to an antiparallel nucleotide sequence to permit hybridization between the two sequences of nucleotides under appropriate hybridization conditions (e.g., in a phosphate buffer, in a cell, etc.). In some embodiments, an oligonucleotide herein includes a targeting sequence having a region of complementary to a mRNA target sequence.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than hydrogen at the 2′ position of the sugar, including modifications or substitutions in or of the nucleobase, sugar, or phosphate group.

As used herein, “double-stranded oligonucleotide” or “ds oligonucleotide” means an oligonucleotide that is substantially in a duplex form. The complementary base-pairing of duplex region(s) of a ds oligonucleotide can be formed between antiparallel sequences of nucleotides of covalently separate nucleic acid strands. Likewise, complementary base-pairing of duplex region(s) of a ds oligonucleotide can be formed between antiparallel sequences of nucleotides of nucleic acid strands that are covalently linked. Moreover, complementary base-pairing of duplex region(s) of a ds oligonucleotide can be formed from single nucleic acid strand that is folded (e.g., via a hairpin) to provide complementary antiparallel sequences of nucleotides that base pair together. A ds oligonucleotide can include two covalently separate nucleic acid strands that are fully duplexed with one another. However, a ds oligonucleotide can include two covalently separate nucleic acid strands that are partially duplexed (e.g., having overhangs at one or both ends). A ds oligonucleotide can include an antiparallel sequence of nucleotides that are partially complementary, and thus, may have one or more mismatches, which may include internal mismatches or end mismatches.

As used herein, “duplex” and “duplex region” in reference to nucleic acids (e.g., oligonucleotides), means a double stranded nucleic acid structure formed through complementary base pairing of two antiparallel sequences of nucleotides, whether formed by two covalently separate nucleic acid strands or by a single, folded strand (e.g., via a hairpin), and may be formed via annealing or hybridization under appropriate conditions.

As used herein, “linker” means a structure used to conjugate a nucleotide (e.g., oligonucleotide) to a delivery moiety. A linker can be “labile” or “cleavable” meaning a linker that can be cleaved (e.g., by acidic pH). Likewise, a linker can be “stable” or “non-cleavable” meaning a linker that is not cleavable under physiological conditions.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

As used herein, “nucleotide” means an organic compound having a nucleoside (a nucleobase such as, for example, adenine, cytosine, guanine, thymine, or uracil, and a pentose sugar such as, for example, ribose or 2′-deoxyribose) and a phosphate group. A “nucleotide” can serve as a monomeric unit of nucleic acid polymers such as deoxyribonucleic acid (DNA) and ribonucleic acid (RNA).

As used herein, “overhang” means a terminal nucleotide(s) resulting from one strand or region extending beyond the terminus of a complementary strand with which the one strand or region forms a duplex. An overhang may include one or more unpaired nucleotides extending from a duplex region at the 5′ terminus or 3′ terminus of a ds oligonucleotide. The overhang can be a 3′ or 5′ overhang on the antisense strand or sense strand of a ds oligonucleotides.

As used herein, “phosphate analog” or “phosphate mimic” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317.

Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

“Percent complementarity” is number of nucleotides, nucleosides, or nucleobases between two strands that exhibit the canonical pairing, divided by the total nucleotides, nucleosides, or nucleobases of the shortest of the two sequences, multiplied by 100.

As used herein, “ribonucleotide” means a nucleotide having a ribose as its pentose sugar, which contains a hydroxyl group at its 2′ position. A modified ribonucleotide, also referred to as a modified nucleotide herein, is a ribonucleotide having one or more modifications or substitutions of atoms other than hydroxyl at the 2′ position, including modifications or substitutions in or of the nucleobase, sugar, or phosphate group.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “reduced expression,” and with respect to a gene (e.g., ANGPTL8) means a decrease in the amount or level of RNA transcript (e.g., ANGPTL8 mRNA) or protein encoded by the gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide herein (e.g., an oligonucleotide having an antisense strand having a nucleotide sequence that is complementary to a nucleotide sequence including ANGPTL8 mRNA) may result in a decrease in the amount or level of mRNA, protein, and/or activity (e.g., via degradation of ANGPTL8 mRNA by the RNAi pathway) when compared to a cell that is not treated with the ds oligonucleotide. Similarly, and as used herein, “reducing expression” means an act that results in reduced expression of a gene (e.g., ANGPTL8). Specifically, and as used herein, “reduction of ANGPTL8 expression” means a decrease in the amount or level of ANGPTL8 mRNA, ANGPTL8 protein, and/or ANGPTL8 activity in a cell, a population of cells, a sample, or a subject when compared to an appropriate reference (e.g., a reference cell, population of cells, tissue, or subject).

In certain embodiments the one or more oligonucleotides comprise a small interfering RNA (siRNA), miRNA (microRNA), short hairpin RNA (shRNA), single guide RNA (sgRNA), or antisense oligonucleotide (ASO). In a suitable embodiment, the one or more ANGPTL8 oligonucleotides comprises siRNA. In another suitable embodiment is an RNAi agent comprising a sense strand and antisense strand. In another suitable embodiment the one or more ANGPTL8 oligonucleotides is an siRNA comprising a sense strand and an antisense strand.

In some embodiments, the compound further comprises a linker, for example for conjugating one or more ANGPTL8 oligonucleotides, such as R, including wherein R comprises a sense strand and an antisense strand to Formula I. In other embodiments, the RNAi agent disclosed herein comprises a linker conjugating a double stranded oligonucleotide comprising a sense strand or an antisense strand. In other embodiments, the RNAi agent comprises a sense strand conjugated to the delivery moiety of Formula I via a linker. Suitable linkers are known in the art. In one embodiment, the linker comprises an alkyl chain, suitably C₁₋₈. In a further embodiment, the linker is an alkyl chain, suitably C₁₋₈. In a further embodiment, the linker comprises Linker 1, as shown below (Formula II herein having connection points A and B). In a further embodiment, the linker is Linker 1. In another embodiment the linker comprises a piperidine ring. In a further suitable embodiment, the linker comprises Linker 2, as shown below (Formula III herein having connection points C and D). In a further suitable embodiment, the linker is Linker 2.

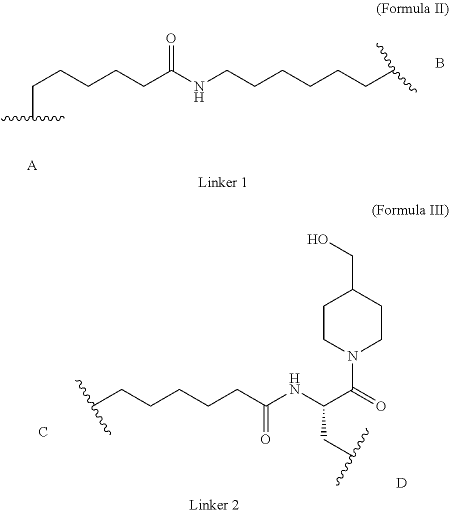

One of skill in the art will recognize that the linker may be on the 5′ or 3′ end of an ANGPTL8 oligonucleotide including R, including wherein R comprises a sense or antisense strand, of an RNAi agent herein, or attached to one of the internal nucleotides. One of skill in the art will also recognize that the linker maybe linked or conjugated to the 5′ or 3′ end of an ANGPTL8 oligonucleotide including a sense or antisense strand herein. One of skill in the art will also recognize that placement of a delivery moiety, such as the delivery moieties comprising Formula I, whether via a linker or not, on the 5′ end an ANPTL8 oligonucleotide such as an antisense strand of an RNAi agent herein may need to overcome potential inefficient loading of Ago2 loading, or other hindrance of the RISC complex activity. For example, for a delivery moiety comprising Formula I linked or conjugated to the sense or antisense strand of an RNAi agent herein, such as an siRNA comprising a sense strand and an antisense strand, placement of the delivery moiety at the 5′ end of the antisense strand may create difficulties for Ago2 loading and prevent efficient knockdown. In a suitable embodiment, the one or more ANPTL8 oligonucleotides or RNAi agents comprise an siRNA comprising a sense strand and an antisense strand, and the delivery moiety comprising Formula I is present on the 3′ end of the sense strand. In a further embodiment, the delivery moiety comprising Formula I is conjugated to the 3′ end of the sense strand via a linker. In yet a further embodiment the linker comprises a ring structure, suitably a piperidine ring. In yet a further embodiment, the linker comprises Linker 1 (Formula II). In yet a further embodiment, the linker is Linker 2 (Formula III). In an embodiment Linker 1, connection point A, or Linker 2, connection point C, is conjugated to Formula I. In an embodiment Linker 1, connection point A, is conjugated to Formula I and connection point B is conjugated to R. In an embodiment Linker 2, connection point C, is conjugated to Formula I and connection point D is conjugated to R. In an embodiment Linker 1, connection point A, is conjugated to Formula I and connection point B is conjugated to a phosphate group which is conjugated to R. In an embodiment Linker 2, connection point C, is conjugated to Formula I and connection point D is conjugated to a phosphate group which is conjugated to R.

In certain embodiments, the ANPTL8 oligonucleotide such as an antisense strand is complementary to a sequence of Table 1, that is complementary to a sequence represented by any one of SEQ ID NO:3 to SEQ ID NO:123. In suitable embodiments, the ANPTL8 oligonucleotide is complementary to a sequence in Table 2, that is the sequence is complementary to a sequence represented by any one of SEQ ID NO:3, 6, 10, 11, 12, 13, 16, 18, 23, 24, 26, 27, 30, 31, 32, 33, 34, 35, 36, 37, 38, 39, 40, 41, 42, 43, 44, 46, 47, 49, 50, 52, 55, 56, 57, 61, 62, 63, 64, 65, 66, 67, 69, 70, 71, 72, 73, 74, 77, 88, 89, 90, 91, 93, 97, 98, 99, 100, 101, 102, 103, 104, 105, 106, or 107.

In further embodiments, the ANPTL8 oligonucleotide or RNAi agent herein comprises an siRNA comprising a sense strand and an antisense strand. In still further embodiments, the siRNA comprises a sense strand of Table 2, that is a sense strand comprising or having a sequence represented by any one of SEQ ID NO:124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, or 230. In other further embodiments the ANPTL8 oligonucleotide or RNAi agent herein comprises an antisense strand of Table 2, that is an antisense strand comprising or having a sequence represented by one of SEQ ID NO:231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, or 360. In still further embodiments, the siRNA comprises a sense strand and an antisense strand from one row of Table 11. In one of these further embodiments, the siRNA comprises a sense strand and an antisense strand from row 1, that is the siRNA comprises a sense strand comprising SEQ ID NO:124 and an antisense strand comprising SEQ ID NO:231. In other further embodiments, the siRNA comprises a sense strand and an antisense strand from row 2, that is the siRNA comprises a sense strand comprising SEQ ID NO:125 and an antisense strand comprising SEQ ID NO:232. In other further embodiments the siRNA comprises a sense strand and an antisense strand from the same row of Table 11, wherein the row is selected from the group consisting of rows 1 to 174 of Table 11 (rows labeled 1A to 174e5).

In further embodiments the sense strand and the antisense strand are modified. In further embodiments the modification is on the nucleotide, the backbone, i.e. the internucleoside linkage (phosphodiester bond), or both. In a further embodiment, the one or more modified internucleotide linkage is a phosphorothioate (PS) bond. In other embodiments, the internucleotide linkage is a modified internucleotide linkage that is a phosphorothioate (PS) bond. In further embodiments the modification is a 2′ fluoro group or a 2′ methoxy group on the ribose, or a PS bond, or both. In further embodiments, one or more of all three of these recited modifications are present. In further embodiments, the siRNA comprises between one to ten 2′ fluoro modifications on the ribose. In other embodiments the siRNA comprises between one to ten 2′ fluoro modifications on the ribose and the remainder of the nucleotides, that is, the nucleotides that do not have a 2′ fluoro modification, have a 2′ methoxy group modification on the ribose.

In other embodiments of the compounds such as the RNAi agents disclosed herein, the one or more oligonucleotides comprise an siRNA comprising a sense strand and an antisense strand. In a further embodiment, the sense strand and the antisense strand are each between 15-40 nucleotides in length. In suitable embodiments, the antisense strand is 23 nucleotides in length. In suitable embodiments, the sense strand is 21 nucleotides in length. In other suitable embodiments, the antisense strand is 23 nucleotides in length and the sense strand is 21 nucleotides in length. In another embodiment, the sense strand and the antisense strand anneal, and optionally comprise one or more 5′ or 3′ nucleotide overhangs, one or more 5′ or 3′ blunt ends, or a combination of both.

In another embodiment of the RNAi molecules including the RNAi agents disclosed herein, the 5′ or 3′ ends of the ANGPTL8 oligonucleotides, such as those represented by R, including wherein R comprises a sense strand and an antisense strand, are further modified. In a further embodiment, the 5′ end of the antisense strand is optionally phosphorylated. In a further embodiment, the nucleotide at 5′ end of the antisense strand comprises a 5′ vinylphosphonate modification. In another embodiment, the nucleotide at the 5′ end of the antisense strand comprises a phosphate group. In another embodiment, the nucleotide at the 5′ end of the antisense strand comprises a phosphate analog.

In other embodiments the RNAi molecules such as the RNAi agents disclosed herein comprise an siRNA that comprises Formula I and a sense or antisense strand of Table 6 or 8. In other embodiments, the RNAi molecule or RNAi agent, each disclosed herein, comprises Formula I and a sense strand comprising a sequence of any one of SEQ ID NO:361, SEQ ID NO:362, SEQ ID NO:363, SEQ ID NO:364, SEQ ID NO:365, or SEQ ID NO:366. In other embodiments, the RNAi molecule or RNAi agent, each disclosed herein, comprises Formula I and an siRNA comprising an antisense strand comprising a sequence of any one of SEQ ID NO:367, SEQ ID NO:368, SEQ ID NO:369, SEQ ID NO:370, SEQ ID NO:371, or SEQ ID NO:372. In a further embodiment, the RNAi molecule or RNAi agent, each disclosed herein, comprises Formula I and an siRNA comprising a sense strand and an antisense strand selected from the pairs of sequences as set forth in a-f:
a. SEQ ID NO:361 and SEQ ID NO:367;
b. SEQ ID NO:362 and SEQ ID NO:368;
c. SEQ ID NO:363 and SEQ ID NO:369;
d. SEQ ID NO:364 and SEQ ID NO:370;
e. SEQ ID NO:365 and SEQ ID NO:371; or
f. SEQ ID NO:366 and SEQ ID NO:372.

In still further embodiments, the RNAi molecules including the RNAi agent disclosed herein are conjugated to Formula I via a linker of Formula III (i.e. linker 2). In yet further embodiments, the linker of Formula III is conjugated to the nucleotide at the 3′ end of the sense strand. In further embodiments, any 5′ phosphate on the antisense strand is omitted, and one or more 2′ fluoro residues in the ribose are removed. In a further embodiment, any 2′ fluoro residues that are removed are replaced by 2′ methoxy modifications at the same position.

The RNAi molecules herein comprising Formula I and one or more ANGPTL8 oligonucleotides, such as those represented by R, including wherein R comprises a sense strand and an antisense strand, are useful in therapy, for diseases of the liver or involving adipose tissue. These are formulated into pharmaceutical compositions compatible for use in patients, suitably humans. The pharmaceutical compositions disclosed herein comprise one or more carriers, diluents, and excipients that are compatible with the RNAi molecules or RNAi agents herein and other components of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012.

Accordingly, in one embodiment is a pharmaceutical composition comprising an RNAi molecule or RNAi agent disclosed herein, comprising Formula I and one or more ANGPTL8 oligonucleotides, such as those represented by R, including wherein R comprises a sense strand and an antisense strand, and one or more pharmaceutically acceptable excipients. In further embodiments the RNA molecule such as an RNAi molecule or RNAi agent disclosed herein, and pharmaceutical compositions thereof, are for use in therapy or treatment of disease.

Another embodiment is the RNAi molecules or RNAi agents comprising Formula I and one or more ANGPTL8 oligonucleotides, such as those represented by R, including wherein R comprises a sense strand and an antisense strand, or pharmaceutical compositions thereof, for use in therapy. Another embodiment is an RNAi agent disclosed herein for use in therapy. In a further embodiment, the therapy involves decreasing levels of ANGPTL8 expression, such as relative to an untreated or control or placebo therapy. A further embodiment is wherein the therapy is for diseases of the liver or involving the liver. Another embodiment is a method of treatment of a liver disease comprising administering an RNAi molecule disclosed herein, suitably an RNAi molecule comprising Formula I and one or more ANGPTL8 oligonucleotides including R, such as wherein R comprises a sense strand and an antisense strand disclosed herein, suitably administered in an effective amount, or a pharmaceutical composition of any of the preceding. Another embodiment is a method of treating liver disease in a patient in need thereof comprising administering an RNAi agent disclosed herein, suitably an effective amount thereof, or a pharmaceutical composition comprising the RNAi agent and one or more pharmaceutically acceptable excipients.

Another embodiment is an RNA interference (RNAi) agent comprising Formula I:

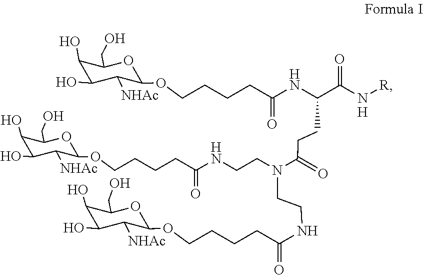

and a sense strand and an antisense strand, wherein the antisense strand comprises at least 18 contiguous nucleotides of a sequence selected from the group consisting of SEQ ID NOs: 405-525, and wherein the sense strand and the antisense strand form a region of complementarity of at least 15 nucleotides, and wherein the sense strand and antisense strand are each independently 18 to 23 nucleotides in length, and optionally wherein the sense strand and antisense strand each independently comprise one or more modified nucleotides, and optionally wherein one or more internucleotide linkages of the sense strand and the antisense strand are modified internucleotide linkages, and wherein the sense or the antisense strand is conjugated to Formula I, optionally via a linker.

The oligonucleotides such as the sense and antisense strands disclosed herein, may also be conjugated to alternative delivery moieties, for targeting to the liver, or to other tissues to decrease expression of ANGPTL8. The oligonucleotides such as the sense and antisense strands disclosed herein, may also be unconjugated, and instead encapsulated or delivered by another means not requiring conjugation, to a tissue of interest to decrease expression of ANGPTL8.

Accordingly, other embodiments of the RNAi agents disclosed herein RNAi agents comprising a delivery moiety conjugated to R, optionally via a linker L, having a formula R-L-D, wherein R comprises an antisense or a sense strand or both, and wherein the antisense strand comprises at least 15 contiguous nucleotides of a sequence that is complementary to the mRNA transcript of ANGPTL8, and wherein the sense strand and the antisense strand form a region of complementarity of at least 15 nucleotides, and wherein the sense strand and/or antisense strand, if and when present, are each independently 15 to 30 nucleotides in length, and optionally wherein the sense strand and antisense strand, if and when present, each independently comprise one or more modified nucleotides, and optionally wherein the sense strand and/or the antisense strand each independently comprise one or more modified internucleotide linkages. In another embodiment, the antisense strand comprises at least 15 contiguous nucleotides of a sequence that is complementary to SEQ ID NO:1. In a further embodiment, the sense strand and antisense strand are each independently 18 to 23 nucleotides in length. In a further embodiment of any of these RNAi agents, the anti sense strand forms a region of complementarity of at least 18 nucleotides to the mRNA transcript of ANGPTL8. In a different further embodiment of any of these RNAi agents, the antisense strand forms a region of complementarity of at least 18 nucleotides to SEQ ID NO:1.

In a further embodiment is an RNAi agent wherein the antisense strand comprises at least 15 nucleotides of a sequence selected from the group consisting of SEQ ID NO:s 405-525. In yet a further embodiment, the antisense strand comprises at least 18 contiguous nucleotides of a sequence selected from the group consisting of SEQ ID NOs: 405-525.

In a further embodiment of the RNAi agents disclosed herein, the antisense strand comprises at least 15 contiguous nucleotides of a sequence selected from the group consisting of SEQ ID NOs: 405, 408, 412, 413, 414, 415, 418, 420, 425, 426, 428, 429, 432, 433, 434, 435, 436, 437, 438, 439, 440, 441, 442, 443, 444, 445, 446, 448, 449, 451, 452, 454, 457, 458, 459, 463, 464, 465, 466, 467, 468, 469, 471, 472, 473, 474, 475, 476, 479, 490, 491, 492, 493, 495, 499, 500, 501,502, 503, 504, 505, 506, 507, 508, and 509. In another embodiment, the antisense strand is selected from the group of antisense strand sequences in Table 2.

In another embodiment of the RNAi agents disclosed herein, the antisense strand is selected from the group consisting of SEQ ID NOs: 231-361, or a sequence having at least 90% sequence identity thereto. In other embodiments of the RNAi agents disclosed herein, the sense strand is selected from the group consisting of SEQ ID NOs: 124-230, or a sequence having at least 90% sequence identity thereto.

In further embodiments of any of the RNAi agents described herein, the region of complementarity comprises 0, 1, 2, or 3 mismatches between the sense strand and the antisense strand.

In further embodiments of the RNAi agents, the sense strand and the antisense strand each independently comprise one or more modified nucleotides. In a further embodiment, the one or more modified nucleotides are independently 2′ fluoro modified nucleotides or 2′-O-methyl modified nucleotides. In other embodiments, each nucleotide of the sense strand and each nucleotide of the antisense strand is a modified nucleotide, and in further embodiment, each of the modified nucleotides are independently a 2′ fluoro modified nucleotide or a 2′-O-methyl modified nucleotide.

In other embodiments of the RNAi agents herein, the sense strand and antisense strand each independently comprise one or more modified internucleotide linkages. In a further embodiment, each modified internucleotide linkage is a phosphorothioate linkage. In other embodiments, the sense strand and antisense strand each independently comprise four phosphorothioate linkages. In further embodiments, the first two 5′ nucleotides of the sense strand and the two terminal 3′ nucleotides of the sense strand are phophorothioate linkages. In further embodiments, the first two 5′ nucleotides of the antisense strand and the two terminal 3′ nucleotides of the antisense strand are phosphorothioate linkages.

In other embodiments of the RNAi agents herein, the 5′ nucleotide of the antisense strand comprises a phosphate group or a phosphate analog.

A further embodiment of an RNAi agent disclosed herein is wherein the antisense strand comprises at least 18 contiguous nucleotides of a sequence selected from the group consisting of SEQ ID NOs: 405, 408, 412, 413, 414, 415, 418, 420, 425, 426, 428, 429, 432, 433, 434, 435, 436, 437, 438, 439, 440, 441, 442, 443, 444, 445, 446, 448, 449, 451, 452, 454, 457, 458, 459, 463, 464, 465, 466, 467, 468, 469, 471, 472, 473, 474, 475, 476, 479, 490, 491, 492, 493, 495, 499, 500, 501,502, 503, 504, 505, 506, 507, 508, and 509.

In another further embodiment, the RNAi agent comprises an antisense strand that is 23 nucleotides in length. In yet another further embodiment, the RNAi agent the sense strand is 21 nucleotides in length. In a suitable embodiment, the antisense strand is 23 nucleotides in length and the sense strand is 21 nucleotides in length.

In another embodiment, the antisense strand of the RNAi agent is selected from the group consisting of SEQ ID NOs: 231-361, or a sequence having at least 90% sequence identity thereto. In another embodiment, the sense strand of the RNAi agent is selected from the group consisting of SEQ ID NOs: 124-230, or a sequence having at least 90% sequence identity thereto. The RNAi agent of any one of the preceding claims, wherein, in the region of complementarity comprises 0, 1, 2, or 3 mismatches between the sense strand and the antisense strand.

In further embodiments, he RNAi agent comprises a sense strand or antisense strand comprising one or more modified nucleotides. In a further embodiment, the one or more modified nucleotides are independently a nucleotide having a 2′ fluoro group or having a 2′ O-methyl group on the ribose. In a further embodiment, each nucleotide of the sense strand and the antisense strand is a modified nucleotide.

In another embodiment of the RNAi agents herein, each of the two nucleotides at the 5′ and 3′ end of each of the sense strand and the antisense strand have a modified internucleotide linkage. In a further embodiment, the modified internucleotide linkage, if present, is a phosphorothiorate bond.

In another embodiment of the RNAi agents herein, the nucleotide at the 5′ end of the antisense strand has a modification or a further modification that is a phosphate group or a phosphate analog.

In further embodiments of the RNAi agents herein, the antisense strand comprises a sequence selected from the group consisting of SEQ ID NOs: 367-372, and 389-404, or a sequence having at least 90% sequence identity thereto.

In other further embodiments of the RNAi agents herein, the sense strand comprises a sequence selected from the group consisting of SEQ ID NOs: 361-366 and 373-388, or a sequence having at least 90% sequence identity thereto.

In other further embodiments of the RNAi agents disclosed herein, the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto;
b. a sense strand having the sequence set forth in SEQ ID NO:362, or a sequence having at least 90% sequence identity thereto, and the antisense strand having the sequence set forth in SEQ ID NO:368, or a sequence having at least 90% sequence identity thereto;
c. a sense strand having the sequence set forth in SEQ ID NO:363, or a sequence having at least 90% sequence identity thereto, and the antisense strand having the sequence set forth in SEQ ID NO:369, or a sequence having at least 90% sequence identity thereto;
d. a sense strand having the sequence set forth in SEQ ID NO:364, or a sequence having at least 90% sequence identity thereto, and the anti sense strand having the sequence set forth in SEQ ID NO:370, or a sequence having at least 90% sequence identity thereto;
e. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto;
f. a sense strand having the sequence set forth in SEQ ID NO:366, or a sequence having at least 90% sequence identity thereto, and the antisense strand having the sequence set forth in SEQ ID NO:372, or a sequence having at least 90% sequence identity thereto;
g. a sense strand having the sequence set forth in SEQ ID NO:373, or a sequence having at least 90% sequence identity thereto, and the antisense strand having the sequence set forth in SEQ ID NO:389, or a sequence having at least 90% sequence identity thereto;
h. a sense strand having the sequence set forth in SEQ ID NO:374, or a sequence having at least 90% sequence identity thereto, and an antisense strand having the sequence set forth in SEQ ID NO:, 390 or a sequence having at least 90% sequence identity thereto;
i. a sense strand having the sequence set forth in SEQ ID NO:375, or a sequence having at least 90% sequence identity thereto, and an antisense strand having the sequence set forth in SEQ ID NO:391, or a sequence having at least 90% sequence identity thereto;
j. a sense strand having the sequence set forth in SEQ ID NO:376, or a sequence having at least 90% sequence identity thereto, and an antisense strand having the sequence set forth in SEQ ID NO:392, or a sequence having at least 90% sequence identity thereto;
k. a sense strand having the sequence set forth in SEQ ID NO:377, or a sequence having at least 90% sequence identity thereto, and an antisense strand having the sequence set forth in SEQ ID NO:393, or a sequence having at least 90% sequence identity thereto;
l. a sense strand having the sequence set forth in SEQ ID NO:378, or a sequence having at least 90% sequence identity thereto, and an antisense strand having the sequence set forth in SEQ ID NO:394, or a sequence having at least 90% sequence identity thereto;
m. a sense strand having the sequence set forth in SEQ ID NO:379, or a sequence having at least 90% sequence identity thereto, and an antisense strand having the sequence set forth in SEQ ID NO:395, or a sequence having at least 90% sequence identity thereto;
n. a sense strand having the sequence set forth in SEQ ID NO:380 or a sequence having at least 90% sequence identity thereto, and an antisense strand having the sequence set forth in SEQ ID NO:396, or a sequence having at least 90% sequence identity thereto;
o. a sense strand having the sequence set forth in SEQ ID NO: 381, or a sequence having at least 90% sequence identity thereto, and an antisense strand having the sequence set forth in SEQ ID NO:396, or a sequence having at least 90% sequence identity thereto;
p. a sense strand having the sequence set forth in SEQ ID NO:382, or a sequence having at least 90% sequence identity thereto, and an antisense strand having the sequence set forth in SEQ ID NO:397, or a sequence having at least 90% sequence identity thereto;
q. a sense strand having the sequence set forth in SEQ ID NO:383, or a sequence having at least 90% sequence identity thereto, and an antisense strand having the sequence set forth in SEQ ID NO:398, or a sequence having at least 90% sequence identity thereto;
r. a sense strand having the sequence set forth in SEQ ID NO:384, or a sequence having at least 90% sequence identity thereto, and an antisense strand having the sequence set forth in SEQ ID NO:399, or a sequence having at least 90% sequence identity thereto;
s. a sense strand having the sequence set forth in SEQ ID NO:385, or a sequence having at least 90% sequence identity thereto, and an antisense strand having the sequence set forth in SEQ ID NO:400, or a sequence having at least 90% sequence identity thereto;
t. a sense strand having the sequence set forth in SEQ ID NO:386, or a sequence having at least 90% sequence identity thereto, and an antisense strand having the sequence set forth in SEQ ID NO:401, or a sequence having at least 90% sequence identity thereto;
u. a sense strand having the sequence set forth in SEQ ID NO:387, or a sequence having at least 90% sequence identity thereto, and an antisense strand having the sequence set forth in SEQ ID NO:402, or a sequence having at least 90% sequence identity thereto;
v. a sense strand having the sequence set forth in SEQ ID NO:388, or a sequence having at least 90% sequence identity thereto, and an antisense strand having the sequence set forth in SEQ ID NO:403, or a sequence having at least 90% sequence identity thereto; and
w. a sense strand having the sequence set forth in SEQ ID NO:389, or a sequence having at least 90% sequence identity thereto, and an antisense strand having the sequence set forth in SEQ ID NO:404, or a sequence having at least 90% sequence identity thereto;
   Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In other embodiments of the RNAi agents herein, the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto;
b. a sense strand having the sequence set forth in SEQ ID NO:362, or a sequence having at least 95% sequence identity thereto, and the antisense strand having the sequence set forth in SEQ ID NO:368, or a sequence having at least 95% sequence identity thereto;
c. a sense strand having the sequence set forth in SEQ ID NO:363, or a sequence having at least 95% sequence identity thereto, and the antisense strand having the sequence set forth in SEQ ID NO:369, or a sequence having at least 95% sequence identity thereto;
d. a sense strand having the sequence set forth in SEQ ID NO:364, or a sequence having at least 95% sequence identity thereto, and the antisense strand having the sequence set forth in SEQ ID NO:370, or a sequence having at least 95% sequence identity thereto;
e. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto;
f. a sense strand having the sequence set forth in SEQ ID NO:366, or a sequence having at least 95% sequence identity thereto, and the antisense strand having the sequence set forth in SEQ ID NO:372, or a sequence having at least 95% sequence identity thereto;
g. a sense strand having the sequence set forth in SEQ ID NO:373, or a sequence having at least 95% sequence identity thereto, and the antisense strand having the sequence set forth in SEQ ID NO:389, or a sequence having at least 95% sequence identity thereto;
h. a sense strand having the sequence set forth in SEQ ID NO:374, or a sequence having at least 95% sequence identity thereto, and an antisense strand having the sequence set forth in SEQ ID NO:, 390 or a sequence having at least 95% sequence identity thereto;
i. a sense strand having the sequence set forth in SEQ ID NO:375, or a sequence having at least 95% sequence identity thereto, and an antisense strand having the sequence set forth in SEQ ID NO:391, or a sequence having at least 95% sequence identity thereto;
j. a sense strand having the sequence set forth in SEQ ID NO:376, or a sequence having at least 95% sequence identity thereto, and an antisense strand having the sequence set forth in SEQ ID NO:392, or a sequence having at least 95% sequence identity thereto;
k. a sense strand having the sequence set forth in SEQ ID NO:377, or a sequence having at least 95% sequence identity thereto, and an antisense strand having the sequence set forth in SEQ ID NO:393, or a sequence having at least 95% sequence identity thereto;
l. a sense strand having the sequence set forth in SEQ ID NO:378, or a sequence having at least 95% sequence identity thereto, and an antisense strand having the sequence set forth in SEQ ID NO:394, or a sequence having at least 95% sequence identity thereto;
m. a sense strand having the sequence set forth in SEQ ID NO:379, or a sequence having at least 95% sequence identity thereto, and an antisense strand having the sequence set forth in SEQ ID NO:395, or a sequence having at least 95% sequence identity thereto;
n. a sense strand having the sequence set forth in SEQ ID NO:380, or a sequence having at least 95% sequence identity thereto, and an antisense strand having the sequence set forth in SEQ ID NO:396, or a sequence having at least 95% sequence identity thereto;
o. a sense strand having the sequence set forth in SEQ ID NO: 381, or a sequence having at least 95% sequence identity thereto, and an antisense strand having the sequence set forth in SEQ ID NO:396, or a sequence having at least 95% sequence identity thereto;
p. a sense strand having the sequence set forth in SEQ ID NO:382, or a sequence having at least 95% sequence identity thereto, and an antisense strand having the sequence set forth in SEQ ID NO:397, or a sequence having at least 95% sequence identity thereto;
q. a sense strand having the sequence set forth in SEQ ID NO:383, or a sequence having at least 95% sequence identity thereto, and an antisense strand having the sequence set forth in SEQ ID NO:398, or a sequence having at least 95% sequence identity thereto;
r. a sense strand having the sequence set forth in SEQ ID NO:384, or a sequence having at least 95% sequence identity thereto, and an antisense strand having the sequence set forth in SEQ ID NO:399, or a sequence having at least 95% sequence identity thereto;
s. a sense strand having the sequence set forth in SEQ ID NO:385, or a sequence having at least 95% sequence identity thereto, and an antisense strand having the sequence set forth in SEQ ID NO:400, or a sequence having at least 95% sequence identity thereto;
t. a sense strand having the sequence set forth in SEQ ID NO:386, or a sequence having at least 95% sequence identity thereto, and an antisense strand having the sequence set forth in SEQ ID NO:401, or a sequence having at least 95% sequence identity thereto;
u. a sense strand having the sequence set forth in SEQ ID NO:387, or a sequence having at least 95% sequence identity thereto, and an antisense strand having the sequence set forth in SEQ ID NO:402, or a sequence having at least 95% sequence identity thereto;
v. a sense strand having the sequence set forth in SEQ ID NO:388, or a sequence having at least 95% sequence identity thereto, and an antisense strand having the sequence set forth in SEQ ID NO:403, or a sequence having at least 95% sequence identity thereto; and
w. a sense strand having the sequence set forth in SEQ ID NO:389, or a sequence having at least 95% sequence identity thereto, and an antisense strand having the sequence set forth in SEQ ID NO:404, or a sequence having at least 95% sequence identity thereto:
   Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In other embodiments of the RNAi agents disclosed herein, the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto;
b. a sense strand having the sequence set forth in SEQ ID NO:362, or a sequence having at least 90% sequence identity thereto, and the antisense strand having the sequence set forth in SEQ ID NO:368, or a sequence having at least 90% sequence identity thereto;
c. a sense strand having the sequence set forth in SEQ ID NO:363, or a sequence having at least 90% sequence identity thereto, and the antisense strand having the sequence set forth in SEQ ID NO:369, or a sequence having at least 90% sequence identity thereto;
d. a sense strand having the sequence set forth in SEQ ID NO:364, or a sequence having at least 90% sequence identity thereto, and the antisense strand having the sequence set forth in SEQ ID NO:370, or a sequence having at least 90% sequence identity thereto; and
e. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto;

Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In a further embodiment of the RNAi agents disclosed herein, the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto;
b. a sense strand having the sequence set forth in SEQ ID NO:362, or a sequence having at least 95% sequence identity thereto, and the antisense strand having the sequence set forth in SEQ ID NO:368, or a sequence having at least 95% sequence identity thereto;
c. a sense strand having the sequence set forth in SEQ ID NO:363, or a sequence having at least 95% sequence identity thereto, and the antisense strand having the sequence set forth in SEQ ID NO:369, or a sequence having at least 95% sequence identity thereto;
d. a sense strand having the sequence set forth in SEQ ID NO:364, or a sequence having at least 95% sequence identity thereto, and the antisense strand having the sequence set forth in SEQ ID NO:370, or a sequence having at least 95% sequence identity thereto; and
e. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto.
   Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In another embodiment of the RNAi agents disclosed herein, is an RNA agent wherein the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto; and
b. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto.
   Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In other embodiments of the RNAi agents disclosed herein, the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto; and
b. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto.
   Suitably, the RNAi agents having the pair of recited oligonucleotide sequences comprise Formula I. In further suitable embodiments, the RNAi agents comprise Formula I conjugated to the nucleotide at the 3′ end of the sense strand. In yet further embodiments, Formula I is conjugated to the nucleotide at the 3′ end of the sense strand via a linker. In further embodiments, the linker is a linker of Formula III.

In other embodiments herein, the RNAi agent is capable of decreasing expression of the ANGPTL8 gene in a liver cell.

In other embodiments, the RNAi agent disclosed herein is provided for use in therapy. Another embodiment is an RNAi molecule or RNAi agent disclosed herein, suitably comprising Formula I and one or more ANGPTL8 oligonucleotides including R, such as wherein R comprises a sense strand and/or an antisense strand disclosed herein, or a pharmaceutical composition thereof, for use in the manufacture of a medicament, suitably for the treatment of liver disease or a disease involving the liver, each including dyslipidemia, cardiovascular disease, and non-alcoholic fatty liver disease (NAFLD). In another embodiment is the use of an RNAi molecule or RNAi agent disclosed herein, suitably comprising Formula I and one or more ANGPTL8 oligonucleotides, including R, such as wherein R comprises a sense strand and/or an antisense strand disclosed herein, or a pharmaceutical composition thereof, in the manufacture of a medicament, suitably for the treatment of liver disease or a disease involving the liver, each including dyslipidemia, cardiovascular disease, and non-alcoholic fatty liver disease (NAFLD). In a further embodiment, the NAFLD is non-alcoholic steatohepatitis (NASH). Another embodiment is an RNAi agent disclosed herein, or a pharmaceutical composition thereof, for use in the manufacture of a medicament, suitably for the treatment of liver disease or a disease involving the liver, each including dyslipidemia, cardiovascular disease, and non-alcoholic fatty liver disease (NAFLD). In a further embodiment, the NAFLD is non-alcoholic steatohepatitis (NASH).

In other embodiments are methods of treating dyslipidemia comprising administering an effective amount of an RNAi molecule disclosed herein, or a pharmaceutical composition thereof, to a patient in need thereof. Another embodiment is an RNAi molecule disclosed herein, or a pharmaceutical composition thereof, for use in treating dyslipidemia. In other embodiments are methods of treating dyslipidemia comprising administering an effective amount of an RNAi agent disclosed herein, or a pharmaceutical composition thereof, to a patient in need thereof. Another embodiment is an RNAi agent disclosed herein, or a pharmaceutical composition thereof, for use in treating dyslipidemia.

In other embodiments are provided methods of treating cardiovascular disease comprising administering an effective amount of the RNAi molecule disclosed herein, or a pharmaceutical composition thereof, to a patient in need thereof. Another embodiment is an RNAi molecule disclosed herein, or a pharmaceutical composition thereof, for use in treating cardiovascular disease, optionally and suitably as measured by a decrease in hospitalizations and/or cardiovascular events and/or risk of either or both. In other embodiments are provided methods of treating cardiovascular disease comprising administering an effective amount of an RNAi agent disclosed herein, or a pharmaceutical composition thereof, to a patient in need thereof. Another embodiment is an RNAi agent disclosed herein, or a pharmaceutical composition thereof, for use in treating cardiovascular disease, optionally and suitably as measured by a decrease in hospitalizations and/or cardiovascular events and/or risk of either or both.

In other embodiments are methods preventing a cardiovascular event, comprising administering an effective amount of an RNAi molecule disclosed herein or a pharmaceutical composition thereof, to a patient in need thereof. In other embodiments are methods preventing a cardiovascular event, comprising administering an effective amount of an RNAi agent disclosed herein or a pharmaceutical composition thereof, to a patient in need thereof. In further embodiments the cardiovascular event is myocardial infarction.

In other embodiments are methods of decreasing hospitalizations related to cardiovascular disease or events comprising administering an effective amount of an RNAi molecule disclosed herein, such as an RNAi agent disclosed herein, or a pharmaceutical composition thereof, to a patient in need thereof.

In other embodiments are methods of treating non-alcoholic fatty liver disease (NAFLD) comprising administering an effective amount of an RNAi molecule such as an RNAi agent disclosed herein, or a pharmaceutical composition thereof, to a patient in need thereof. Another embodiment is an RNAi molecule such as an RNAi agent disclosed herein, or a pharmaceutical composition thereof, for use in treating NAFLD. In further embodiments, the NAFLD is NASH.

In other embodiments are methods of lowering triglyceride levels, comprising administering an effective amount of an RNAi molecule such as an RNAi agent disclosed herein, or a pharmaceutical composition thereof, to a patient in need thereof.

In other embodiments are methods of inhibiting lipoprotein lipase (LPL) comprising administering an effective amount of an RNAi molecule such as an RNAi agent disclosed herein, or a pharmaceutical composition thereof, to a patient in need thereof.

In other embodiments are methods of increasing catabolism of triglyceride rich lipoproteins comprising administering an effective amount of an RNAi molecule such as an RNAi agent disclosed herein, or a pharmaceutical composition thereof, to a patient in need thereof.

In other embodiments are methods of treating liver disease in a patient that would benefit from decreasing expression levels of ANGPTL8, comprising administering an effective amount of an RNAi molecule such as an RNAi agent disclosed herein or a pharmaceutical composition thereof, to a patient in need thereof.

As used herein, the term “effective amount” refers to an amount that is effective in treating a disorder or disease.

As used herein, the term “treat” or “treating” means an act of providing care to an individual in need thereof, for example, by administering a therapeutic agent (e.g., an oligonucleotide herein) to the individual for purposes of improving the health and/or well-being of the individual with respect to an existing condition (e.g., a disease, disorder) or to prevent or decrease the likelihood of the occurrence of a condition. Treating also can involve reducing the frequency or severity of at least one sign, symptom or contributing factor of a condition (e.g., disease, disorder) experienced by the individual.

The RNAi agents are further described in the nonlimiting examples herein.

### EXAMPLE 1

TABLE 1
Target and antisense sequences for
designed siRNAs

| SEQ | Start | End | | SEQ | 18 mer |
|---|---|---|---|---|---|
| ID | Posi- | Posi | 18mer Target | ID | Antisense |
| NO | tion | tion | DNA | NO | RNA |
| (DNA | on | on | Sequence | (RNA | sequence |
| SEQ) | DNA | DNA | (5′-3′) | SEQ) | (5′-3′) |
| 3 | 120 | 137 | AGCTGACCCTGCTCTTCC | 405 | GGAAGAGCAG |
| | | | | | GGUCAGCU |
| 4 | 141 | 158 | GGACCCTGCAGCTGGGCC | 406 | GGCCCAGCUG |
| | | | | | CAGGGUCC |
| 5 | 150 | 167 | AGCTGGGCCAGGCCCTCA | 407 | UGAGGGCCUG |
| | | | | | GCCCAGCU |
| 6 | 185 | 202 | ACGGAGGGACGGCTGACA | 408 | UGUCAGCCGU |
| | | | | | CCCUCCGU |
| 7 | 186 | 203 | CGGAGGGACGGCTGACAA | 409 | UUGUCAGCCG |
| | | | | | UCCCUCCG |
| 8 | 191 | 208 | GGACGGCTGACAAAGGCC | 410 | GGCCUUUGUC |
| | | | | | AGCCGUCC |
| 9 | 194 | 211 | CGGCTGACAAAGGCCAGG | 411 | CCUGGCCUUU |
| | | | | | GUCAGCCG |
| 10 | 195 | 212 | GGCTGACAAAGGCCAGGA | 412 | UCCUGGCCUU |
| | | | | | UGUCAGCC |
| 11 | 197 | 214 | CTGACAAAGGCCAGGAAC | 413 | GUUCCUGGCC |
| | | | | | UUUGUCAG |
| 12 | 210 | 227 | GGAACAGCCTGGGTCTCT | 414 | AGAGACCCAG |
| | | | | | GCUGUUCC |
| 13 | 211 | 228 | GAACAGCCTGGGTCTCTA | 415 | UAGAGACCCA |
| | | | | | GGCUGUUC |
| 14 | 215 | 232 | AGCCTGGGTCTCTATGGC | 416 | GCCAUAGAGA |
| | | | | | CCCAGGCU |
| 15 | 217 | 234 | CCTGGGTCTCTATGGCCG | 417 | CGGCCAUAGA |
| | | | | | GACCCAGG |
| 16 | 218 | 235 | CTGGGTCTCTATGGCCGC | 418 | GCGGCCAUAG |
| | | | | | AGACCCAG |
| 17 | 219 | 236 | TGGGTCTCTATGGCCGCA | 419 | UGCGGCCAUA |
| | | | | | GAGACCCA |
| 18 | 220 | 237 | GGGTCTCTATGGCCGCAC | 420 | GUGCGGCCAU |
| | | | | | AGAGACCC |
| 19 | 259 | 276 | GGTCAGCCGGGGCCGGGA | 421 | UCCCGGCCCC |
| | | | | | GGCUGACC |
| 20 | 267 | 284 | GGGGCCGGGATGCAGCCC | 422 | GGGCUGCAUC |
| | | | | | CCGGCCCC |
| 21 | 270 | 287 | GCCGGGATGCAGCCCAGG | 423 | CCUGGGCUGC |
| | | | | | AUCCCGGC |
| 22 | 271 | 288 | CCGGGATGCAGCCCAGGA | 424 | UCCUGGGCUG |
| | | | | | CAUCCCGG |
| 23 | 273 | 290 | GGGATGCAGCCCAGGAAC | 425 | GUUCCUGGGC |
| | | | | | UGCAUCCC |
| 24 | 274 | 291 | GGATGCAGCCCAGGAACT | 426 | AGUUCCUGGG |
| | | | | | CUGCAUCC |
| 25 | 281 | 298 | GCCCAGGAACTTCGGGCA | 427 | UGCCCGAAGU |
| | | | | | UCCUGGGC |
| 26 | 282 | 299 | CCCAGGAACTTCGGGCAA | 428 | UUGCCCGAAG |
| | | | | | UUCCUGGG |
| 27 | 283 | 300 | CCAGGAACTTCGGGCAAG | 429 | CUUGCCCGAA |
| | | | | | GUUCCUGG |
| 28 | 284 | 301 | CAGGAACTTCGGGCAAGC | 430 | GCUUGCCCGA |
| | | | | | AGUUCCUG |
| 29 | 285 | 302 | AGGAACTTCGGGCAAGCC | 431 | GGCUUGCCCG |
| | | | | | AAGUUCCU |
| 30 | 286 | 303 | GGAACTTCGGGCAAGCCT | 432 | AGGCUUGCCC |
| | | | | | GAAGUUCC |
| 31 | 287 | 304 | GAACTTCGGGCAAGCCTG | 433 | CAGGCUUGCC |
| | | | | | CGAAGUUC |
| 32 | 290 | 307 | CTTCGGGCAAGCCTGTTG | 434 | CAACAGGCUU |
| | | | | | GCCCGAAG |
| 33 | 291 | 308 | TTCGGGCAAGCCTGTTGG | 435 | CCAACAGGCU |
| | | | | | UGCCCGAA |
| 34 | 293 | 310 | CGGGCAAGCCTGTTGGAG | 436 | CUCCAACAGG |
| | | | | | CUUGCCCG |
| 35 | 297 | 314 | CAAGCCTGTTGGAGACTC | 437 | GAGUCUCCAA |
| | | | | | CAGGCUUG |
| 36 | 299 | 316 | AGCCTGTTGGAGACTCAG | 438 | CUGAGUCUCC |
| | | | | | AACAGGCU |
| 37 | 300 | 317 | GCCTGTTGGAGACTCAGA | 439 | UCUGAGUCUC |
| | | | | | CAACAGGC |
| 38 | 303 | 320 | TGTTGGAGACTCAGATGG | 440 | CCAUCUGAGU |
| | | | | | CUCCAACA |
| 39 | 304 | 321 | GTTGGAGACTCAGATGGA | 441 | UCCAUCUGAG |
| | | | | | UCUCCAAC |
| 40 | 309 | 326 | AGACTCAGATGGAGGAGG | 442 | CCUCCUCCAU |
| | | | | | CUGAGUCU |
| 41 | 310 | 327 | GACTCAGATGGAGGAGGA | 443 | UCCUCCUCCA |
| | | | | | UCUGAGUC |
| 42 | 311 | 328 | ACTCAGATGGAGGAGGAT | 444 | AUCCUCCUCC |
| | | | | | AUCUGAGU |
| 43 | 312 | 329 | CTCAGATGGAGGAGGATA | 445 | UAUCCUCCUC |
| | | | | | CAUCUGAG |
| 44 | 313 | 330 | TCAGATGGAGGAGGATAT | 446 | AUAUCCUCCU |
| | | | | | CCAUCUGA |
| 45 | 314 | 331 | CAGATGGAGGAGGATATT | 447 | AAUAUCCUCC |
| | | | | | UCCAUCUG |
| 46 | 315 | 332 | AGATGGAGGAGGATATTC | 448 | GAAUAUCCUC |
| | | | | | CUCCAUCU |
| 47 | 316 | 333 | GATGGAGGAGGATATTCT | 449 | AGAAUAUCCU |
| | | | | | CCUCCAUC |
| 48 | 317 | 334 | ATGGAGGAGGATATTCTG | 450 | CAGAAUAUCC |
| | | | | | UCCUCCAU |
| 49 | 319 | 336 | GGAGGAGGATATTCTGCA | 451 | UGCAGAAUAU |
| | | | | | CCUCCUCC |
| 50 | 321 | 338 | AGGAGGATATTCTGCAGC | 452 | GCUGCAGAAU |
| | | | | | AUCCUCCU |
| 51 | 372 | 389 | AGGTGGCCCAGGCACAGA | 453 | UCUGUGCCUG |
| | | | | | GGCCACCU |
| 52 | 379 | 396 | CCAGGCACAGAAGGTGCT | 454 | AGCACCUUCU |
| | | | | | GUGCCUGG |
| 53 | 411 | 428 | AGCGGCTAGAAGTCCAGC | 455 | GCUGGACUUC |
| | | | | | UAGCCGCU |
| 54 | 412 | 429 | GCGGCTAGAAGTCCAGCT | 456 | AGCUGGACUU |
| | | | | | CUAGCCGC |
| 55 | 413 | 430 | CGGCTAGAAGTCCAGCTG | 457 | CAGCUGGACU |
| | | | | | UCUAGCCG |
| 56 | 414 | 431 | GGCTAGAAGTCCAGCTGA | 458 | UCAGCUGGAC |
| | | | | | UUCUAGCC |
| 57 | 415 | 432 | GCTAGAAGTCCAGCTGAG | 459 | CUCAGCUGGA |
| | | | | | CUUCUAGC |
| 58 | 420 | 437 | AAGTCCAGCTGAGGAGCG | 460 | CGCUCCUCAG |
| | | | | | CUGGACUU |
| 59 | 421 | 438 | AGTCCAGCTGAGGAGCGC | 461 | GCGCUCCUCA |
| | | | | | GCUGGACU |
| 60 | 438 | 455 | CCTGGCTGGGCCCTGCCT | 462 | AGGCAGGGCC |
| | | | | | CAGCCAGG |
| 61 | 459 | 476 | GAGAATTTGAGGTCTTAA | 463 | UUAAGACCUC |
| | | | | | AAAUUCUC |
| 62 | 460 | 477 | AGAATTTGAGGTCTTAAA | 464 | UUUAAGACCU |
| | | | | | CAAAUUCU |
| 63 | 462 | 479 | AATTTGAGGTCTTAAAGG | 465 | CCUUUAAGAC |
| | | | | | CUCAAAUU |
| 64 | 463 | 480 | ATTTGAGGTCTTAAAGGC | 466 | GCCUUUAAGA |
| | | | | | CCUCAAAU |
| 65 | 464 | 481 | TTTGAGGTCTTAAAGGCT | 467 | AGCCUUUAAG |
| | | | | | ACCUCAAA |
| 66 | 465 | 482 | TTGAGGTCTTAAAGGCTC | 468 | GAGCCUUUAA |
| | | | | | GACCUCAA |
| 67 | 466 | 483 | TGAGGTCTTAAAGGCTCA | 469 | UGAGCCUUUA |
| | | | | | AGACCUCA |
| 68 | 467 | 484 | GAGGTCTTAAAGGCTCAC | 470 | GUGAGCCUUU |
| | | | | | AAGACCUC |
| 69 | 469 | 486 | GGTCTTAAAGGCTCACGC | 471 | GCGUGAGCCU |
| | | | | | UUAAGACC |
| 70 | 471 | 488 | TCTTAAAGGCTCACGCTG | 472 | CAGCGUGAGC |
| | | | | | CUUUAAGA |
| 71 | 472 | 489 | CTTAAAGGCTCACGCTGA | 473 | UCAGCGUGAG |
| | | | | | CCUUUAAG |
| 72 | 473 | 490 | TTAAAGGCTCACGCTGAC | 474 | GUCAGCGUGA |
| | | | | | GCCUUUAA |
| 73 | 474 | 491 | TAAAGGCTCACGCTGACA | 475 | UGUCAGCGUG |
| | | | | | AGCCUUUA |
| 74 | 475 | 492 | AAAGGCTCACGCTGACAA | 476 | UUGUCAGCGU |
| | | | | | GAGCCUUU |
| 75 | 476 | 493 | AAGGCTCACGCTGACAAG | 477 | CUUGUCAGCG |
| | | | | | UGAGCCUU |
| 76 | 477 | 494 | AGGCTCACGCTGACAAGC | 478 | GCUUGUCAGC |
| | | | | | GUGAGCCU |
| 77 | 479 | 496 | GCTCACGCTGACAAGCAG | 479 | CUGCUUGUCA |
| | | | | | GCGUGAGC |
| 78 | 480 | 497 | CTCACGCTGACAAGCAGA | 480 | UCUGCUUGUC |
| | | | | | AGCGUGAG |
| 79 | 481 | 498 | TCACGCTGACAAGCAGAG | 481 | CUCUGCUUGU |
| | | | | | CAGCGUGA |
| 80 | 482 | 499 | CACGCTGACAAGCAGAGC | 482 | GCUCUGCUUG |
| | | | | | UCAGCGUG |
| 81 | 483 | 500 | ACGCTGACAAGCAGAGCC | 483 | GGCUCUGCUU |
| | | | | | GUCAGCGU |
| 82 | 485 | 502 | GCTGACAAGCAGAGCCAC | 484 | GUGGCUCUGC |
| | | | | | UUGUCAGC |
| 83 | 513 | 530 | CCCTCACAGGCCACGTGC | 485 | GCACGUGGCC |
| | | | | | UGUGAGGG |
| 84 | 514 | 531 | CCTCACAGGCCACGTGCA | 486 | UGCACGUGGC |
| | | | | | CUGUGAGG |
| 85 | 520 | 537 | AGGCCACGTGCAGCGGCA | 487 | UGCCGCUGCA |
| | | | | | CGUGGCCU |
| 86 | 521 | 538 | GGCCACGTGCAGCGGCAG | 488 | CUGCCGCUGC |
| | | | | | ACGUGGCC |
| 87 | 524 | 541 | CACGTGCAGCGGCAGAGG | 489 | CCUCUGCCGC |
| | | | | | UGCACGUG |
| 88 | 530 | 547 | CAGCGGCAGAGGCGGGAG | 490 | CUCCCGCCUC |
| | | | | | UGCCGCUG |
| 89 | 534 | 551 | GGCAGAGGCGGGAGATGG | 491 | CCAUCUCCCG |
| | | | | | CCUCUGCC |
| 90 | 540 | 557 | GGCGGGAGATGGTGGCAC | 492 | GUGCCACCAU |
| | | | | | CUCCCGCC |
| 91 | 543 | 560 | GGGAGATGGTGGCACAGC | 493 | GCUGUGCCAC |
| | | | | | CAUCUCCC |
| 92 | 546 | 563 | AGATGGTGGCACAGCAGC | 494 | GCUGCUGUGC |
| | | | | | CACCAUCU |
| 93 | 553 | 570 | GGCACAGCAGCATCGGCT | 495 | AGCCGAUGCU |
| | | | | | GCUGUGCC |
| 94 | 555 | 572 | CACAGCAGCATCGGCTGC | 496 | GCAGCCGAUG |
| | | | | | CUGCUGUG |
| 95 | 557 | 574 | CAGCAGCATCGGCTGCGA | 497 | UCGCAGCCGA |
| | | | | | UGCUGCUG |
| 96 | 558 | 575 | AGCAGCATCGGCTGCGAC | 498 | GUCGCAGCCG |
| | | | | | AUGCUGCU |
| 97 | 559 | 576 | GCAGCATCGGCTGCGACA | 499 | UGUCGCAGCC |
| | | | | | GAUGCUGC |
| 98 | 560 | 577 | CAGCATCGGCTGCGACAG | 500 | CUGUCGCAGC |
| | | | | | CGAUGCUG |
| 99 | 561 | 578 | AGCATCGGCTGCGACAGA | 501 | UCUGUCGCAG |
| | | | | | CCGAUGCU |
| 100 | 562 | 579 | GCATCGGCTGCGACAGAT | 502 | AUCUGUCGCA |
| | | | | | GCCGAUGC |
| 101 | 563 | 580 | CATCGGCTGCGACAGATC | 503 | GAUCUGUCGC |
| | | | | | AGCCGAUG |
| 102 | 564 | 581 | ATCGGCTGCGACAGATCC | 504 | GGAUCUGUCG |
| | | | | | CAGCCGAU |
| 103 | 565 | 582 | TCGGCTGCGACAGATCCA | 505 | UGGAUCUGUC |
| | | | | | GCAGCCGA |
| 104 | 567 | 584 | GGCTGCGACAGATCCAGG | 506 | CCUGGAUCUG |
| | | | | | UCGCAGCC |
| 105 | 569 | 586 | CTGCGACAGATCCAGGAG | 507 | CUCCUGGAUC |
| | | | | | UGUCGCAG |
| 106 | 570 | 587 | TGCGACAGATCCAGGAGA | 508 | UCUCCUGGAU |
| | | | | | CUGUCGCA |
| 107 | 571 | 588 | GCGACAGATCCAGGAGAG | 509 | CUCUCCUGGA |
| | | | | | UCUGUCGC |
| 108 | 607 | 624 | CCCAGCCTGAATCTGCCT | 510 | AGGCAGAUUC |
| | | | | | AGGCUGGG |
| 109 | 610 | 627 | AGCCTGAATCTGCCTGGA | 511 | UCCAGGCAGA |
| | | | | | UUCAGGCU |
| 110 | 611 | 628 | GCCTGAATCTGCCTGGAT | 512 | AUCCAGGCAG |
| | | | | | AUUCAGGC |
| 111 | 619 | 636 | CTGCCTGGATGGAACTGA | 513 | UCAGUUCCAU |
| | | | | | CCAGGCAG |
| 112 | 644 | 661 | TCATGOTGCAAGGAACAC | 514 | GUGUUCCUUG |
| | | | | | CAGCAUGA |
| 113 | 652 | 669 | CAAGGAACACTTCCACGC | 515 | GCGUGGAAGU |
| | | | | | GUUCCUUG |
| 114 | 655 | 672 | GGAACACTTCCACGCCCC | 516 | GGGGCGUGGA |
| | | | | | AGUGUUCC |
| 115 | 698 | 715 | TGCCTGTTCACTGGGATC | 517 | GAUCCCAGUG |
| | | | | | AACAGGCA |
| 116 | 699 | 716 | GCCTGTTCACTGGGATCA | 518 | UGAUCCCAGU |
| | | | | | GAACAGGC |
| 117 | 701 | 718 | CTGTTCACTGGGATCAGC | 519 | GCUGAUCCCA |
| | | | | | GUGAACAG |
| 118 | 702 | 719 | TGTTCACTGGGATCAGCC | 520 | GGCUGAUCCC |
| | | | | | AGUGAACA |
| 119 | 709 | 726 | TGGGATCAGCCAGGGCGC | 521 | GCGCCCUGGC |
| | | | | | UGAUCCCA |
| 120 | 710 | 727 | GGGATCAGCCAGGGCGCC | 522 | GGCGCCCUGG |
| | | | | | CUGAUCCC |
| 121 | 847 | 864 | CATTAAAGCAGAGTCGTG | 523 | CACGACUCUG |
| | | | | | CUUUAAUG |
| 122 | 848 | 865 | ATTAAAGCAGAGTCGTGG | 524 | CCACGACUCU |
| | | | | | GCUUUAAU |
| 123 | 850 | 867 | TAAAGCAGAGTCGTGGCA | 525 | UGCCACGACU |
| | | | | | CUGCUUUA |

siRNAs are designed that are complementary to the 18mer regions of the ANGPTL8 transcript NM (SEQ ID NO: 1) as shown above in Table 1. The sense strand and the antisense RNA oligonucleotides strands are between 18 and 23 nucleotides in length, with optional overhangs of 1 to 5 ribonucleotides, with 1-10 fluoro additions at the 2′ position of ribose, and the remaining residues are methylated at the 2′position of ribose (creating an 2′ methoxy i.e. a 2′ O-methyl modification). Some antisense strands are phosphorylated at the 5′ position. Each siRNA is conjugated to a delivery moiety comprising 3 GalNAc group; select delivery moieties comprise Formula I while others comprise a control moiety. One or more phosphodiester bonds are present at the 5′ and 3′ ends.

Knockdown of ANGPTL8 expression by these siRNAs is assayed using the following procedure: mouse primary hepatocytes (MPH) are freshly isolated from an AAV-ANGPTL8 humanized mouse, added to Corning plates at 15k per well, and siRNA are added directly to the well. For single measurements, 1 uM (1000 nM) is used; to generate concentration/dose response curves final concentrations of 1000, 333, 111, 37, 12, 4, 1.37, 0.46, 0.15, 0.05, and 0.017 nM of GalNAc-conjugated siRNA concentration is used.

Treated cells are lysed and RNA is isolated using the Quick-RNA 96 Kit (Zymo Research) directly into the 96 well plate. The eluted RNA is used immediately or stored frozen. cDNA is synthesized using Fast Advanced RT Master Mix (Invitrogen) and using the following steps in a thermocycler: 37 C for 30 minutes, 95 C for 5 minutes, and 4 C hold. Polymerase Chain Reaction (PCR) is performed via TaqMan RT PCR (Life Technologies) using the following cycles temperatures and times: 50 C for 2 minutes, 95 C for 10 minutes, 40 cycles of 95 C for 15 seconds and 60 C for 1 minute.

The human ANGPTL8 levels are normalized to mouse Rp1p0 (Life Technologies) and represent the relative knockdown of human ANGPTL8 mRNA expression as compared to vehicle-treated control cells. IC50 values are calculated using a 4-parameter fit model using XLFit.

The ANGPTL8 target regions of siRNAs that exhibited a greater than 50% knockdown are shown below in Table 2.

TABLE 2

| SEQ | | | | SEQ | |
|---|---|---|---|---|---|
| ID NO | Start | End | 18 mer DNA | ID NO | Antisense RNA |
| (DNA | Position | Position | Sequence | (RNA | sequence |
| SEQ) | on DNA | on DNA | (5′-3′) | SEQ) | (5′-3′) |
| 3 | 120 | 137 | AGCTGACCCTGCTCTTCC | 405 | GGAAGAGCAGGGUCAGCU |
| 6 | 185 | 202 | ACGGAGGGACGGCTGACA | 408 | UGUCAGCCGUCCCUCCGU |
| 10 | 195 | 212 | GGCTGACAAAGGCCAGGA | 412 | UCCUGGCCUUUGUCAGCC |
| 11 | 197 | 214 | CTGACAAAGGCCAGGAAC | 413 | GUUCCUGGCCUUUGUCAG |
| 12 | 210 | 227 | GGAACAGCCTGGGTCTCT | 414 | AGAGACCCAGGCUGUUCC |
| 13 | 211 | 228 | GAACAGCCTGGGTCTCTA | 415 | UAGAGACCCAGGCUGUUC |
| 16 | 218 | 235 | CTGGGTCTCTATGGCCGC | 418 | GCGGCCAUAGAGACCCAG |
| 18 | 220 | 237 | GGGTCTCTATGGCCGCAC | 420 | GUGCGGCCAUAGAGACCC |
| 23 | 273 | 290 | GGGATGCAGCCCAGGAAC | 425 | GUUCCUGGGCUGCAUCCC |
| 24 | 274 | 291 | GGATGCAGCCCAGGAACT | 426 | AGUUCCUGGGCUGCAUCC |
| 26 | 282 | 299 | CCCAGGAACTTCGGGCAA | 428 | UUGCCCGAAGUUCCUGGG |
| 27 | 283 | 300 | CCAGGAACTTCGGGCAAG | 429 | CUUGCCCGAAGUUCCUGG |
| 30 | 286 | 303 | GGAACTTCGGGCAAGCCT | 432 | AGGCUUGCCCGAAGUUCC |
| 31 | 287 | 304 | GAACTTCGGGCAAGCCTG | 433 | CAGGCUUGCCCGAAGUUC |
| 32 | 290 | 307 | CTTCGGGCAAGCCTGTTG | 434 | CAACAGGCUUGCCCGAAG |
| 33 | 291 | 308 | TTCGGGCAAGCCTGTTGG | 435 | CCAACAGGCUUGCCCGAA |
| 34 | 293 | 310 | CGGGCAAGCCTGTTGGAG | 436 | CUCCAACAGGCUUGCCCG |
| 35 | 297 | 314 | CAAGCCTGTTGGAGACTC | 437 | GAGUCUCCAACAGGCUUG |
| 36 | 299 | 316 | AGCCTGTTGGAGACTCAG | 438 | CUGAGUCUCCAACAGGCU |
| 37 | 300 | 317 | GCCTGTTGGAGACTCAGA | 439 | UCUGAGUCUCCAACAGGC |
| 38 | 303 | 320 | TGTTGGAGACTCAGATGG | 440 | CCAUCUGAGUCUCCAACA |
| 39 | 304 | 321 | GTTGGAGACTCAGATGGA | 441 | UCCAUCUGAGUCUCCAAC |
| 40 | 309 | 326 | AGACTCAGATGGAGGAGG | 442 | CCUCCUCCAUCUGAGUCU |
| 41 | 310 | 327 | GACTCAGATGGAGGAGGA | 443 | UCCUCCUCCAUCUGAGUC |
| 42 | 311 | 328 | ACTCAGATGGAGGAGGAT | 444 | AUCCUCCUCCAUCUGAGU |
| 43 | 312 | 329 | CTCAGATGGAGGAGGATA | 445 | UAUCCUCCUCCAUCUGAG |
| 44 | 313 | 330 | TCAGATGGAGGAGGATAT | 446 | AUAUCCUCCUCCAUCUGA |
| 46 | 315 | 332 | AGATGGAGGAGGATATTC | 448 | GAAUAUCCUCCUCCAUCU |
| 47 | 316 | 333 | GATGGAGGAGGATATTCT | 449 | AGAAUAUCCUCCUCCAUC |
| 49 | 319 | 336 | GGAGGAGGATATTCTGCA | 451 | UGCAGAAUAUCCUCCUCC |
| 50 | 321 | 338 | AGGAGGATATTCTGCAGC | 452 | GCUGCAGAAUAUCCUCCU |
| 52 | 379 | 396 | CCAGGCACAGAAGGTGCT | 454 | AGCACCUUCUGUGCCUGG |
| 55 | 413 | 430 | CGGCTAGAAGTCCAGCTG | 457 | CAGCUGGACUUCUAGCCG |
| 56 | 414 | 431 | GGCTAGAAGTCCAGCTGA | 458 | UCAGCUGGACUUCUAGCC |
| 57 | 415 | 432 | GCTAGAAGTCCAGCTGAG | 459 | CUCAGCUGGACUUCUAGC |
| 61 | 459 | 476 | GAGAATTTGAGGTCTTAA | 463 | UUAAGACCUCAAAUUCUC |
| 62 | 460 | 477 | AGAATTTGAGGTCTTAAA | 464 | UUUAAGACCUCAAAUUCU |
| 63 | 462 | 479 | AATTTGAGGTCTTAAAGG | 465 | CCUUUAAGACCUCAAAUU |
| 64 | 463 | 480 | ATTTGAGGTCTTAAAGGC | 466 | GCCUUUAAGACCUCAAAU |
| 65 | 464 | 481 | TTTGAGGTCTTAAAGGCT | 467 | AGCCUUUAAGACCUCAAA |
| 66 | 465 | 482 | TTGAGGTCTTAAAGGCTC | 468 | GAGCCUUUAAGACCUCAA |
| 67 | 466 | 483 | TGAGGTCTTAAAGGCTCA | 469 | UGAGCCUUUAAGACCUCA |
| 69 | 469 | 486 | GGTCTTAAAGGCTCACGC | 471 | GCGUGAGCCUUUAAGACC |
| 70 | 471 | 488 | TCTTAAAGGCTCACGCTG | 472 | CAGCGUGAGCCUUUAAGA |
| 71 | 472 | 489 | CTTAAAGGCTCACGCTGA | 473 | UCAGCGUGAGCCUUUAAG |
| 72 | 473 | 490 | TTAAAGGCTCACGCTGAC | 474 | GUCAGCGUGAGCCUUUAA |
| 73 | 474 | 491 | TAAAGGCTCACGCTGACA | 475 | UGUCAGCGUGAGCCUUUA |
| 74 | 475 | 492 | AAAGGCTCACGCTGACAA | 476 | UUGUCAGCGUGAGCCUUU |
| 77 | 479 | 496 | GCTCACGCTGACAAGCAG | 479 | CUGCUUGUCAGCGUGAGC |
| 88 | 530 | 547 | CAGCGGCAGAGGCGGGAG | 490 | CUCCCGCCUCUGCCGCUG |
| 89 | 534 | 551 | GGCAGAGGCGGGAGATGG | 491 | CCAUCUCCCGCCUCUGCC |
| 90 | 540 | 557 | GGCGGGAGATGGTGGCAC | 492 | GUGCCACCAUCUCCCGCC |
| 91 | 543 | 560 | GGGAGATGGTGGCACAGC | 493 | GCUGUGCCACCAUCUCCC |
| 93 | 553 | 570 | GGCACAGCAGCATCGGCT | 495 | AGCCGAUGCUGCUGUGCC |
| 97 | 559 | 576 | GCAGCATCGGCTGCGACA | 499 | UGUCGCAGCCGAUGCUGC |
| 98 | 560 | 577 | CAGCATCGGCTGGGACAG | 500 | CUGUCGCAGCCGAUGCUG |
| 99 | 561 | 578 | AGCATCGGCTGCGACAGA | 501 | UCUGUCGCAGCCGAUGCU |
| 100 | 562 | 579 | GCATCGGCTGCGACAGAT | 502 | AUCUGUCGCAGCCGAUGC |
| 101 | 563 | 580 | CATCGGCTGCGACAGATC | 503 | GAUCUGUCGCAGCCGAUG |
| 102 | 564 | 581 | ATCGGCTGCGACAGATCC | 504 | GGAUCUGUCGCAGCCGAU |
| 103 | 565 | 582 | TCGGCTGCGACAGATCCA | 505 | UGGAUCUGUCGCAGCCGA |
| 104 | 567 | 584 | GGCTGCGACAGATCCAGG | 506 | CCUGGAUCUGUCGCAGCC |
| 105 | 569 | 586 | CTGCGACAGATCCAGGAG | 507 | CUCCUGGAUCUGUCGCAG |
| 106 | 570 | 587 | TGCGACAGATCCAGGAGA | 508 | UCUCCUGGAUCUGUCGCA |
| 107 | 571 | 588 | GCGACAGATCCAGGAGAG | 509 | CUCUCCUGGAUCUGUCGC |

### EXAMPLE 2

An ANGPTL8 siRNA conjugated to a control delivery moiety (cntrl-GalNAc) comprising 3 GalNAc groups is compared to the same siRNA conjugated to a delivery moiety of Formula I, and compared to an siRNA lacking a 5′ phosphate on the antisense strand and assayed as described in this Example 2. The sense and antisense strands of the three siRNAs are shown below in Table 3; the three siRNAs each have one of the following pairs of sense and antisense strands, respectively: SEQ ID NOs: 381 and 397; or 382 and 398; or 366 and 372. Modifications are noted immediately prior to the corresponding modified nucleotide. P stands for phosphorylation, m for methylation of the OH group creating a 2′ methoxy modification on the ribose; f for a 2′ fluoro modification of the ribose, * for a phosphorothioate modification of the phosphodiester bond of the backbone).

TABLE 3

| | Sense | | |
|---|---|---|---|
| SEQ | or | | |
| ID | Anti- | Sense or Anti-Sense with | Delivery |
| NO | Sense | modifications (5′ to 3′) | Moiety |
| 381 | Sense | mC*mG*mAmGmAmAfUmUfUfGfA | Control |
| | | mGmGmUmCmUmUmAmA*mA*mG | GalNAc |
| 397 | Anti- | PmC*fU*mUmUmAfAmGmAmCmCm | |
| | sense | UmCmAfAmAfUmUmCmUmCmG* | |
| | | mG*mU | |
| 382 | Sense | mC*mG*mAmGmAmAfUmUfUfGfA | Formula |
| | | mGmGmUmCmUmUmAmA*mA*mG | I |
| 398 | Anti- | PmC*fU*mUmUmAfAmGmAmCmC | |
| | sense | mUmCmAfAmAfUmUmCmUmCmG* | |
| | | mG*mU | |
| 366 | Sense | mC*mG*mAmGmAmAfUmUfUfGf | Formula |
| | | AmGmGmUmCmUmUmAmA*mA*mG | I |
| 372 | Anti- | mC*fU*mUmUmAfAmGmAmCmC | |
| | sense | mUmCmAfAmAfUmUmCmUmCmG* | |
| | | mG*mU | |

Three assays are performed to analyze ANGPTL3/8 levels, triglyceride levels, and knockdown of ANGPTL8 expression as measured by mRNA levels (% KD), in order to compare the above siRNAs.

The conjugated siRNAs are tested in male transgenic mice for human cholesterol ester transfer protein (CETP) and apolipoprotein A1 (Taconic farms). Mice are dosed by retro-orbital injection with two adeno-associated virus (AAV) vectors. The first vector contains a plasmid with an albumin promoter and the coding sequence for human ANGPTL8 (NM_018687.7) SEQ ID NO:1. The second vector contains a mouse codon optimized sequence of human ANGPTL3 (NP_055310.1) SEQ ID NO:2. Animals are weighed and blood is collected from mice 3 to 5 weeks post AAV administration. This is considered the pre-study bleed. Serum is prepared from blood and triglycerides are measured utilizing a COBAS clinical chemistry analyzer (Roche) and ANGPTL3/8 protein levels are measured by ELISA (Meso Scale Diagnostics). Mice are assigned to groups with similar body weight, serum triglyceride and ANGPTL3/8 levels (n=6). Blood is collected on study day 0 and this collection is considered the baseline. Either PBS or test siRNAs, at doses of 3 and 10 mg/kg are administered subcutaneously to mice on study day 0. Blood is collected from mice 1 and 2 weeks post siRNA administration under isoflurane anesthesia. Serum is prepared from blood and triglycerides are measured utilizing a COBAS clinical chemistry analyzer (Roche). Serum ANGPTL3/8 levels are measured by an ELISA (Meso Scale Diagnostics). Triglyceride as a percent change from baseline at 1 weeks is calculated as ((triglyceride at one weeks minus triglyceride at baseline)/(triglyceride at baseline))*100. Triglyceride as a percent change from baseline at 2 weeks is calculated similarly. ANGPTL3/8 as a percent change from baseline at 1 week is calculated as ((ANGPTL3/8 at one week minus ANGPTL3/8 at baseline)/(ANGPTL3/8 at baseline))*100. ANGPTL3/8 as a percent change from baseline at 2 weeks is calculated similarly. Serum triglyceride and ANGPTL3/8 data is analyzed for a statistically significant difference from the PBS group at corresponding timepoint using ANOVA and Dunnett's method where p<0.05 was considered statistically significant (SAS Institute).

For measuring in vitro knockdown, mice are euthanized under isoflurane anesthesia two weeks post subcutaneous injection. Liver is collected from the mice and frozen in liquid nitrogen. Livers are homogenized in TriZol (Invitrogen) using Lysing Matrix D bead tubes on a FastPrep-24 (MP Bio), and RNA is purified and resuspended in nuclease free water. The RNA is quantitated on the NanoDrop (ThermoFisher). Equal amounts of RNA are reverse transcribed to cDNA using High-Capacity cDNA Reverse Transcription kit (Life Technologies) on a ProFlex Thermocycler (Thermo Fisher). Thermocycler settings are 25° C. for 10 min, 37° C. for 2 hrs, then 85° C. for 5 min. Template cDNA is combined with Taqman Universal Master Mix and Assays on Demand primer/probesets and RT-PCR is performed on QuantStudio 7 Flex Real-Time PCR system (Applied Biosystems) with the following parameters: 50° C. for 2 min, 95° C. for 10 min then 40 cycles of 95° C. for 15 sec and 60° C. for 1 min. Fold changes (FC) are calculated as follows: the CT value of mouse Rp1p0 is subtracted from CT value of human ANGPTL8 to obtain the delta CT value. The delta CT value is calculated by subtracting the average delta CT value of the untreated sample (PBS control) for each gene from the delta CT value of each test sample. Fold change is calculated by taking the log base 2 of the negative delta CT value. Percent knock down (% KD) is calculated by subtracting FC from one and multiplying by 100. Data is shown in Table 4. The two siRNAs comprising the delivery moiety of Formula I perform better than the control delivery moiety, and demonstrate lower levels of ANGPTL8, lower triglyceride levels, and a higher percent knockdown of ANGPTL8 mRNA.

TABLE 4

| | Anti- | | | | | | |
|---|---|---|---|---|---|---|---|
| Sense | Sense | | | | | | % KD |
| SEQ | SEQ | Dose | 1 week | 2 week | 1 week | 2 week | ANGPTL8 |
| ID NO | ID NO | (mg/kg) | ANGPTL3/8 | ANGPTL3/8 | TRIG | TRIG | mRNA |
| 381 | 397 | 3 | −57* | −71* | −44* | −49* | 84* |
| | | 10 | −87* | −93* | −73* | −77* | 97* |
| 382 | 398 | 3 | −70* | −82* | −56* | −61* | 97* |
| | | 10 | −93* | −96* | −74* | −77* | 96* |
| 366 | 372 | 3 | −81* | −87* | −65* | −61* | 96* |
| | | 10 | −89* | −94* | −71* | −73* | 98* |

*indicates p < 0.05 ANOVA with Dunnetts

### EXAMPLE 3

Exemplary siRNAs that are complementary to the above 18mer regions of the ANGPTL8 transcript NM (SEQ ID NO:1) (Table 1), are designed and shown in the sequence listing below by the underlying nucleotide sequence, where each row represents an siRNA having the given sense and antisense strand. As shown, the underlying sense and antisense RNA oligonucleotides strands are between 18 and 23 nucleotides in length and with optional overhangs of 1 to 5 ribonucleotides. The underlying nucleotide sequence shown is modified, with 1-10 fluoro additions at the 2′ position of ribose, and the remaining residues are methylated at the 2′position of ribose (creating a 2′ methoxy modification). Some antisense strands are phosphorylated at the 5′ position. Each siRNA is conjugated to a delivery moiety comprising 3 GalNAc groups; select delivery moieties comprise Formula I while others comprise a control moiety. One or more phosphodiester bonds are present at the 5′ and 3′ ends. A control GalNAc is attached at the 3′ end of the sense strand. Subsets of these siRNAs are tested in an in vivo knockdown assay.

For the in vivo knockdown assay, select siRNAs are tested in male C57b1/6 mice (Taconic farms). Mice are dosed by retro-orbital injection with an adeno-associated virus (AAV) vector containing a plasmid with an albumin promoter and the coding sequence for human ANGPTL8 (SEQ ID NO:1 (NCBI Reference Sequence NM_018687.7) (Vector BioLabs). Mice are weighed two weeks post AAV administration. Mice are assigned to groups with similar body weight (n=5). Either PBS or test siRNA, at a dose of 10 mg/kg, is administered subcutaneously to mice. Seven days post subcutaneous injection mice are euthanized under isoflurane anesthesia. Liver is collected from the mice and frozen in liquid nitrogen. RNA is isolated and purified from the collected liver and used for cDNA synthesis and quantified by RT PCR.

Fold changes (FC) are calculated as follows: the CT value of mouse Rp1p0 is subtracted from CT value of human ANGPTL8 to obtain the delta CT value. Relative amount is calculated by taking the log base 2 of the negative delta delta CT value. Fold change is calculated by dividing the relative amount of each sample by the average of the control group. Percent knock down (% KD) is calculated by subtracting FC from one and multiplying by 100. Data is shown in Table 5.

TABLE 5

| siRNA | Sense | Anti-Sense | |
|---|---|---|---|
| (Row Number | SEQ ID | SEQ ID | |
| from Table 11) | NO | NO | % KD |
| 1A | 124 | 231 | 64.9 |
| 3C | 126 | 233 | 63.6 |
| 5E | 128 | 235 | 80.6 |
| 7G | 130 | 237 | 59.2 |
| 10J | 133 | 240 | 71.1 |
| 40NN | 151 | 270 | 72.7 |
| 43QQ | 154 | 273 | 55.7 |
| 44RR | 155 | 274 | 51.1 |
| 49WW | 160 | 279 | 67.6 |
| 52ZZ | 163 | 282 | 75.5 |
| 62D3 | 167 | 286 | 73.7 |
| 64F3 | 169 | 288 | 72.5 |
| 91P# | 177 | 292 | 50.2 |
| 98V3 | 183 | 298 | 75.2 |
| 116f4 | 193 | 308 | 53.2 |
| 118h4 | 195 | 310 | 71.8 |
| 150H5 | 200 | 336 | 57.1 |
| 161S5 | 217 | 347 | 81.4 |
| 162TS | 218 | 348 | 69.2 |
| 165W5 | 221 | 351 | 72.2 |

The same procedures described above for the 10 mg/kg dose are performed at a dose of 3 mg/kg for measuring the in vivo knockdown of the following siRNAs in Table 6. (each siRNA has the sense and antisense strands in vertical order, where the first tested siRNA comprises the first two rows of the table, SEQ ID NOs 373 and 389, the next siRNA comprises the 3^{rd} and 4^{th} rows of the table, SEQ ID NOs 374 and 390, and so forth; a control GalNAc is attached at the 3′ end of the sense strand for each siRNA.) Abbreviations for modifications are the same as shown above in Example 3. Results for select RNAs are shown below in Table 7.

TABLE 6

| | Sense | |
|---|---|---|
| SEQ | or | |
| ID | Anti- | Sense or Anti-Sense with |
| NO | Sense | modifications (5′ to 3′) |
| 373 | Sense | mU*mC*mAmGmAmUmGmGfAfGfGmAmGmGmAmUmAmU |
| | | mUmCmU |
| 389 | Anti- | PmA*fG*mAmAmUfAmUmCmCmUmCmCmUfCmCfAmU |
| | Sense | mCmUmGmA*mG*mU |
| 374 | Sense | mG*mC*mAmGmAmUfGmGfAfGfGmAmGmGmAmUmAmU |
| | | mU*mC*mA |
| 390 | Anti- | PmU*fG*mAmAmUfAmUmCmCmUmCmCmUfCmCfAmU |
| | Sense | mCmUmGmC*mG*mU |
| 375 | Sense | mU*mC*mAmGmAmUfGmGfAfGfGmAmGmGmAmUmAmU |
| | | mUmCmU |
| 391 | Anti- | PmA*fG*mAmAmUfAmUmCmCmUmCmCmUfCmCfAmU |
| | Sense | mCmUmGmA*mG*mU |
| 376 | Sense | mU*mC*mAmGmAmUfGmGfAfGfGmAmGmGmAmUmAmU |
| | | mU*mC*mC |
| 392 | Anti- | PmG*fG*mAmAmUfAmUmCmCmUmCmCmUfCmCfAmU |
| | Sense | mCmUmGmA*mG*mU |
| 377 | Sense | mU*mC*mAmGmAmUfGmGfAfGfGmAmGmGmAmUmAmU |
| | | mUmCmU |
| 393 | Anti- | PmA*fG*mAmAmUmAmUmCmCmUmCmCmUfCmCmAmU |
| | Sense | mCmUmGmA*mG*mU |
| 378 | Sense | mC*mA*mGmAmUmGfGmAfGfGfAmGmGmAmUmAmUmU |
| | | mC*mU*mA |
| 394 | Anti- | PmU*fA*mGmAmAfUmAmUmCmCmUmCmCfUmCfCmA |
| | Sense | mUmCmUmG*mA*mG |
| 379 | Sense | mA*mU*mGmGmAmGfGmAfGfGfAmUmAmUmUmCmUmG |
| | | mC*mA*mA |
| 395 | Anti- | PmU*fU*mGmCmAfGmAmAmUmAmUmCmCfUmCfCmU |
| | Sense | mCmCmAmU*mC*mU |
| 380 | Sense | mC*mC*mGmAmGmAfAmUfUfUfGmAmGmGmUmCmUmU |
| | | mA*mA*mA |
| 396 | Anti- | PmU*fU*mUmAmAfGmAmCmCmUmCmAmAfAmUfUmC |
| | Sense | mUmCmGmG*mU*mA |
| 381 | Sense | mC*mG*mAmGmAmAfUmUfUfGfAmGmGmUmCmUmUmA |
| | | mA*mA*mG |
| 397 | Anti- | PmC*fU*mUmUmAfAmGmAmCmCmUmCmAfAmAfUmU |
| | Sense | mCmUmCmG*mG*mU |
| 382 | Sense | mC*mG*mAmGmAmAfUmUfUfGfAmGmGmUmCmUmUmA |
| | | mA*mA*mG |
| 398 | Anti- | PmC*fU*mUmUmAfAmGmAmCmCmUmCmAfAmAfUmU |
| | Sense | mCmUmCmG*mG*mU |
| 383 | Sense | mG*mG*mAmGmAmAfUmUfUfGfAmGmGmUmCmUmUmA |
| | | mA*mA*mA |
| 399 | Anti- | PmU*fU*mUmUmAfAmGmAmCmCmUmCmAfAmAfUmU |
| | Sense | mCmUmCmC*mG*mU |
| 384 | Sense | mC*mG*mAmGmAmAfUmUfUfGfAmGmGmUmCmUmUmA |
| | | mA*mA*mA |
| 400 | Anti- | PmU*fU*mUmUmAfAmGmAmCmCmUmCmAfAmAfUmU |
| | Sense | mCmUmCmG*mG*mU |
| 385 | Sense | mC*mG*mAmGmAmAfUmUfUfGfAmGmGmUmCmUmUmA |
| | | mAmAmG |
| 401 | Anti- | PmC*fU*mUmUmAfAmGmAmCmCmUmCmAfAmAfUmU |
| | Sense | mCmUmCmG*mG*mU |
| 386 | Sense | mC*mG*mAmGmAmAmUmUfUfGfAmGmGmUmCmUmUmA |
| | | mAmAmG |
| 402 | Anti- | PmC*fU*mUmUmAfAmGmAmCmCmUmCmAfAmAfUmU |
| | Sense | mCmUmCmG*mG*mU |
| 387 | Sense | mC*mG*mAmGmAmAfUmUfUfGfAmGmGmUmCmUmUmA |
| | | mAmAmG |
| 403 | Anti- | PmC*fU*mUmUmAmAmGmAmCmCmUmCmAfAmAmUmU |
| | Sense | mCmUmCmG*mG*mU |
| 388 | Sense | mC*mG*mAmGmAmAmUmUfUfGfAmGmGmUmCmUmUmA |
| | | mAmAmG |
| 404 | Anti- | PmC*fU*mUmUmAmAmGmAmCmCmUmCmAfAmAmUmU |
| | Sense | mCmUmCmG*mG*mU |

TABLE 7

| Sense | Anti-Sense | % KD |
|---|---|---|
| SEQ ID NO | SEQ ID NO | ANGPTL8 mRNA |
| 381 | 397 | 49.8 |
| 380 | 396 | 69.2 |
| 376 | 392 | 44.3 |
| 388 | 404 | 38.9 |
| 374 | 390 | 44.7 |
| 387 | 403 | 37.9 |
| 377 | 393 | 39.1 |
| 383 | 399 | 44.9 |
| 384 | 400 | 57.8 |
| 385 | 401 | 53.8 |
| 379 | 395 | 65.1 |
| 373 | 389 | 51.8 |
| 375 | 391 | 52.7 |
| 386 | 402 | 58.9 |
| 378 | 394 | 56.8 |

### EXAMPLE 5

GalNAc-siRNAs are tested in male transgenic mice with human cholesterol ester transfer protein (CETP) and apolipoprotein A1 (Taconic fars). The siRNAs are divided and tested in 3 studies (n=2, n=2, and n=2). Mice are dosed by retro-orbital injection with two adeno-associated virus (AAV) vectors. One vector contains a plasmid with an albumin promoter and the coding sequence for human ANGPTL8 (SEQ ID NO:1). The second vector contains a mouse codon optimized sequence of human ANGPTL3 (SEQ ID NO:2)(NP_055310.1). A Baseline blood sample is collected from mice 4 to 6.5 weeks post AAV administration. Serum is prepared from blood and triglycerides are measured utilizing a COBAS clinical chemistry analyzer (Roche) and ANGPTL3/8 is measured by ELISA (Meso Scale Diagnostics). Mice are assigned to groups with similar serum triglyceride and ANGPTL3/8 levels. siRNAs are designed as shown in Table 8; each siRNA is conjugated to a delivery moiety of Formula I. Either PBS or the siRNAs, at doses of 0.3, 1, 3 and 10 mg/kg are administered subcutaneously to mice. Blood is collected from mice 3, 6, and 9 weeks post siRNA administration under isoflurane anesthesia. Serum is prepared from blood and triglycerides are measured utilizing a COBAS clinical chemistry analyzer (Roche).

Triglyceride as a percent change from baseline at 3 weeks is calculated as ((triglyceride at three weeks minus triglyceride at baseline)/(triglyceride at baseline))*100. Triglyceride as a percent change from baseline at 6 and 9 week is calculated similarly. Triglyceride Data was analyzed for a statistically significant difference from the PBS group at corresponding timepoint using ANOVA and Dunnett's method where p<0.05 was considered statistically significant (SAS Institute) Data is shown in Table 9.

The corresponding in vitro percent knockdown at 1000 nM for each of the molecules is shown in Table 10.

TABLE 8

| | | Anti- | |
|---|---|---|---|
| Sense | Sense strand | Sense | Antisense |
| SEQ | sequence with | SEQ | strand with |
| ID | modifications | ID | modifications |
| NO | (5′ to 3′) | NO | (5′ to 3′) |
| 361 | mA*mU*mGmGmAmG | 367 | [Phos]mU*fU*mGmCmA |
| | fGmAfGfGfAmUmA | | fGmAmAmUmAmUmCmCfU |
| | mUmUmCmUmGmC* | | mCfCmUmCmCmAmU*mC* |
| | mA*mA | | mU |
| 362 | mC*mC*mGmAmGmA | 368 | [Phos]mU*fU*mUmAmA |
| | fAmUfUfUfGmAmG | | fGmAmCmCmUmCmAmAfA |
| | mGmUmCmUmUmA* | | mUfUmCmUmCmGmG*mU* |
| | mA*mA | | mA |
| 363 | mA*mU*mGmGmAmG | 369 | mU*fU*mGmCmAfGmAmA |
| | fGmAfGfGfAmUmA | | mUmAmUmCmCfUmCfCmU |
| | mUmUmCmUmGmC* | | mCmCmAmU*mC*mU |
| | mA*mA | | |
| 364 | mG*mG*mUmCmUmU | 370 | mU*fC*mAmGmCfGmUmG |
| | fAmAfAfGfGmCmU | | mAmGmCmCmUfUmUfAmA |
| | mCmAmCmGmCmU* | | mGmAmCmC*mU*mC |
| | mG*mA | | |
| 365 | mA*mU*mGmGmAmG | 371 | mC*fU*mGmCmAfGmAmA |
| | fGmAfGfGfAmUmA | | mUmAmUmCmCfUmCfCmU |
| | mUmUmCmUmGmC* | | mCmCmAmU*mC*mU |
| | mA*mG | | |
| 366 | mC*mG*mAmGmAmA | 372 | mC*fU*mUmUmAfAmGmA |
| | fUmUfUfGfAmGmG | | mCmCmUmCmAfAmAfUmU |
| | mUmCmUmUmAmA* | | mCmUmCmG*mG*mU |
| | mA*mG | | |

TABLE 9

| | | SEQ ID | | | | | | |
|---|---|---|---|---|---|---|---|---|
| | SEQ ID | NO of | | | | | | |
| | NO of | Anti- | Dose | 3 week | 6 week | 9 week | 12 week | 15 week |
| siRNA | Sense | Sense | (mg/kg) | % chg | % chg | % chg | % chg | % chg |
| i | 361 | 367 | 0.3 | 5 | −4 | 10 | −9 | −13 |
| | | | 1 | −29* | −21* | −20* | −38* | −32 |
| | | | 3 | −62* | −56* | −45* | −50* | −50* |
| | | | 10 | −86* | −85* | −78* | −75* | −74* |
| ii | 362 | 368 | 0.3 | −24* | −10 | −14 | −33* | −19 |
| | | | 1 | −44* | −31* | −13 | −27* | −25 |
| | | | 3 | −53* | −48* | −28* | −38* | −30 |
| | | | 10 | −65* | −64* | −54* | −51* | −35 |
| iii | 365 | 371 | 0.3 | −27 | −27 | −24 | −41 | −35 |
| | | | 1 | −48* | −39 | −38 | −45 | −41 |
| | | | 3 | −81* | −72* | −72* | −73* | −64* |
| | | | 10 | −87* | −84* | −80* | −83* | −79* |
| iv | 366 | 372 | 0.3 | −24 | −33 | −27 | −42 | −42 |
| | | | 1 | −57* | −51* | −46* | −56* | −48 |
| | | | 3 | −65* | −61* | −48* | −52* | −43 |
| | | | 10 | −76* | −73* | −60* | −61* | −55 |
| v | 363 | 369 | 0.3 | −4 | −8 | −4 | −2 | −17 |
| | | | 1 | −51* | −37* | −27* | −8 | −31 |
| | | | 3 | −73* | −66* | −55* | −60* | −50* |
| | | | 10 | −88* | −86* | −86* | −81* | −79* |
| vi | 364 | 370 | 0.3 | 21 | 9 | 4 | −5 | −19 |
| | | | 1 | −23* | −18 | −16 | −11 | −30 |
| | | | 3 | −42* | −34* | −23 | −18 | −25 |
| | | | 10 | −72* | −63* | −54* | −42* | −50* |

TABLE 10

| | Sense SEQ | Antisense SEQ | % KD at |
|---|---|---|---|
| siRNA | ID NO | ID NO | 1000 nM |
| i | 361 | 367 | 92.1 |
| ii | 362 | 368 | 93.7 |
| iii | 363 | 369 | 92.4 |
| iv | 364 | 370 | 77.4 |
| v | 365 | 371 | 81.0 |
| vi | 366 | 372 | 93.4 |

TABLE 11

| | | Sense | | Anti- |
|---|---|---|---|---|
| | | SEQ | | Sense |
| | | ID | | SEQ |
| Row | Sense Sequence | NO | Anti-Sense Sequence | ID NO |
| 1A | GGAGCUGACCCUGCUCUUCCA | 124 | UGGAAGAGCAGGGUCAGCUCCUC | 231 |
| 2B | CCACGGAGGGACGGCUGACAA | 125 | UUGUCAGCCGUCCCUCCGUGGUC | 232 |
| 3C | ACGGCUGACAAAGGCCAGGAA | 126 | UUCCUGGCCUUUGUCAGCCGUCC | 233 |
| 4D | GGCUGACAAAGGCCAGGAACA | 127 | UGUUCCUGGCCUUUGUCAGCCGU | 234 |
| 5E | CAGGAACAGCCUGGGUCUCUA | 128 | UAGAGACCCAGGCUGUUCCUGGC | 235 |
| 6F | CAGGAACAGCCUGGGUCUCUC | 129 | GAGAGACCCAGGCUGUUCCUGGC | 236 |
| 7G | AGGAACAGCCUGGGUCUCUAU | 130 | AUAGAGACCCAGGCUGUUCCUGG | 237 |
| 8H | AGGAACAGCCUGGGUCUCUAA | 131 | UUAGAGACCCAGGCUGUUCCUGG | 238 |
| 9I | GCCUGGGUCUCUAUGGCCGCA | 132 | UGCGGCCAUAGAGACCCAGGCUG | 239 |
| 10J | CUGGGUCUCUAUGGCCGCACA | 133 | UGUGCGGCCAUAGAGACCCAGGC | 240 |
| 11K | CUUGGUCUCUAUGGCCGCACA | 134 | UGUGCGGCCAUAGAGACCAAGGC | 241 |
| 12L | CUGGGUCUCUAUGGCCGCACA | 133 | UGUGCGGCCAUAGAGACCCAGUG | 242 |
| 13M | CUGGGUCUCUAUGGCCGCACA | 133 | UGUGCGGCCAUAGAGACCCAGGC | 243 |
| 14N | CUGGGUCUCUAUGGCCGCACA | 133 | UGUGCGGCCAUAGAGACCCAGUU | 244 |
| 15O | CUGGGUCUCUAUGGCCGCACA | 133 | UGUGCGGCCAUAGAGACCCAGAG | 245 |
| 16P | CUGGGUCUCUAUGGCCGCACA | 133 | UGUGCGGCCAUAGAGACCCAGAU | 246 |
| 17Q | CUGGGUCUCUAUGGCCGCACA | 133 | UGUGCGGCCAUAGAGACCCAGGC | 247 |
| 18R | CUGGGUCUCUAUGGCCGCACA | 133 | UGUGCGGCCAUAGAGACCCAGAA | 248 |
| 19S | CUAGGUCUCUAUGGCCGCACA | 135 | UGUGCGGCCAUAGAGACCCAGGC | 249 |
| 20T | CUGGGUCUCUAUGGCCGCACA | 133 | CGCGCGGCCACAGAGACCCAGCG | 250 |
| 21U | CAAGGUCUCUAUGGCCGCACA | 136 | CGCGCGGCCACAGAGACCCCGGC | 251 |
| 22V | CUGGGUCUCUAUGGCCGCACA | 133 | CGCGCGGCCACAGAGACCCAGCA | 252 |
| 23W | CUGGGUCUCUAUGGCCGCACA | 133 | CGCGCGGCCACAGAGACCCAGAC | 253 |
| 24X | CUGGGUCUCUAUGGCCGCACA | 133 | CGCGCGGCCACAGAGACCCAGCC | 254 |
| 25Y | CCCGGUCUCUAUGGCCGCACA | 137 | CGCGCGGCCACAGAGACCGGGGC | 255 |
| 26Z | CUGGGUCUCUAUGGCCGCACA | 133 | CGCGCGGCCACAGAGACCCAGCA | 256 |
| 27AA | CAUGGUCUCUAUGGCCGCACA | 138 | CGCGCGGCCACAGAGACCACGGC | 257 |
| 28BB | CAGGGUCUCUAUGGCCGCACA | 139 | CGCGCGGCCACAGAGACCCCGGC | 258 |
| 29CC | CGAGGUCUCUAUGGCCGCACA | 140 | CGCGCGGCCACAGAGACCCCGGC | 259 |
| 30DD | CGGGGUCUCUAUGGCCGCACA | 141 | CGCGCGGCCACAGAGACCCCGGC | 260 |
| 31EE | CGAGGUCUCUAUGGCCGCACA | 142 | CGCGCGGCCACAGAGACCCCGGC | 261 |
| 32FF | CCGGGAUGCAGCCCAGGAACU | 143 | AGCCCCCGGGCCGCACCCCGGCC | 262 |
| 33GG | CGGGAUGCAGCCCAGGAACUU | 144 | AAGCCCCCGGGCCGCACCCCGGC | 263 |
| 34HH | AGCCCAGGAACUUCGGGCAAA | 145 | CCCGCCCGAAGCCCCCGGGCCGC | 264 |
| 35II | GCCCAGGAACUUCGGGCAAGA | 146 | UCUUGCCCGAAGUUCCUGGGCUG | 265 |
| 36JJ | CAGGAACUUCGGGCAAGCCUA | 147 | CAGGCCCGCCCGAAGCCCCCGGG | 266 |
| 37KK | AGGAACUUCGGGCAAGCCUGA | 148 | UCAGGCUUGCCCGAAGUUCCUGG | 267 |
| 38LL | AACUUCGGGCAAGCCUGUUGA | 149 | CCAACAGGCCCGCCCGAAGCCCC | 268 |
| 39MM | ACUUCGGGCAAGCCUGUUGGA | 150 | UCCAACAGGCUUGCCCGAAGUUC | 269 |
| 40NN | UUCGGGCAAGCCUGUUGGAGA | 151 | UCUCCAACAGGCUUGCCCGAAGU | 270 |
| 41OO | GGCAAGCCUGUUGGAGACUCA | 152 | UGAGUCUCCAACAGGCUUGCCCG | 271 |
| 42PP | CAAGCCUGUUGGAGACUCAGA | 153 | UCUGAGUCUCCAACAGGCUUGCC | 272 |
| 43QQ | AAGCCUGUUGGAGACUCAGAU | 154 | ACCCGAGCCCCCAACAGGCCCGC | 273 |
| 44RR | CCUGUUGGAGACUCAGAUGGA | 155 | UCCAUCUGAGUCUCCAACAGGCU | 274 |
| 45SS | CUGUUGGAGACUCAGAUGGAA | 156 | UUCCAUCUGAGUCUCCAACAGGC | 275 |
| 46TT | GGAGACUCAGAUGGAGGAGGA | 157 | UCCUCCUCCAUCUGAGUCUCCAA | 276 |
| 47UU | GAGACUCAGAUGGAGGAGGAU | 158 | AUCCUCCUCCAUCUGAGUCUCCA | 277 |
| 48VV | AGACUCAGAUGGAGGAGGAUA | 159 | UAUCCUCCUCCAUCUGAGUCUCC | 278 |
| 49WW | GACUCAGAUGGAGGAGGAUAU | 160 | AUAUCCUCCUCCAUCUGAGUCUC | 279 |
| 50XX | ACUCAGAUGGAGGAGGAUAUU | 161 | AAUAUCCUCCUCCAUCUGAGUCU | 280 |
| 51YY | ACUCAGAUGGAGGAGGAUAUA | 162 | UAUAUCCUCCUCCAUCUGAGUCU | 281 |
| 52ZZ | UCAGAUGGAGGAGGAUAUUCU | 163 | AGAAUAUCCUCCUCCAUCUGAGU | 282 |
| 54A3 | GCAGAUGGAGGAGGAUAUUCA | 164 | UGAAUAUCCUCCUCCAUCUGCGU | 283 |
| 56B3 | UCAGAUGGAGGAGGAUAUUCA | 165 | UGAAUAUCCUCCUCCAUCUGAGU | 284 |
| 57C3 | UCAGAUGGAGGAGGAUAUUCC | 166 | GGAAUAUCCUCCUCCAUCUGAGU | 285 |
| 62D3 | CAGAUGGAGGAGGAUAUUCUG | 167 | CAGAAUAUCCUCCUCCAUCUGAG | 286 |
| 63E3 | CAGAUGGAGGAGGAUAUUCUA | 168 | UAGAAUAUCCUCCUCCAUCUGAG | 287 |
| 64F3 | AUGGAGGAGGAUAUUCUGCAG | 169 | CUGCAGAAUAUCCUCCUCCAUCU | 288 |
| 65G3 | AUGGAGGAGGAUAUUCUGCAA | 170 | UUGCAGAAUAUCCUCCUCCAUCU | 289 |
| 69H3 | AUGGAGGAGGCUAUUCUGCAG | 171 | CUGCAGAAUAUCCUCCUCCAUCU | 288 |
| 74I3 | AUGGAGGAGGUUAUUCUGCAG | 172 | CUGCAGAAUAUCCUCCUCCAUCU | 288 |
| 80J3 | AUGGAGGAGGAUAUUCUGCAG | 169 | CUGCAGAAUAUCCUCCUCCAUCUTTT | 290 |
| 81K3 | AUGGAGGAGGGUAUUCUGCAG | 173 | CUGCAGAAUAUCCUCCUCCAUCU | 288 |
| 82L3 | AUGGAGGAGGAUAUUCUGCAG | 169 | CUGCAGAAUAUCCUCCUCCAUCUTT | 291 |
| 86M3 | AGAUGGAGGAGGAUAUUCUGCAG | 174 | CUGCAGAAUAUCCUCCUCCAUCU | 288 |
| 89N3 | CAGAUGGAGGAGGAUAUUCUGCAG | 175 | CUGCAGAAUAUCCUCCUCCAUCU | 288 |
| 90O3 | CCAGAUGGAGGAGGAUAUUCUGCAG | 176 | CUGCAGAAUAUCCUCCUCCAUCU | 288 |
| 91P3 | GGAGGAGGAUAUUCUGCAGCU | 177 | AGCUGCAGAAUAUCCUCCUCCAU | 292 |
| 92Q3 | GCCCAGGCACAGAAGGUGCUA | 178 | UAGCACCUUCUGUGCCUGGGCCA | 293 |
| 93R3 | AGCGGCUAGAAGUCCAGCUGA | 179 | UCAGCUGGACUUCUAGCCGCUGC | 294 |
| 94S3 | GCGGCUAGAAGUCCAGCUGAA | 180 | UUCAGCUGGACUUCUAGCCGCUG | 295 |
| 95T3 | CGGCUAGAAGUCCAGCUGAGA | 181 | UCUCAGCUGGACUUCUAGCCGCU | 296 |
| 96U3 | CCGAGAAUUUGAGGUCUUAAA | 182 | UUUAAGACCUCAAAUUCUCGGUA | 297 |
| 98V3 | CGAGAAUUUGAGGUCUUAAAG | 183 | CUUUAAGACCUCAAAUUCUCGGU | 298 |
| 99W3 | GGAGAAUUUGAGGUCUUAAAA | 184 | UUUUAAGACCUCAAAUUCUCCGU | 299 |
| 100X3 | CGAGAAUUUGAGGUCUUAAAA | 185 | UUUUAAGACCUCAAAUUCUCGGU | 300 |
| 109Y3 | AGAAUUUGAGGUCUUAAAGGA | 186 | UCCUUUAAGACCUCAAAUUCUCG | 301 |
| 110Z3 | GAAUUUGAGGUCUUAAAGGCA | 187 | UGCCUUUAAGACCUCAAAUUCUC | 302 |
| 111a4 | AAUUUGAGGUCUUAAAGGCUA | 188 | UAGCCUUUAAGACCUCAAAUUCU | 303 |
| 112b4 | AUUUGAGGUCUUAAAGGCUCA | 189 | UGAGCCUUUAAGACCUCAAAUUC | 304 |
| 113c4 | AUCUGAGGUCUUAAAGGCUCA | 190 | UGAGCCUUUAAGACCUCAGAUUC | 305 |
| 114d4 | ACCUGAGGUCUUAAAGGCUCA | 191 | UGAGCCUUUAAGACCUCAGGUUC | 306 |
| 115e4 | UUUGAGGUCUUAAAGGCUCAA | 192 | UUGAGCCUUUAAGACCUCAAAUU | 307 |
| 116f4 | GAGGUCUUAAAGGCUCACGCU | 193 | AGCGUGAGCCUUUAAGACCUCAA | 308 |
| 117g4 | GAGGUCUUAAAGGCUCACGCA | 194 | UGCGUGAGCCUUUAAGACCUCAA | 309 |
| 118h4 | GGUCUUAAAGGCUCACGCUGA | 195 | UCAGCGUGAGCCUUUAAGACCUC | 310 |
| 124i4 | UGUCUUAAAGGCUCACGCUGC | 196 | GCAGCGUGAGCCUUUAAGACAUC | 311 |
| 126j4 | GUCUUAAAGGCUCACGCUGAA | 197 | UUCAGCGUGAGCCUUUAAGACCU | 312 |
| 127k4 | UCUUAAAGGCUCACGCUGACA | 198 | UGUCAGCGUGAGCCUUUAAGACC | 313 |
| 128l4 | CCGAAAGGCUCACGCUGACAA | 199 | UUGUCAGCGUGAGCCUUUCGGAC | 314 |
| 129m4 | CUUAAAGGCUCACGCUGACAA | 200 | UUGUCAGCGUGAGCCUUUAAGAA | 315 |
| 130n4 | CACAAAGGCUCACGCUGACAA | 201 | UUGUCAGCGUGAGCCUUUGUGAC | 316 |
| 131o4 | CUUAAAGGCUCACGCUGACAA | 200 | UUGUCAGCGUGAGCCUUUAAGGU | 317 |
| 132p4 | CCUAAAGGCUCACGCUGACAA | 202 | UUGUCAGCGUGAGCCUUUAGGAC | 318 |
| 133q4 | CUUAAAGGCUCACGCUGACAA | 200 | UUGUCAGCGUGAGCCUUUAAGCA | 319 |
| 134r4 | CUUAAAGGCUCACGCUGACAA | 200 | UUGUCAGCGUGAGCCUUUAAGAG | 320 |
| 135s4 | CUGAAAGGCUCACGCUGACAA | 203 | UUGUCAGCGUGAGCCUUUCAGAC | 321 |
| 136t4 | CUUAAAGGCUCACGCUGACAA | 200 | UUGUCAGCGUGAGCCUUUAAGGG | 322 |
| 137u4 | CUCAAAGGCUCACGCUGACAA | 204 | UUGUCAGCGUGAGCCUUUGAGAC | 323 |
| 138v4 | CCAAAAGGCUCACGCUGACAA | 205 | UUGUCAGCGUGAGCCUUUUGGAC | 324 |
| 139w4 | CUUAAAGGCUCACGCUGACAA | 200 | UUGUCAGCGUGAGCCUUUAAGUA | 325 |
| 140x4 | CUUAAAGGCUCACGCUGACAA | 200 | UUGUCAGCGUGAGCCUUUAAGGG | 326 |
| 141y4 | CUUAAAGGCUCACGCUGACAA | 200 | UUGUCAGCGUGAGCCUUUAAGGU | 327 |
| 142z4 | CUUAAAGGCUCACGCUGACAA | 200 | UUGUCAGCGUGAGCCUUUAAGGA | 328 |
| 143A5 | CUUAAAGGCUCACGCUGACAA | 200 | UUGUCAGCGUGAGCCUUUAAGUG | 329 |
| 144B5 | CAAAAAGGCUCACGCUGACAA | 206 | UUGUCAGCGUGAGCCUUUUUGAC | 330 |
| 145C5 | CGCAAAGGCUCACGCUGACAA | 207 | UUGUCAGCGUGAGCCUUUGCGAC | 331 |
| 146D5 | CGAAAAGGCUCACGCUGACAA | 208 | UUGUCAGCGUGAGCCUUUUGGAC | 332 |
| 147E5 | CUUAAAGGCUCACGCUGACAA | 200 | UUGUCAGCGUGAGCCUUUAAGGG | 333 |
| 148F5 | CAGAAAGGCUCACGCUGACAA | 209 | UUGUCAGCGUGAGCCUUUGUGAC | 334 |
| 149G5 | CCCAAAGGCUCACGCUGACAA | 210 | UUGUCAGCGUGAGCCUUUGGGAC | 335 |
| 150H5 | CUUAAAGGCUCACGCUGACAA | 200 | UUGUCAGCGUGAGCCUUUAAGAG | 336 |
| 151I5 | CUUAAAGGCUCACGCUGACAA | 200 | UUGUCAGCGUGAGCCUUUAAGCC | 337 |
| 152J5 | CUUAAAGGCUCACGCUGACAA | 200 | UUGUCAGCGUGAGCCUUUAAGAU | 338 |
| 153K5 | CGUAAAGGCUCACGCUGACAA | 211 | UUGUCAGCGUGAGCCUUUAGGAC | 339 |
| 154L5 | CUUAAAGGCUCACGCUGACAA | 200 | UUGUCAGCGUGAGCCUUUAAGUG | 340 |
| 155M5 | CUUAAAGGCUCACGCUGACAA | 200 | UUGUCAGCGUGAGCCUUUAAGUU | 341 |
| 156N5 | CGGAAAGGCUCACGCUGACAA | 212 | UUGUCAGCGUGAGCCUUUCGGAC | 342 |
| 157O5 | UUAAAGGCUCACGCUGACAAA | 213 | UUUGUCAGCGUGAGCCUUUAAGA | 343 |
| 158P5 | AGGCUCACGCUGACAAGCAGA | 214 | UCUGCUUGUCAGCGUGAGCCUUU | 344 |
| 159Q5 | UGCAGCGGCAGAGGCGGGAGA | 215 | UCUCCCGCCUCUGCCGCUGCACG | 345 |
| 160R5 | GCGGCAGAGGCGGGAGAUGGU | 216 | ACCAUCUCCCGCCUCUGCCGCUG | 346 |
| 161S5 | GAGGCGGGAGAUGGUGGCACA | 217 | UGUGCCACCAUCUCCCGCCUCUG | 347 |
| 162T5 | GCGGGAGAUGGUGGCACAGCA | 218 | UGCUGUGCCACCAUCUCCCGCCU | 348 |
| 163U5 | GUGGCACAGCAGCAUCGGCUA | 219 | UAGCCGAUGCUGCUGUGCCACCA | 349 |
| 164V5 | CAGCAGCAUCGGCUGCGACAA | 220 | UUGUCGCAGCCGAUGCUGCUGUG | 350 |
| 165W5 | AGCAGCAUCGGCUGCGACAGA | 221 | UCUGUCGCAGCCGAUGCUGCUGU | 351 |
| 166X5 | GCAGCAUCGGCUGCGACAGAA | 222 | UUCUGUCGCAGCCGAUGCUGCUG | 352 |
| 167Y5 | CAGCAUCGGCUGCGACAGAUA | 223 | UAUCUGUCGCAGCCGAUGCUGCU | 353 |
| 168Z5 | AGCAUCGGCUGCGACAGAUCA | 224 | UGAUCUGUCGCAGCCGAUGCUGC | 354 |
| 169a6 | GCAUCGGCUGCGACAGAUCCA | 225 | UGGAUCUGUCGCAGCCGAUGCUG | 355 |
| 170b6 | CAUCGGCUGCGACAGAUCCAA | 226 | UUGGAUCUGUCGCAGCCGAUGCU | 356 |
| 171c6 | UCGGCUGCGACAGAUCCAGGA | 227 | UCCUGGAUCUGUCGCAGCCGAUG | 357 |
| 172d6 | GGCUGCGACAGAUCCAGGAGA | 228 | UCUCCUGGAUCUGUCGCAGCCGA | 358 |
| 173e6 | GCUGCGACAGAUCCAGGAGAA | 229 | UUCUCCUGGAUCUGUCGCAGCCG | 359 |
| 174e7 | CUGCGACAGAUCCAGGAGAGA | 230 | UCUCUCCUGGAUCUGUCGCAGCC | 360 |

### EXAMPLE 6

To quantify levels of the Formula 1 conjugated siRNA strands in Table 13 in tissue samples, the tissue samples are homogenized in Clarity OTX cell lysis buffer (Phenomenex) to a final tissue concentration of 100 mg/ml. (For the sense strands, the OH metabolite is quantified because of rapid dephosphorylation in vivo). Plasma samples are diluted in Clarity OTX buffer 1:10 (v/v). Samples are subjected to solid phase extraction using a weak ion exchange resin (Waters, Oasis μElution SPE plate). Samples are eluted and subjected to liquid chromatography-high resolution mass spectrometry (LC-HRMS) as described in ASSAY and Drug Development Technologies, 10(3) pages 278-288 (2012)

Plasma exposure in mouse or cynomolgus monkeys is measured following subcutaneous administration through 6 hrs in and 24 hrs in monkey. Liver exposure is determined in mice at 6, 24, 72, 168, 336 and 1343 hrs. Results are subjected to non-compartmental analysis using the Phoenix software NCA package. Cₘₐₓ, t_{1/2} and AUC is determined in plasma for both species, liver Cₘₐₓ, t_{1/2} and AUC is determined in mouse, and liver t_{1/2} and AUC is determined in monkey.

Table 13 shows the liver exposure of 6 conjugated siRNAs in cynomolgus monkeys. Livers were harvested and subject to the above detection method using LC/MS about 2 and 12 weeks after treatment with subcutaneous 3 mg/kg of the listed ANGPTL8 siRNAs conjugated (at the 3′ end nucleotide of the sense strand) to the GalNac containing moiety of Formula I via Linker 2 (having Formula III).

Tables 12a and 12b show two exemplary experiments of the percent knockdown of ANGPTL8 mRNA as determined by RT-PCR of liver homogenate that is harvested from cynomolgus monkeys pre (1 monkey) and post dose (several monkeys as noted below, each 3 mg/kg) of conjugated ANGPTL8 siRNAs, where the GalNac containing moiety of Formula I is conjugated to the 3′ end nucleotide of the sense strand, via Linker 2 (having Formula III).

TABLE 12a
Cynomolgus monkey
Potency and Durability

| | | | 15 Days | 57 Days | 85 Days |
|---|---|---|---|---|---|
| | | | ANGPTL8 | ANGPTL8 | ANGPTL8 |
| | | | mRNA | mRNA | mRNA |
| | | | (% KD ± SE | (% KD from | (% KD from |
| | Sense | Antisense | from predose | predose | predose |
| siRNA | SEQ ID | SEQ ID | biopsy) | biopsy) | biopsy) |
| conjugate | NO | NO | 3 mg/kg | 3 mg/kg | 3 mg/kg |
| iii# | 363 | 369 | 34 | 40 | 5 |
| iv# | 364 | 370 | 21 | 19 | 45 |
| v* | 365 | 371 | 89 | 79 | 57 |
| vi* | 366 | 372 | 69 | 63 | 12. |

#n = 4
*n = 5

TABLE 12b
Cynomolgus monkey
Potency and Durability

| | | | 15 Days | 57 Days | 85 Days |
|---|---|---|---|---|---|
| | | | ANGPTL8 | ANGPTL8 | ANGPTL8 |
| | | | mRNA | mRNA | mRNA |
| | Sense | Antisense | (% KD from | (% KD from | (% KD from |
| siRNA | SEQ ID | SEQ ID | predose biopsy) | predose biopsy) | predose biopsy) |
| conjugate | NO | NO | 3 mg/kg | 3 mg/kg | 3 mg/kg |
| i | 361 | 367 | 88 ± 5*** | 74 ± 10** | 63 ± 37* |
| ii | 362 | 368 | 69 ± 31 | 54 ± 18 | 25 ± 30 |

n = 6
*P ≤ 0.05,
**P ≤ 0.01,
***P ≤ 0.005 vs pre-dose biopsy is statistically significant by MMRM

TABLE 13
Cynomolgus
PK Properties

| | | | 2 week | 12 week | |
|---|---|---|---|---|---|
| | Sense | Antisense | Liver | Liver | Liver |
| siRNA | SEQ ID | SEQ ID | exposure | exposure | t_{1/2} |
| conjugate | NO | NO | (μg/g) | (μg/g) | (days) |
| i | 361 | 367 | 22.0 | 1.9 | 33 |
| ii | 362 | 368 | 4.6 | <0.1 | <9 |
| iii | 363 | 369 | 9.0 | 0.1 | 15 |
| iv | 364 | 370 | 5.9 | 0.3 | 29 |
| v | 365 | 371 | 11.0 | 0.8 | 25 |
| vi | 366 | 372 | 5.4 | 0.4 | 30 |

The following numbered paragraphs provide additional embodiments of the RNAi agents and RNAi molecules disclosed herein:
1. An RNA interference (RNAi) molecule or RNAi agent comprising Formula I:

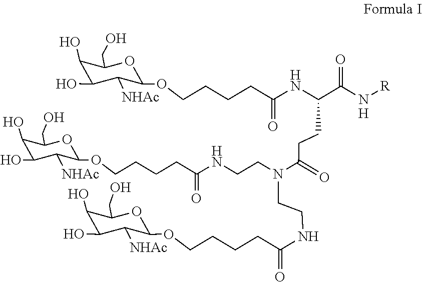

and a one or more oligonucleotides comprising 15 to 40 nucleotides that bind SEQ ID NO:1 and R is conjugated to an oligonucleotide, optionally via a linker.
2. The RNAi molecule or RNAi agent of paragraph 1, wherein the one or more oligonucleotides that bind one or more of the sequences as set forth in Table 1 or Table 2.
3. The RNAi molecule or RNAi agent of paragraph 1, wherein the one or more oligonucleotides comprise a sequence as set forth in Table 2.
4. The RNAi molecule or RNAi agent of any one of paragraphs 1 to 3, wherein the one or more oligonucleotides comprises one or more modified nucleotides.
5. The RNAi molecule or RNAi agent of paragraph 4, wherein the one or more modified nucleotides are modified on the 2′ position of the sugar, or on the pyrimidine or purine ring, or both.
6. The RNAi molecule or RNAi agent of paragraph 4 or 5, wherein the one or more modified nucleotides is a modified nucleotide comprising a modified nucleotide comprising a 2′ halogenated sugar group, a modified nucleotide comprising a 2′ methylated sugar group, a modified nucleotide comprising a 2′ methoxylated sugar group, a modified nucleotide comprising a methylated purine, or a modified nucleotide comprising a methylated pyrimidine ring, or any combination thereof.
7. The RNAi molecule or RNAi agent of any one of paragraphs 4 to 6, wherein the one or more modified nucleotides are modified on the 2′ position of the sugar and the modification comprises one or more 2′ fluoro groups or one or more 2′ methoxy groups, or both.
8. The RNAi molecule or RNAi agent of any one of paragraphs 4 to 7, wherein the one or more modified nucleotides are modified on the 2′ position of the sugar, and the sugar is ribose.
9. The RNAi molecule or RNAi agent of any one of paragraphs 1 to 8, wherein the one or more oligonucleotides comprise one or more modified bonds, and wherein the one or more modified bonds is a phosphorothioate bond.
10. The RNAi molecule or RNAi agent of any one of paragraphs 1 to 9, wherein the one or more oligonucleotides comprise an siRNA, and wherein the siRNA comprises a sense strand and an antisense strand.
11. The RNAi molecule or RNAi agent of paragraph 10, wherein the sense strand and the antisense strand are each independently between 15 to 40 nucleotides in length.
12. The RNAi molecule or RNAi agent of any one of paragraphs 1 to 11, further comprising a linker.
13. The RNAi molecule or RNAi agent of paragraph 12, wherein the linker comprises one of the following:

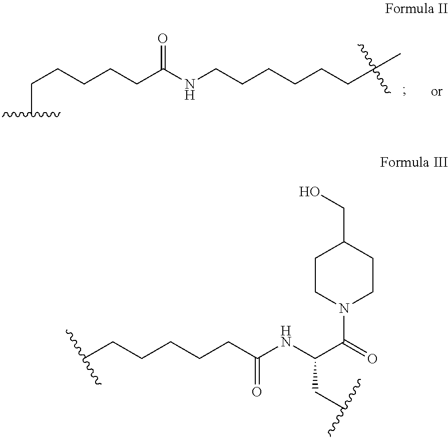 [SMILES: *CCCCCCNC(=O)CCCCC* ; *CCCCCC(=O)N[C@@H](C*)C(=O)N1CCC(CO)CC1]

14. The RNAi molecule or RNAi agent of any one of paragraphs 1 to 13, wherein the one or more oligonucleotides comprise an siRNA.
15. The RNAi molecule or RNAi agent of paragraph 14, wherein the siRNA comprises a sense strand and an antisense strand.
16. The RNAi molecule or RNAi agent of paragraph 15, wherein the sense strand and the antisense strand are each independently between 15 to 40 nucleotides in length.
17. The RNAi molecule or RNAi agent of any one of paragraphs 14 to 16, wherein the sense strand and the antisense strand are each independently between 18 to 25 nucleotides in length.
18. The RNAi molecule or RNAi agent of paragraph 17, wherein the sense strand and the antisense strand anneal, and optionally comprise one or more 5′ or 3′ nucleotide overhangs.
19. The RNAi molecule or RNAi agent of any one of paragraphs 14 to 18, wherein the 5′ end of the antisense strand is optionally phosphorylated.
20. The RNAi molecule or RNAi agent of any one of paragraphs 1 to 19, comprising a compound of Formula I and the one or more oligonucleotides that bind to any one of the sequences having SEQ ID NO:3 to SEQ ID NO:123 as shown in Table 1.
21. The RNAi molecule or RNAi agent of any one of paragraphs 1 to 20, comprising a compound of Formula I and one or more oligonucleotides that bind to any one of the sequences shown in Table 2.
22. The RNAi molecule or RNAi agent of any of the preceding paragraphs, wherein one or more nucleotides are modified on the 2′ position of the ribose.
23. The RNAi molecule or RNAi agent of any of the preceding paragraphs, wherein the ribose of at least one nucleotide is modified with a 2′ fluoro group or a 2′ methoxy group.
24. The RNAi molecule or RNAi agent of any of the preceding paragraphs, wherein the siRNA comprises one or more modified bonds.
25. The RNAi molecule or RNAi agent of paragraph 24, wherein the one or more modified bonds is a phosphorothioate bond.
26. The RNAi molecule or RNAi agent of any one of paragraphs 1 to 25, wherein the siRNA comprises a sense strand comprising a sequence of any one of SEQ ID NO:361, SEQ ID NO:362, SEQ ID NO:363, SEQ ID NO:364, SEQ ID NO:365, or SEQ ID NO:366.
27. The RNAi molecule or RNAi agent of any one of paragraphs 1 to 25, wherein the siRNA comprises an antisense strand comprising a sequence of any one of SEQ ID NO:367, SEQ ID NO:368, SEQ ID NO:369, SEQ ID NO:370, SEQ ID NO:371, or SEQ ID NO:372.
28. The RNAi molecule or RNAi agent of any one of paragraphs 1 to 27, wherein the siRNA comprises a sense strand and an antisense strand selected from the pairs of sequences as set forth in a-f:
   a. SEQ ID NO: 361 and SEQ ID NO:367;
   b. SEQ ID NO:362 and SEQ ID NO:368;
   c. SEQ ID NO: 363 and SEQ ID NO:369;
   d. SEQ ID NO: 364 and SEQ ID NO:370;
   e. SEQ ID NO: 365 and SEQ ID NO:371; or
   f. SEQ ID NO: 366 and SEQ ID NO:372.
29. A pharmaceutical composition comprising an RNAi molecule or RNAi agent of any one of paragraphs 1 to 28 and at least one pharmaceutically acceptable excipient.
30. A method of treating dyslipidemia comprising administering an RNAi molecule or RNAi agent of any one of paragraphs 1 to 28, or a pharmaceutical composition thereof, to a patient in need thereof.
31. A method of treating a cardiovascular disease comprising administering an effective amount of the RNAi molecule or RNAi agent of any one of paragraphs 1 to 28, or a pharmaceutical composition thereof, to a patient in need thereof
32. A method of preventing a cardiovascular event comprising administering an effective amount of an RNAi molecule or RNAi agent of any one of paragraphs 1 to 28, or a pharmaceutical composition thereof, to a patient in need thereof.
33. The method of paragraph 32, wherein the cardiovascular event is myocardial infarction.
34. A method of decreasing hospitalizations related to cardiovascular disease or events comprising administering an RNAi molecule or RNAi agent of any one of paragraphs 1 to 28, or a pharmaceutical composition thereof, to a patient in need thereof
35. A method of treating non-alcoholic fatty liver disease (NAFLD) comprising administering an RNAi molecule or RNAi agent of any one of paragraphs 1 to 28, or a pharmaceutical composition thereof, to a patient in need thereof.
36. The method of paragraph 35, wherein the NAFLD is non-alcoholic steatohepatitis (NASH).
37. A method of lowering triglyceride levels, comprising administering an RNAi molecule or RNAi agent of any one of paragraphs 1 to 28, or a pharmaceutical composition thereof, to a patient in need thereof
38. A method of decreasing inhibition of lipoprotein lipase (LPL) comprising administering an RNAi molecule or RNAi agent of any one of paragraphs 1 to 28, or a pharmaceutical composition thereof, to a patient in need thereof.
39. A method of increasing catabolism of triglyceride rich lipoproteins comprising administering an RNAi molecule or RNAi agent of any one of paragraphs 1 to 28, or a pharmaceutical composition thereof, to a patient in need thereof.
40. A method of treating a liver disease in a patient that would benefit from decreasing expression levels of ANGPTL8, comprising administering an RNAi molecule or RNAi agent of any one of paragraphs 1 to 28, or a pharmaceutical composition thereof, to a patient in need thereof.

SEQUENCE LISTING
Homo sapiens angiopoietin like 8 (ANGPTL8)
NCBI Reference Sequence: NM_018687.7
SEQ ID NO: 1
ATACCTTAGA CCCTCAGTCA TGCCAGTGCC TGCTCTGTGC
CTGCTCTGGG CCCTGGCAAT GGTGACCCGG CCTGCCTCAG
CGGCCCCCAT GGGCGGCCCA GAACTGGCAC AGCATGAGGA
GCTGACCCTG CTCTTCCATG GGACCCTGCA GCTGGGCCAG
GCCCTCAACG GTGTGTACAG GACCACGGAG GGACGGCTGA
CAAAGGCCAG GAACAGCCTG GGTCTCTATG GCCGCACAAT
AGAACTCCTG GGGCAGGAGG TCAGCCGGGG CCGGGATGCA
GCCCAGGAAC TTCGGGCAAG CCTGTTGGAG ACTCAGATGG
AGGAGGATAT TCTGCAGCTG CAGGCAGAGG CCACAGCTGA
GGTGCTGGGG GAGGTGGCCC AGGCACAGAA GGTGCTACGG
GACAGCGTGC AGCGGCTAGA AGTCCAGCTG AGGAGCGCCT
GGCTGGGCCC TGCCTACCGA GAATTTGAGG TCTTAAAGGC
TCACGCTGAC AAGCAGAGCC ACATCCTATG GGCCCTCACA
GGCCACGTGC AGCGGCAGAG GCGGGAGATG GTGGCACAGC
AGCATCGGCT GCGACAGATC CAGGAGAGAC TCCACACAGC
GGCGCTCCCA GCCTGAATCT GCCTGGATGG AACTGAGGAC
CAATCATGCT GCAAGGAACA CTTCCACGCC CCGTGAGGCC
CCTGTGCAGG GAGGAGCTGC CTGTTCACTG GGATCAGCCA
GGGCGCCGGG CCCCACTTCT GAGCACAGAG CAGAGACAGA
CGCAGGCGGG GACAAAGGCA GAGGATGTAG CCCCATTGGG
GAGGGGTGGA GGAAGGACAT GTACCCTTTC ATGCCTACAC
ACCCCTCATT AAAGCAGAGT CGTGGCATCT GA
ANGPTL3 sequence used for expression in mice
SEQ ID NO: 2
ATGTTCACCATCAAGCTGCTGCTGTTCATCGTGCCCCTCGTGATCAGCA
GCAGAATCGACCAGGACAACAGCAGCTTCGACAGCCTGAGCCCCGAGCC
CAAGAGCAGATTCGCCATGCTGGACGACGTGAAGATCCTGGCCAACGGC
CTGCTGCAGCTGGGCCACGGCCTGAAGGATTTCGTGCACAAGACCAAGG
GCCAGATCAACGACATCTTCCAGAAGCTGAACATCTTCGACCAGAGCTT
CTACGACCTGAGCCTGCAGACCAGCGAGATCAAAGAGGAAGAGAAAGAG
CTGCGGAGGACCACCTACAAGCTGCAAGTGAAGAACGAGGAAGTGAAAA
ACATGAGCCTGGAACTGAACAGCAAGCTGGAAAGCCTGCTGGAAGAAAA
GATTCTGCTGCAGCAGAAAGTGAAGTACCTGGAAGAACAGCTGACCAAC
CTGATCCAGAACCAGCCCGAGACACCCGAGCACCCCGAAGTGACCAGCC
TGAAAACCTTCGTGGAAAAGCAGGACAACTCCATCAAGGACCTGCTGCA
GACCGTGGAAGATCAGTACAAGCAGCTGAACCAGCAGCACTCCCAGATC
AAAGAAATCGAGAACCAGCTGAGGCGGACCAGCATCCAGGAACCCACCG
AGATCAGCCTGTCCAGCAAGCCCAGAGCCCCCAGAACAACCCCATTCCT
GCAGCTGAATGAGATCCGGAACGTGAAGCACGACGGCATCCCTGCCGAG
TGCACCACCATCTACAACAGAGGCGAGCACACCAGCGGGATGTACGCCA
TCAGACCCAGCAACAGCCAGGTGTTCCACGTGTACTGCGACGTGATCAG
CGGCAGCCCCTGGACACTGATCCAGCACAGAATCGATGGCAGCCAGAAC
TTCAACGAGACATGGGAGAACTATAAGTACGGCTTCGGCAGACTGGACG
GCGAGTTTTGGCTGGGCCTGGAAAAGATCTACAGCATCGTGAAGCAGAG
CAACTACGTGCTGAGAATCGAGCTGGAAGATTGGAAGGACAACAAGCAC
TACATCGAGTACAGCTTCTACCTGGGCAACCACGAGACAAACTACACCC
TGCACCTGGTGGCCATCACCGGCAACGTGCCAAACGCCATCCCCGAGAA
CAAGGATCTGGTGTTCAGCACCTGGGACCACAAGGCTAAGGGCCACTTC
AACTGCCCCGAGGGCTACTCTGGCGGCTGGTGGTGGCATGATGAGTGCG
GCGAGAACAACCTGAACGGCAAGTACAACAAGCCCAGGGCCAAGAGCAA
GCCTGAGAGAAGAAGAGGCCTGTCCTGGAAGTCCCAGAACGGCAGGCTG
TACTCTATCAAGAGCACCAAGATGCTGATCCACCCCACCGACAGCGAGA
GCTTCGAGTGATAA

TABLE 1
Target and antisense sequences for designed siRNAs

| SEQ | | | | SEQ | |
|---|---|---|---|---|---|
| ID NO | Start | End | 18 mer Target DNA | ID NO | 18 mer Antisense |
| (DNA | Position | Position | Sequence | (RNA | RNA sequence |
| SEQ) | on DNA | on DNA | (5′-3′) | SEQ) | (5′-3′) |
| 3 | 120 | 137 | AGCTGACCCTGCTCTTCC | 405 | GGAAGAGCAGGGUCAGCU |
| 4 | 141 | 158 | GGACCCTGCAGCTGGGCC | 406 | GGCCCAGCUGCAGGGUCC |
| 5 | 150 | 167 | AGCTGGGCCAGGCCCTCA | 407 | UGAGGGCCUGGCCCAGCU |
| 6 | 185 | 202 | ACGGAGGGACGGCTGACA | 408 | UGUCAGCCGUCCCUCCGU |
| 7 | 186 | 203 | CGGAGGGACGGCTGACAA | 409 | UUGUCAGCCGUCCCUCCG |
| 8 | 191 | 208 | GGACGGCTGACAAAGGCC | 410 | GGCCUUUGUCAGCCGUCC |
| 9 | 194 | 211 | CGGCTGACAAAGGCCAGG | 411 | CCUGGCCUUUGUCAGCCG |
| 10 | 195 | 212 | GGCTGACAAAGGCCAGGA | 412 | UCCUGGCCUUUGUCAGCC |
| 11 | 197 | 214 | CTGACAAAGGCCAGGAAC | 413 | GUUCCUGGCCUUUGUCAG |
| 12 | 210 | 227 | GGAACAGCCTGGGTCTCT | 414 | AGAGACCCAGGCUGUUCC |
| 13 | 211 | 228 | GAACAGCCTGGGTCTCTA | 415 | UAGAGACCCAGGCUGUUC |
| 14 | 215 | 232 | AGCCTGGGTCTCTATGGC | 416 | GCCAUAGAGACCCAGGCU |
| 15 | 217 | 234 | CCTGGGTCTCTATGGCCG | 417 | CGGCCAUAGAGACCCAGG |
| 16 | 218 | 235 | CTGGGTCTCTATGGCCGC | 418 | GCGGCCAUAGAGACCCAG |
| 17 | 219 | 236 | TGGGTCTCTATGGCCGCA | 419 | UGCGGCCAUAGAGACCCA |
| 18 | 220 | 237 | GGGTCTCTATGGCCGCAC | 420 | GUGCGGCCAUAGAGACCC |
| 19 | 259 | 276 | GGTCAGCCGGGGCCGGGA | 421 | UCCCGGCCCCGGCUGACC |
| 20 | 267 | 284 | GGGGCCGGGATGCAGCCC | 422 | GGGCUGCAUCCCGGCCCC |
| 21 | 270 | 287 | GCCGGGATGCAGCCCAGG | 423 | CCUGGGCUGCAUCCCGGC |
| 22 | 271 | 288 | CCGGGATGCAGCCCAGGA | 424 | UCCUGGGCUGCAUCCCGG |
| 23 | 273 | 290 | GGGATGCAGCCCAGGAAC | 425 | GUUCCUGGGCUGCAUCCC |
| 24 | 274 | 291 | GGATGCAGCCCAGGAACT | 426 | AGUUCCUGGGCUGCAUCC |
| 25 | 281 | 298 | GCCCAGGAACTTCGGGCA | 427 | UGCCCGAAGUUCCUGGGC |
| 26 | 282 | 299 | CCCAGGAACTTCGGGCAA | 428 | UUGCCCGAAGUUCCUGGG |
| 27 | 283 | 300 | CCAGGAACTTCGGGCAAG | 429 | CUUGCCCGAAGUUCCUGG |
| 28 | 284 | 301 | CAGGAACTTCGGGCAAGC | 430 | GCUUGCCCGAAGUUCCUG |
| 29 | 285 | 302 | AGGAACTTCGGGCAAGCC | 431 | GGCUUGCCCGAAGUUCCU |
| 30 | 286 | 303 | GGAACTTCGGGCAAGCCT | 432 | AGGCUUGCCCGAAGUUCC |
| 31 | 287 | 304 | GAACTTCGGGCAAGCCTG | 433 | CAGGCUUGCCCGAAGUUC |
| 32 | 290 | 307 | CTTCGGGCAAGCCTGTTG | 434 | CAACAGGCUUGCCCGAAG |
| 33 | 291 | 308 | TTCGGGCAAGCCTGTTGG | 435 | CCAACAGGCUUGCCCGAA |
| 34 | 293 | 310 | CGGGCAAGCCTGTTGGAG | 436 | CUCCAACAGGCUUGCCCG |
| 35 | 297 | 314 | CAAGCCTGTTGGAGACTC | 437 | GAGUCUCCAACAGGCUUG |
| 36 | 299 | 316 | AGCCTGTTGGAGACTCAG | 438 | CUGAGUCUCCAACAGGCU |
| 37 | 300 | 317 | GCCTGTTGGAGACTCAGA | 439 | UCUGAGUCUCCAACAGGC |
| 38 | 303 | 320 | TGTTGGAGACTCAGATGG | 440 | CCAUCUGAGUCUCCAACA |
| 39 | 304 | 321 | GTTGGAGACTCAGATGGA | 441 | UCCAUCUGAGUCUCCAAC |
| 40 | 309 | 326 | AGACTCAGATGGAGGAGG | 442 | CCUCCUCCAUCUGAGUCU |
| 41 | 310 | 327 | GACTCAGATGGAGGAGGA | 443 | UCCUCCUCCAUCUGAGUC |
| 42 | 311 | 328 | ACTCAGATGGAGGAGGAT | 444 | AUCCUCCUCCAUCUGAGU |
| 43 | 312 | 329 | CTCAGATGGAGGAGGATA | 445 | UAUCCUCCUCCAUCUGAG |
| 44 | 313 | 330 | TCAGATGGAGGAGGATAT | 446 | AUAUCCUCCUCCAUCUGA |
| 45 | 314 | 331 | CAGATGGAGGAGGATATT | 447 | AAUAUCCUCCUCCAUCUG |
| 46 | 315 | 332 | AGATGGAGGAGGATATTC | 448 | GAAUAUCCUCCUCCAUCU |
| 47 | 316 | 333 | GATGGAGGAGGATATTCT | 449 | AGAAUAUCCUCCUCCAUC |
| 48 | 317 | 334 | ATGGAGGAGGATATTCTG | 450 | CAGAAUAUCCUCCUCCAU |
| 49 | 319 | 336 | GGAGGAGGATATTCTGCA | 451 | UGCAGAAUAUCCUCCUCC |
| 50 | 321 | 338 | AGGAGGATATTCTGCAGC | 452 | GCUGCAGAAUAUCCUCCU |
| 51 | 372 | 389 | AGGTGGCCCAGGCACAGA | 453 | UCUGUGCCUGGGCCACCU |
| 52 | 379 | 396 | CCAGGCACAGAAGGTGCT | 454 | AGCACCUUCUGUGCCUGG |
| 53 | 411 | 428 | AGCGGCTAGAAGTCCAGC | 455 | GCUGGACUUCUAGCCGCU |
| 54 | 412 | 429 | GCGGCTAGAAGTCCAGCT | 456 | AGCUGGACUUCUAGCCGC |
| 55 | 413 | 430 | CGGCTAGAAGTCCAGCTG | 457 | CAGCUGGACUUCUAGCCG |
| 56 | 414 | 431 | GGCTAGAAGTCCAGCTGA | 458 | UCAGCUGGACUUCUAGCC |
| 57 | 415 | 432 | GCTAGAAGTCCAGCTGAG | 459 | CUCAGCUGGACUUCUAGC |
| 58 | 420 | 437 | AAGTCCAGCTGAGGAGCG | 460 | CGCUCCUCAGCUGGACUU |
| 59 | 421 | 438 | AGTCCAGCTGAGGAGCGC | 461 | GCGCUCCUCAGCUGGACU |
| 60 | 438 | 455 | CCTGGCTGGGCCCTGCCT | 462 | AGGCAGGGCCCAGCCAGG |
| 61 | 459 | 476 | GAGAATTTGAGGTCTTAA | 463 | UUAAGACCUCAAAUUCUC |
| 62 | 460 | 477 | AGAATTTGAGGTCTTAAA | 464 | UUUAAGACCUCAAAUUCU |
| 63 | 462 | 479 | AATTTGAGGTCTTAAAGG | 465 | CCUUUAAGACCUCAAAUU |
| 64 | 463 | 480 | ATTTGAGGTCTTAAAGGC | 466 | GCCUUUAAGACCUCAAAU |
| 65 | 464 | 481 | TTTGAGGTCTTAAAGGCT | 467 | AGCCUUUAAGACCUCAAA |
| 66 | 465 | 482 | TTGAGGTCTTAAAGGCTC | 468 | GAGCCUUUAAGACCUCAA |
| 67 | 466 | 483 | TGAGGTCTTAAAGGCTCA | 469 | UGAGCCUUUAAGACCUCA |
| 68 | 467 | 484 | GAGGTCTTAAAGGCTCAC | 470 | GUGAGCCUUUAAGACCUC |
| 69 | 469 | 486 | GGTCTTAAAGGCTCACGC | 471 | GCGUGAGCCUUUAAGACC |
| 70 | 471 | 488 | TCTTAAAGGCTCACGCTG | 472 | CAGCGUGAGCCUUUAAGA |
| 71 | 472 | 489 | CTTAAAGGCTCACGCTGA | 473 | UCAGCGUGAGCCUUUAAG |
| 72 | 473 | 490 | TTAAAGGCTCACGCTGAC | 474 | GUCAGCGUGAGCCUUUAA |
| 73 | 474 | 491 | TAAAGGCTCACGCTGACA | 475 | UGUCAGCGUGAGCCUUUA |
| 74 | 475 | 492 | AAAGGCTCACGCTGACAA | 476 | UUGUCAGCGUGAGCCUUU |
| 75 | 476 | 493 | AAGGCTCACGCTGACAAG | 477 | CUUGUCAGCGUGAGCCUU |
| 76 | 477 | 494 | AGGCTCACGCTGACAAGC | 478 | GCUUGUCAGCGUGAGCCU |
| 77 | 479 | 496 | GCTCACGCTGACAAGCAG | 479 | CUGCUUGUCAGCGUGAGC |
| 78 | 480 | 497 | CTCACGCTGACAAGCAGA | 480 | UCUGCUUGUCAGCGUGAG |
| 79 | 481 | 498 | TCACGCTGACAAGCAGAG | 481 | CUCUGCUUGUCAGCGUGA |
| 80 | 482 | 499 | CACGCTGACAAGCAGAGC | 482 | GCUCUGCUUGUCAGCGUG |
| 81 | 483 | 500 | ACGCTGACAAGCAGAGCC | 483 | GGCUCUGCUUGUCAGCGU |
| 82 | 485 | 502 | GCTGACAAGCAGAGCCAC | 484 | GUGGCUCUGCUUGUCAGC |
| 83 | 513 | 530 | CCCTCACAGGCCACGTGC | 485 | GCACGUGGCCUGUGAGGG |
| 84 | 514 | 531 | CCTCACAGGCCACGTGCA | 486 | UGCACGUGGCCUGUGAGG |
| 85 | 520 | 537 | AGGCCACGTGCAGCGGCA | 487 | UGCCGCUGCACGUGGCCU |
| 86 | 521 | 538 | GGCCACGTGCAGCGGCAG | 488 | CUGCCGCUGCACGUGGCC |
| 87 | 524 | 541 | CACGTGCAGCGGCAGAGG | 489 | CCUCUGCCGCUGCACGUG |
| 88 | 530 | 547 | CAGCGGCAGAGGCGGGAG | 490 | CUCCCGCCUCUGCCGCUG |
| 89 | 534 | 551 | GGCAGAGGCGGGAGATGG | 491 | CCAUCUCCCGCCUCUGCC |
| 90 | 540 | 557 | GGCGGGAGATGGTGGCAC | 492 | GUGCCACCAUCUCCCGCC |
| 91 | 543 | 560 | GGGAGATGGTGGCACAGC | 493 | GCUGUGCCACCAUCUCCC |
| 92 | 546 | 563 | AGATGGTGGCACAGCAGC | 494 | GCUGCUGUGCCACCAUCU |
| 93 | 553 | 570 | GGCACAGCAGCATCGGCT | 495 | AGCCGAUGCUGCUGUGCC |
| 94 | 555 | 572 | CACAGCAGCATCGGCTGC | 496 | GCAGCCGAUGCUGCUGUG |
| 95 | 557 | 574 | CAGCAGCATCGGCTGCGA | 497 | UCGCAGCCGAUGCUGCUG |
| 96 | 558 | 575 | AGCAGCATCGGCTGCGAC | 498 | GUCGCAGCCGAUGCUGCU |
| 97 | 559 | 576 | GCAGCATCGGCTGCGACA | 499 | UGUCGCAGCCGAUGCUGC |
| 98 | 560 | 577 | CAGCATCGGCTGCGACAG | 500 | CUGUCGCAGCCGAUGCUG |
| 99 | 561 | 578 | AGCATCGGCTGCGACAGA | 501 | UCUGUCGCAGCCGAUGCU |
| 100 | 562 | 579 | GCATCGGCTGCGACAGAT | 502 | AUCUGUCGCAGCCGAUGC |
| 101 | 563 | 580 | CATCGGCTGCGACAGATC | 503 | GAUCUGUCGCAGCCGAUG |
| 102 | 564 | 581 | ATCGGCTGCGACAGATCC | 504 | GGAUCUGUCGCAGCCGAU |
| 103 | 565 | 582 | TCGGCTGCGACAGATCCA | 505 | UGGAUCUGUCGCAGCCGA |
| 104 | 567 | 584 | GGCTGCGACAGATCCAGG | 506 | CCUGGAUCUGUCGCAGCC |
| 105 | 569 | 586 | CTGCGACAGATCCAGGAG | 507 | CUCCUGGAUCUGUCGCAG |
| 106 | 570 | 587 | TGCGACAGATCCAGGAGA | 508 | UCUCCUGGAUCUGUCGCA |
| 107 | 571 | 588 | GCGACAGATCCAGGAGAG | 509 | CUCUCCUGGAUCUGUCGC |
| 108 | 607 | 624 | CCCAGCCTGAATCTGCCT | 510 | AGGCAGAUUCAGGCUGGG |
| 109 | 610 | 627 | AGCCTGAATCTGCCTGGA | 511 | UCCAGGCAGAUUCAGGCU |
| 110 | 611 | 628 | GCCTGAATCTGCCTGGAT | 512 | AUCCAGGCAGAUUCAGGC |
| 111 | 619 | 636 | CTGCCTGGATGGAACTGA | 513 | UCAGUUCCAUCCAGGCAG |
| 112 | 644 | 661 | TCATGCTGCAAGGAACAC | 514 | GUGUUCCUUGCAGCAUGA |
| 113 | 652 | 669 | CAAGGAACACTTCCACGC | 515 | GCGUGGAAGUGUUCCUUG |
| 114 | 655 | 672 | GGAACACTTCCACGCCCC | 516 | GGGGCGUGGAAGUGUUCC |
| 115 | 698 | 715 | TGCCTGTTCACTGGGATC | 517 | GAUCCCAGUGAACAGGCA |
| 116 | 699 | 716 | GCCTGTTCACTGGGATCA | 518 | UGAUCCCAGUGAACAGGC |
| 117 | 701 | 718 | CTGTTCACTGGGATCAGC | 519 | GCUGAUCCCAGUGAACAG |
| 118 | 702 | 719 | TGTTCACTGGGATCAGCC | 520 | GGCUGAUCCCAGUGAACA |
| 119 | 709 | 726 | TGGGATCAGCCAGGGCGC | 521 | GCGCCCUGGCUGAUCCCA |
| 120 | 710 | 727 | GGGATCAGCCAGGGCGCC | 522 | GGCGCCCUGGCUGAUCCC |
| 121 | 847 | 864 | CATTAAAGCAGAGTCGTG | 523 | CACGACUCUGCUUUAAUG |
| 122 | 848 | 865 | ATTAAAGCAGAGTCGTGG | 524 | CCACGACUCUGCUUUAAU |
| 123 | 850 | 867 | TAAAGCAGAGTCGTGGCA | 525 | UGCCACGACUCUGCUUUA |

TABLE 2

| SEQ | | | | SEQ | Antisense |
|---|---|---|---|---|---|
| ID NO | Start | End | 18 mer DNA | ID NO | RNA |
| (DNA | Position | Position | Sequence | (RNA | sequence |
| SEQ) | on DNA | on DNA | (5′-3′) | SEQ) | (5′-3′) |
| 3 | 120 | 137 | AGCTGACCCTGCTCTTCC | 405 | GGAAGAGCAGGGUCAGCU |
| 6 | 185 | 202 | ACGGAGGGACGGCTGACA | 408 | UGUCAGCCGUCCCUCCGU |
| 10 | 195 | 212 | GGCTGACAAAGGCCAGGA | 412 | UCCUGGCCUUUGUCAGCC |
| 11 | 197 | 214 | CTGACAAAGGCCAGGAAC | 413 | GUUCCUGGCCUUUGUCAG |
| 12 | 210 | 227 | GGAACAGCCTGGGTCTCT | 414 | AGAGACCCAGGCUGUUCC |
| 13 | 211 | 228 | GAACAGCCTGGGTCTCTA | 415 | UAGAGACCCAGGCUGUUC |
| 16 | 218 | 235 | CTGGGTCTCTATGGCCGC | 418 | GCGGCCAUAGAGACCCAG |
| 18 | 220 | 237 | GGGTCTCTATGGCCGCAC | 420 | GUGCGGCCAUAGAGACCC |
| 23 | 273 | 290 | GGGATGCAGCCCAGGAAC | 425 | GUUCCUGGGCUGCAUCCC |
| 24 | 274 | 291 | GGATGCAGCCCAGGAACT | 426 | AGUUCCUGGGCUGCAUCC |
| 26 | 282 | 299 | CCCAGGAACTTCGGGCAA | 428 | UUGCCCGAAGUUCCUGGG |
| 27 | 283 | 300 | CCAGGAACTTCGGGCAAG | 429 | CUUGCCCGAAGUUCCUGG |
| 30 | 286 | 303 | GGAACTTCGGGCAAGCCT | 432 | AGGCUUGCCCGAAGUUCC |
| 31 | 287 | 304 | GAACTTCGGGCAAGCCTG | 433 | CAGGCUUGCCCGAAGUUC |
| 32 | 290 | 307 | CTTCGGGCAAGCCTGTTG | 434 | CAACAGGCUUGCCCGAAG |
| 33 | 291 | 308 | TTCGGGCAAGCCTGTTGG | 435 | CCAACAGGCUUGCCCGAA |
| 34 | 293 | 310 | CGGGCAAGCCTGTTGGAG | 436 | CUCCAACAGGCUUGCCCG |
| 35 | 297 | 314 | CAAGCCTGTTGGAGACTC | 437 | GAGUCUCCAACAGGCUUG |
| 36 | 299 | 316 | AGCCTGTTGGAGACTCAG | 438 | CUGAGUCUCCAACAGGCU |
| 37 | 300 | 317 | GCCTGTTGGAGACTCAGA | 439 | UCUGAGUCUCCAACAGGC |
| 38 | 303 | 320 | TGTTGGAGACTCAGATGG | 440 | CCAUCUGAGUCUCCAACA |
| 39 | 304 | 321 | GTTGGAGACTCAGATGGA | 441 | UCCAUCUGAGUCUCCAAC |
| 40 | 309 | 326 | AGACTCAGATGGAGGAGG | 442 | CCUCCUCCAUCUGAGUCU |
| 41 | 310 | 327 | GACTCAGATGGAGGAGGA | 443 | UCCUCCUCCAUCUGAGUC |
| 42 | 311 | 328 | ACTCAGATGGAGGAGGAT | 444 | AUCCUCCUCCAUCUGAGU |
| 43 | 312 | 329 | CTCAGATGGAGGAGGATA | 445 | UAUCCUCCUCCAUCUGAG |
| 44 | 313 | 330 | TCAGATGGAGGAGGATAT | 446 | AUAUCCUCCUCCAUCUGA |
| 46 | 315 | 332 | AGATGGAGGAGGATATTC | 448 | GAAUAUCCUCCUCCAUCU |
| 47 | 316 | 333 | GATGGAGGAGGATATTCT | 449 | AGAAUAUCCUCCUCCAUC |
| 49 | 319 | 336 | GGAGGAGGATATTCTGCA | 451 | UGCAGAAUAUCCUCCUCC |
| 50 | 321 | 338 | AGGAGGATATTCTGCAGC | 452 | GCUGCAGAAUAUCCUCCU |
| 52 | 379 | 396 | CCAGGCACAGAAGGTGCT | 454 | AGCACCUUCUGUGCCUGG |
| 55 | 413 | 430 | CGGCTAGAAGTCCAGCTG | 457 | CAGCUGGACUUCUAGCCG |
| 56 | 414 | 431 | GGCTAGAAGTCCAGCTGA | 458 | UCAGCUGGACUUCUAGCC |
| 57 | 415 | 432 | GCTAGAAGTCCAGCTGAG | 459 | CUCAGCUGGACUUCUAGC |
| 61 | 459 | 476 | GAGAATTTGAGGTCTTAA | 463 | UUAAGACGUCAAAUUCUC |
| 62 | 460 | 477 | AGAATTTGAGGTCTTAAA | 464 | UUUAAGACGUCAAAUUCU |
| 63 | 462 | 479 | AATTTGAGGTCTTAAAGG | 465 | CCUUUAAGACCUCAAAUU |
| 64 | 463 | 480 | ATTTGAGGTCTTAAAGGC | 466 | GCCUUUAAGACCUCAAAU |
| 65 | 464 | 481 | TTTGAGGTCTTAAAGGCT | 467 | AGCCUUUAAGACCUCAAA |
| 66 | 465 | 482 | TTGAGGTCTTAAAGGCTC | 468 | GAGCCUUUAAGACCUCAA |
| 67 | 466 | 483 | TGAGGTCTTAAAGGCTCA | 469 | UGAGCCUUUAAGACCUCA |
| 69 | 469 | 486 | GGTCTTAAAGGCTCACGC | 471 | GCGUGAGCCUUUAAGACC |
| 70 | 471 | 488 | TCTTAAAGGCTCACGCTG | 472 | CAGCGUGAGCCUUUAAGA |
| 71 | 472 | 489 | CTTAAAGGCTCACGCTGA | 473 | UCAGCGUGAGCCUUUAAG |
| 72 | 473 | 490 | TTAAAGGCTCACGCTGAC | 474 | GUCAGCGUGAGCCUUUAA |
| 73 | 474 | 491 | TAAAGGCTCACGCTGACA | 475 | UGUCAGCGUGAGCCUUUA |
| 74 | 475 | 492 | AAAGGCTCACGCTGACAA | 476 | UUGUCAGCGUGAGCCUUU |
| 77 | 479 | 496 | GCTCACGCTGACAAGCAG | 479 | CUGCUUGUCAGCGUGAGC |
| 88 | 530 | 547 | CAGCGGCAGAGGCGGGAG | 490 | CUCCCGCCUCUGCCGCUG |
| 89 | 534 | 551 | GGCAGAGGCGGGAGATGG | 491 | CCAUCUCCCGCCUCUGCC |
| 90 | 540 | 557 | GGCGGGAGATGGTGGCAC | 492 | GUGCCACCAUCUCCCGCC |
| 91 | 543 | 560 | GGGAGATGGTGGCACAGC | 493 | GCUGUGCCACCAUCUCCC |
| 93 | 553 | 570 | GGCACAGCAGCATCGGCT | 495 | AGCCGAUGCUGCUGUGCC |
| 97 | 559 | 576 | GCAGCATCGGCTGCGACA | 499 | UGUCGCAGCCGAUGCUGC |
| 98 | 560 | 577 | CAGCATCGGCTGCGACAG | 500 | CUGUCGCAGCCGAUGCUG |
| 99 | 561 | 578 | AGCATCGGCTGCGACAGA | 501 | UCUGUCGCAGCCGAUGCU |
| 100 | 562 | 579 | GCATCGGCTGCGACAGAT | 502 | AUCUGUCGCAGCCGAUGC |
| 101 | 563 | 580 | CATCGGCTGCGACAGATC | 503 | GAUCUGUCGCAGCCGAUG |
| 102 | 564 | 581 | ATCGGCTGCGACAGATCC | 504 | GGAUCUGUCGCAGCCGAU |
| 103 | 565 | 582 | TCGGCTGCGACAGATCCA | 505 | UGGAUCUGUCGCAGCCGA |
| 104 | 567 | 584 | GGCTGCGACAGATCCAGG | 506 | CCUGGAUCUGUCGCAGCC |
| 105 | 569 | 586 | CTGCGACAGATCCAGGAG | 507 | CUCCUGGAUCUGUCGCAG |
| 106 | 570 | 587 | TGCGACAGATCCAGGAGA | 508 | UCUCCUGGAUCUGUCGCA |
| 107 | 571 | 588 | GCGACAGATCCAGGAGAG | 509 | CUCUCCUGGAUCUGUCGC |

TABLE 3

| | Sense | | |
|---|---|---|---|
| SEQ | or | | |
| ID | Anti- | Sense or Anti-Sense with | Delivery |
| NO | Sense | modifications (5′ to 3′) | Moiety |
| 381 | Sense | mC*mG*mAmGmAmAfUmUfUfGfA | Control |
| | | mGmGmUmCmUmUmAmA*mA*mG | GalNAc |
| 397 | Anti- | PmC*fU*mUmUmAfAmGmAmCmCm | |
| | sense | UmCmAfAmAfUmUmCmUmCmG* | |
| | | mG*mU | |
| 382 | Sense | mC*mG*mAmGmAmAfUmUfUfGfA | Formula |
| | | mGmGmUmCmUmUmAmA*mA*mG | I |
| 398 | Anti- | PmC*fU*mUmUmAfAmGmAmCmCm | |
| | sense | UmCmAfAmAfUmUmCmUmCmG* | |
| | | mG*mU | |
| 366 | Sense | mC*mG*mAmGmAmAfUmUfUfGfA | Formula |
| | | mGmGmUmCmUmUmAmA*mA*mG | I |
| 372 | Anti- | mC*fU*mUmUmAfAmGmAmCmCmU | |
| | sense | mCmAfAmAfUmUmCmUmCmG* | |
| | | mG*mU | |

TABLE 6

| | Sense | |
|---|---|---|
| SEQ | or | |
| ID | Anti- | Sense or Anti-Sense with |
| NO | Sense | modifications (5′ to 3′) |
| 373 | Sense | mU*mC*mAmGmAmUmGmGfAfGfGmAmGmGmAmUmAmU |
| | | mUmCmU |
| 389 | Anti- | PmA*fG*mAmAmUfAmUmCmCmUmCmCmUfCmCfAmU |
| | Sense | mCmUmGmA*mG*mU |
| 374 | Sense | mG*mC*mAmGmAmUfGmGfAfGfGmAmGmGmAmUmAmU |
| | | mU*mC*mA |
| 390 | Anti- | PmU*fG*mAmAmUfAmUmCmCmUmCmCmUfCmCfAmU |
| | Sense | mCmUmGmC*mG*mU |
| 375 | Sense | mU*mC*mAmGmAmUfGmGfAfGfGmAmGmGmAmUmAmU |
| | | mUmCmU |
| 391 | Anti- | PmA*fG*mAmAmUfAmUmCmCmUmCmCmUfCmCfAmU |
| | Sense | mCmUmGmA*mG*mU |
| 376 | Sense | mU*mC*mAmGmAmUfGmGfAfGfGmAmGmGmAmUmAmU |
| | | mU*mC*mC |
| 392 | Anti- | PmG*fG*mAmAmUfAmUmCmCmUmCmCmUfCmCfAmU |
| | Sense | mCmUmGmA*mG*mU |
| 377 | Sense | mU*mC*mAmGmAmUfGmGfAfGfGmAmGmGmAmUmAmU |
| | | mUmCmU |
| 393 | Anti- | PmA*fG*mAmAmUmAmUmCmCmUmCmCmUfCmCmAmU |
| | Sense | mCmUmGmA*mG*mU |
| 378 | Sense | mC*mA*mGmAmUmGfGmAfGfGfAmGmGmAmUmAmUmU |
| | | mC*mU*mA |
| 394 | Anti- | PmU*fA*mGmAmAfUmAmUmCmCmUmCmCfUmCfCmA |
| | Sense | mUmCmUmG*mA*mG |
| 379 | Sense | mA*mU*mGmGmAmGfGmAfGfGfAmUmAmUmUmCmUmG |
| | | mC*mA*mA |
| 395 | Anti- | PmU*fU*mGmCmAfGmAmAmUmAmUmCmCfUmCfCmU |
| | Sense | mCmCmAmU*mC*mU |
| 380 | Sense | mC*mC*mGmAmGmAfAmUfUfUfGmAmGmGmUmCmUmU |
| | | mA*mA*mA |
| 396 | Anti- | PmU*fU*mUmAmAfGmAmCmCmUmCmAmAfAmUfUmC |
| | Sense | mUmCmGmG*mU*mA |
| 381 | Sense | mC*mG*mAmGmAmAfUmUfUfGfAmGmGmUmCmUmUmA |
| | | mA*mA*mG |
| 397 | Anti- | PmC*fU*mUmUmAfAmGmAmCmCmUmCmAfAmAfUmU |
| | Sense | mCmUmCmG*mG*mU |
| 382 | Sense | mC*mG*mAmGmAmAfUmUfUfGfAmGmGmUmCmUmUmA |
| | | mA*mA*mG |
| 398 | Anti- | PmC*fU*mUmUmAfAmGmAmCmCmUmCmAfAmAfUmU |
| | Sense | mCmUmCmG*mG*mU |
| 383 | Sense | mG*mG*mAmGmAmAfUmUfUfGfAmGmGmUmCmUmUmA |
| | | mA*mA*mA |
| 399 | Anti- | PmU*fU*mUmUmAfAmGmAmCmCmUmCmAfAmAfUmU |
| | Sense | mCmUmCmC*mG*mU |
| 384 | Sense | mC*mG*mAmGmAmAfUmUfUfGfAmGmGmUmCmUmUmA |
| | | mA*mA*mA |
| 400 | Anti- | PmU*fU*mUmUmAfAmGmAmCmCmUmCmAfAmAfUmU |
| | Sense | mCmUmCmG*mG*mU |
| 385 | Sense | mC*mG*mAmGmAmAfUmUfUfGfAmGmGmUmCmUmUmA |
| | | mAmAmG |
| 401 | Anti- | PmC*fU*mUmUmAfAmGmAmCmCmUmCmAfAmAfUmU |
| | Sense | mCmUmCmG*mG*mU |
| 386 | Sense | mC*mG*mAmGmAmAmUmUfUfGfAmGmGmUmCmUmUmA |
| | | mAmAmG |
| 402 | Anti- | PmC*fU*mUmUmAfAmGmAmCmCmUmCmAfAmAfUmU |
| | Sense | mCmUmCmG*mG*mU |
| 387 | Sense | mC*mG*mAmGmAmAfUmUfUfGfAmGmGmUmCmUmUmA |
| | | mAmAmG |
| 403 | Anti- | PmC*fU*mUmUmAmAmGmAmCmCmUmCmAfAmAmUmU |
| | Sense | mCmUmCmG*mG*mU |
| 388 | Sense | mC*mG*mAmGmAmAmUmUfUfGfAmGmGmUmCmUmUmA |
| | | mAmAmG |
| 404 | Anti- | PmC*fU*mUmUmAmAmGmAmCmCmUmCmAfAmAmUmU |
| | Sense | mCmUmCmG*mG*mU |

TABLE 8

| | | Anti- | |
|---|---|---|---|
| Sense | Sense strand | Sense | Antisense |
| SEQ | sequence with | SEQ | strand with |
| ID | modifications | ID | modifications |
| NO | (5′ to 3′) | NO | (5′ to 3′) |
| 361 | mA*mU*mGmGmAmGf | 367 | [Phos]mU*fU*mGmCmA |
| | GmAfGfGfAmUmAmU | | fGmAmAmUmAmUmCmC |
| | mUmCmUmGmC*mA*mA | | fUmCfCmUmCmCmAmU* |
| | | | mC*mU |
| 362 | mC*mC*mGmAmGmAf | 368 | [Phos]mU*fU*mUmAmA |
| | AmUfUfUfGmAmGmG | | fGmAmCmCmUmCmAmA |
| | mUmCmUmUmA*mA*mA | | fAmUfUmCmUmCmGmG* |
| | | | mU*mA |
| 363 | mA*mU*mGmGmAmGf | 369 | mU*fU*mGmCmAfGmAmA |
| | GmAfGfGfAmUmAmU | | mUmAmUmCmCfUmCfC |
| | mUmCmUmGmC*mA*mA | | mUmCmCmAmU*mC*mU |
| 364 | mG*mG*mUmCmUmUf | 370 | mU*fC*mAmGmCfGmUmG |
| | AmAfAfGfGmCmUmC | | mAmGmCmCmUfUmUfA |
| | mAmCmGmCmU*mG*mA | | mAmGmAmCmC*mU*mC |
| 365 | mA*mU*mGmGmAmGf | 371 | mC*fU*mGmCmAfGmAmA |
| | GmAfGfGfAmUmAmU | | mUmAmUmCmCfUmCfC |
| | mUmCmUmGmC*mA*mG | | mUmCmCmAmU*mC*mU |
| 366 | mC*mG*mAmGmAmAf | 372 | mC*fU*mUmUmAfAmGmA |
| | UmUfUfGfAmGmGmU | | mCmCmUmCmAfAmAfU |
| | mCmUmUmAmA*mA*mG | | mUmCmUmCmG*mG*mU |

TABLE 11

| | | Sense | | Anti- |
|---|---|---|---|---|
| | | SEQ | | Sense |
| | | ID | | SEQ |
| Row | Sense Sequence | NO | Anti-Sense Sequence | ID NO |
| 1A | GGAGCUGACCCUGCUCUUCCA | 124 | UGGAAGAGCAGGGUCAGCUCCUC | 231 |
| 2B | CCACGGAGGGACGGCUGACAA | 125 | UUGUCAGCCGUCCCUCCGUGGUC | 232 |
| 3C | ACGGCUGACAAAGGCCAGGAA | 126 | UUCCUGGCCUUUGUCAGCCGUCC | 233 |
| 4D | GGCUGACAAAGGCCAGGAACA | 127 | UGUUCCUGGCCUUUGUCAGCCGU | 234 |
| 5E | CAGGAACAGCCUGGGUCUCUA | 128 | UAGAGACCCAGGCUGUUCCUGGC | 235 |
| 6F | CAGGAACAGCCUGGGUCUCUC | 129 | GAGAGACCCAGGCUGUUCCUGGC | 236 |
| 7G | AGGAACAGCCUGGGUCUCUAU | 130 | AUAGAGACCCAGGCUGUUCCUGG | 237 |
| 8H | AGGAACAGCCUGGGUCUCUAA | 131 | UUAGAGACCCAGGCUGUUCCUGG | 238 |
| 9I | GCCUGGGUCUCUAUGGCCGCA | 132 | UGCGGCCAUAGAGACCCAGGCUG | 239 |
| 10J | CUGGGUCUCUAUGGCCGCACA | 133 | UGUGCGGCCAUAGAGACCCAGGC | 240 |
| 11K | CUUGGUCUCUAUGGCCGCACA | 134 | UGUGCGGCCAUAGAGACCAAGGC | 241 |
| 12L | CUGGGUCUCUAUGGCCGCACA | 133 | UGUGCGGCCAUAGAGACCCAGUG | 242 |
| 13M | CUGGGUCUCUAUGGCCGCACA | 133 | UGUGCGGCCAUAGAGACCCAGGC | 243 |
| 14N | CUGGGUCUCUAUGGCCGCACA | 133 | UGUGCGGCCAUAGAGACCCAGUU | 244 |
| 15O | CUGGGUCUCUAUGGCCGCACA | 133 | UGUGCGGCCAUAGAGACCCAGAG | 245 |
| 16P | CUGGGUCUCUAUGGCCGCACA | 133 | UGUGCGGCCAUAGAGACCCAGAU | 246 |
| 17Q | CUGGGUCUCUAUGGCCGCACA | 133 | UGUGCGGCCAUAGAGACCCAGGC | 247 |
| 18R | CUGGGUCUCUAUGGCCGCACA | 133 | UGUGCGGCCAUAGAGACCCAGAA | 248 |
| 19S | CUAGGUCUCUAUGGCCGCACA | 135 | UGUGCGGCCAUAGAGACCCAGGC | 249 |
| 20T | CUGGGUCUCUAUGGCCGCACA | 133 | CGCGCGGCCACAGAGACCCAGCG | 250 |
| 21U | CAAGGUCUCUAUGGCCGCACA | 136 | CGCGCGGCCACAGAGACCCCGGC | 251 |
| 22V | CUGGGUCUCUAUGGCCGCACA | 133 | CGCGCGGCCACAGAGACCCAGCA | 252 |
| 23W | CUGGGUCUCUAUGGCCGCACA | 133 | CGCGCGGCCACAGAGACCCAGAC | 253 |
| 24X | CUGGGUCUCUAUGGCCGCACA | 133 | CGCGCGGCCACAGAGACCCAGCC | 254 |
| 25Y | CCCGGUCUCUAUGGCCGCACA | 137 | CGCGCGGCCACAGAGACCGGGGC | 255 |
| 26Z | CUGGGUCUCUAUGGCCGCACA | 133 | CGCGCGGCCACAGAGACCCAGCA | 256 |
| 27AA | CAUGGUCUCUAUGGCCGCACA | 138 | CGCGCGGCCACAGAGACCACGGC | 257 |
| 28BB | CAGGGUCUCUAUGGCCGCACA | 139 | CGCGCGGCCACAGAGACCCCGGC | 258 |
| 29CC | CGAGGUCUCUAUGGCCGCACA | 140 | CGCGCGGCCACAGAGACCCCGGC | 259 |
| 30DD | CGGGGUCUCUAUGGCCGCACA | 141 | CGCGCGGCCACAGAGACCCCGGC | 260 |
| 31EE | CGAGGUCUCUAUGGCCGCACA | 142 | CGCGCGGCCACAGAGACCCCGGC | 261 |
| 32FF | CCGGGAUGCAGCCCAGGAACU | 143 | AGCCCCCGGGCCGCACCCCGGCC | 262 |
| 33GG | CGGGAUGCAGCCCAGGAACUU | 144 | AAGCCCCCGGGCCGCACCCCGGC | 263 |
| 34HH | AGCCCAGGAACUUCGGGCAAA | 145 | CCCGCCCGAAGCCCCCGGGCCGC | 264 |
| 35II | GCCCAGGAACUUCGGGCAAGA | 146 | UCUUGCCCGAAGUUCCUGGGCUG | 265 |
| 36JJ | CAGGAACUUCGGGCAAGCCUA | 147 | UAGGCUUGCCCGAAGUUCCUGGG | 266 |
| 37KK | AGGAACUUCGGGCAAGCCUGA | 148 | UCAGGCUUGCCCGAAGUUCCUGG | 267 |
| 38LL | AACUUCGGGCAAGCCUGUUGA | 149 | UCAACAGGCUUGCCCGAAGUUCC | 268 |
| 39MM | ACUUCGGGCAAGCCUGUUGGA | 150 | UCCAACAGGCUUGCCCGAAGUUC | 269 |
| 40NN | UUCGGGCAAGCCUGUUGGAGA | 151 | UCUCCAACAGGCUUGCCCGAAGU | 270 |
| 41OO | GGCAAGCCUGUUGGAGACUCA | 152 | UGAGUCUCCAACAGGCUUGCCCG | 271 |
| 42PP | CAAGCCUGUUGGAGACUCAGA | 153 | UCUGAGUCUCCAACAGGCUUGCC | 272 |
| 43QQ | AAGCCUGUUGGAGACUCAGAU | 154 | AUCUGAGUCUCCAACAGGCUUGC | 273 |
| 44RR | CCUGUUGGAGACUCAGAUGGA | 155 | UCCAUCUGAGUCUCCAACAGGCU | 274 |
| 45SS | CUGUUGGAGACUCAGAUGGAA | 156 | UUCCAUCUGAGUCUCCAACAGGC | 275 |
| 46TT | GGAGACUCAGAUGGAGGAGGA | 157 | UCCUCCUCCAUCUGAGUCUCCAA | 276 |
| 47UU | GAGACUCAGAUGGAGGAGGAU | 158 | AUCCUCCUCCAUCUGAGUCUCCA | 277 |
| 48VV | AGACUCAGAUGGAGGAGGAUA | 159 | UAUCCUCCUCCAUCUGAGUCUCC | 278 |
| 49WW | GACUCAGAUGGAGGAGGAUAU | 160 | AUAUCCUCCUCCAUCUGAGUCUC | 279 |
| 50XX | ACUCAGAUGGAGGAGGAUAUU | 161 | AAUAUCCUCCUCCAUCUGAGUCU | 280 |
| 51YY | ACUCAGAUGGAGGAGGAUAUA | 162 | UAUAUCCUCCUCCAUCUGAGUCU | 281 |
| 52ZZ | UCAGAUGGAGGAGGAUAUUCU | 163 | AGAAUAUCCUCCUCCAUCUGAGU | 282 |
| 54A3 | GCAGAUGGAGGAGGAUAUUCA | 164 | UGAAUAUCCUCCUCCAUCUGCGU | 283 |
| 56B3 | UCAGAUGGAGGAGGAUAUUCA | 165 | UGAAUAUCCUCCUCCAUCUGAGU | 284 |
| 57C3 | UCAGAUGGAGGAGGAUAUUCC | 166 | GGAAUAUCCUCCUCCAUCUGAGU | 285 |
| 62D3 | CAGAUGGAGGAGGAUAUUCUG | 167 | CAGAAUAUCCUCCUCCAUCUGAG | 286 |
| 63E3 | CAGAUGGAGGAGGAUAUUCUA | 168 | UAGAAUAUCCUCCUCCAUCUGAG | 287 |
| 64F3 | AUGGAGGAGGAUAUUCUGCAG | 169 | CUGCAGAAUAUCCUCCUCCAUCU | 288 |
| 65G3 | AUGGAGGAGGAUAUUCUGCAA | 170 | UUGCAGAAUAUCCUCCUCCAUCU | 289 |
| 69H3 | AUGGAGGAGGCUAUUCUGCAG | 171 | CUGCAGAAUAUCCUCCUCCAUCU | 288 |
| 74I3 | AUGGAGGAGGUUAUUCUGCAG | 172 | CUGCAGAAUAUCCUCCUCCAUCU | 288 |
| 80J3 | AUGGAGGAGGAUAUUCUGCAG | 169 | CUGCAGAAUAUCCUCCUCCAUCUTTT | 290 |
| 81K3 | AUGGAGGAGGGUAUUCUGCAG | 173 | CUGCAGAAUAUCCUCCUCCAUCU | 288 |
| 82L3 | AUGGAGGAGGAUAUUCUGCAG | 169 | CUGCAGAAUAUCCUCCUCCAUCUTT | 291 |
| 86M3 | AGAUGGAGGAGGAUAUUCUGCAG | 174 | CUGCAGAAUAUCCUCCUCCAUCU | 288 |
| 89N3 | CAGAUGGAGGAGGAUAUUCUGCAG | 175 | CUGCAGAAUAUCCUCCUCCAUCU | 288 |
| 90O3 | CCAGAUGGAGGAGGAUAUUCUGCAG | 176 | CUGCAGAAUAUCCUCCUCCAUCU | 288 |
| 91P3 | GGAGGAGGAUAUUCUGCAGCU | 177 | AGCUGCAGAAUAUCCUCCUCCAU | 292 |
| 92Q3 | GCCCAGGCACAGAAGGUGCUA | 178 | UAGCACCUUCUGUGCCUGGGCCA | 293 |
| 93R3 | AGCGGCUAGAAGUCCAGCUGA | 179 | UCAGCUGGACUUCUAGCCGCUGC | 294 |
| 94S3 | GCGGCUAGAAGUCCAGCUGAA | 180 | UUCAGCUGGACUUCUAGCCGCUG | 295 |
| 95T3 | CGGCUAGAAGUCCAGCUGAGA | 181 | UCUCAGCUGGACUUCUAGCCGCU | 296 |
| 96U3 | CCGAGAAUUUGAGGUCUUAAA | 182 | UUUAAGACCUCAAAUUCUCGGUA | 297 |
| 98V3 | CGAGAAUUUGAGGUCUUAAAG | 183 | CUUUAAGACCUCAAAUUCUCGGU | 298 |
| 99W3 | GGAGAAUUUGAGGUCUUAAAA | 184 | UUUUAAGACCUCAAAUUCUCCGU | 299 |
| 100X3 | CGAGAAUUUGAGGUCUUAAAA | 185 | UUUUAAGACCUCAAAUUCUCGGU | 300 |
| 109Y3 | AGAAUUUGAGGUCUUAAAGGA | 186 | UCCUUUAAGACCUCAAAUUCUCG | 301 |
| 110Z3 | GAAUUUGAGGUCUUAAAGGCA | 187 | UGCCUUUAAGACCUCAAAUUCUC | 302 |
| 111a4 | AAUUUGAGGUCUUAAAGGCUA | 188 | UAGCCUUUAAGACCUCAAAUUCU | 303 |
| 112b4 | AUUUGAGGUCUUAAAGGCUCA | 189 | UGAGCCUUUAAGACCUCAAAUUC | 304 |
| 113c4 | AUCUGAGGUCUUAAAGGCUCA | 190 | UGAGCCUUUAAGACCUCAGAUUC | 305 |
| 114d4 | ACCUGAGGUCUUAAAGGCUCA | 191 | UGAGCCUUUAAGACCUCAGGUUC | 306 |
| 115e4 | UUUGAGGUCUUAAAGGCUCAA | 192 | UUGAGCCUUUAAGACCUCAAAUU | 307 |
| 116f4 | GAGGUCUUAAAGGCUCACGCU | 193 | AGCGUGAGCCUUUAAGACCUCAA | 308 |
| 117g4 | GAGGUCUUAAAGGCUCACGCA | 194 | UGCGUGAGCCUUUAAGACCUCAA | 309 |
| 118h4 | GGUCUUAAAGGCUCACGCUGA | 195 | UCAGCGUGAGCCUUUAAGACCUC | 310 |
| 124i4 | UGUCUUAAAGGCUCACGCUGC | 196 | GCAGCGUGAGCCUUUAAGACAUC | 311 |
| 126j4 | GUCUUAAAGGCUCACGCUGAA | 197 | UUCAGCGUGAGCCUUUAAGACCU | 312 |
| 127k4 | UCUUAAAGGCUCACGCUGACA | 198 | UGUCAGCGUGAGCCUUUAAGACC | 313 |
| 128l4 | CCGAAAGGCUCACGCUGACAA | 199 | UUGUCAGCGUGAGCCUUUCGGAC | 314 |
| 129m4 | CUUAAAGGCUCACGCUGACAA | 200 | UUGUCAGCGUGAGCCUUUAAGAA | 315 |
| 130n4 | CACAAAGGCUCACGCUGACAA | 201 | UUGUCAGCGUGAGCCUUUGUGAC | 316 |
| 131o4 | CUUAAAGGCUCACGCUGACAA | 200 | UUGUCAGCGUGAGCCUUUAAGGU | 317 |
| 132p4 | CCUAAAGGCUCACGCUGACAA | 202 | UUGUCAGCGUGAGCCUUUAGGAC | 318 |
| 133q4 | CUUAAAGGCUCACGCUGACAA | 200 | UUGUCAGCGUGAGCCUUUAAGCA | 319 |
| 134r4 | CUUAAAGGCUCACGCUGACAA | 200 | UUGUCAGCGUGAGCCUUUAAGAG | 320 |
| 135s4 | CUGAAAGGCUCACGCUGACAA | 203 | UUGUCAGCGUGAGCCUUUCAGAC | 321 |
| 136t4 | CUUAAAGGCUCACGCUGACAA | 200 | UUGUCAGCGUGAGCCUUUAAGGG | 322 |
| 137u4 | CUCAAAGGCUCACGCUGACAA | 204 | UUGUCAGCGUGAGCCUUUGAGAC | 323 |
| 138v4 | CCAAAAGGCUCACGCUGACAA | 205 | UUGUCAGCGUGAGCCUUUUGGAC | 324 |
| 139w4 | CUUAAAGGCUCACGCUGACAA | 200 | UUGUCAGCGUGAGCCUUUAAGUA | 325 |
| 140x4 | CUUAAAGGCUCACGCUGACAA | 200 | UUGUCAGCGUGAGCCUUUAAGGG | 326 |
| 141y4 | CUUAAAGGCUCACGCUGACAA | 200 | UUGUCAGCGUGAGCCUUUAAGGU | 327 |
| 142z4 | CUUAAAGGCUCACGCUGACAA | 200 | UUGUCAGCGUGAGCCUUUAAGGA | 328 |
| 143A5 | CUUAAAGGCUCACGCUGACAA | 200 | UUGUCAGCGUGAGCCUUUAAGUG | 329 |
| 144B5 | CAAAAAGGCUCACGCUGACAA | 206 | UUGUCAGCGUGAGCCUUUUUGAC | 330 |
| 145C5 | CGCAAAGGCUCACGCUGACAA | 207 | UUGUCAGCGUGAGCCUUUGCGAC | 331 |
| 146D5 | CGAAAAGGCUCACGCUGACAA | 208 | UUGUCAGCGUGAGCCUUUUGGAC | 332 |
| 147E5 | CUUAAAGGCUCACGCUGACAA | 200 | UUGUCAGCGUGAGCCUUUAAGGG | 333 |
| 148F5 | CAGAAAGGCUCACGCUGACAA | 209 | UUGUCAGCGUGAGCCUUUGUGAC | 334 |
| 149G5 | CCCAAAGGCUCACGCUGACAA | 210 | UUGUCAGCGUGAGCCUUUGGGAC | 335 |
| 150H5 | CUUAAAGGCUCACGCUGACAA | 200 | UUGUCAGCGUGAGCCUUUAAGAG | 336 |
| 151I5 | CUUAAAGGCUCACGCUGACAA | 200 | UUGUCAGCGUGAGCCUUUAAGCC | 337 |
| 152J5 | CUUAAAGGCUCACGCUGACAA | 200 | UUGUCAGCGUGAGCCUUUAAGAU | 338 |
| 153K5 | CGUAAAGGCUCACGCUGACAA | 211 | UUGUCAGCGUGAGCCUUUACGAC | 339 |
| 154L5 | CUUAAAGGCUCACGCUGACAA | 200 | UUGUCAGCGUGAGCCUUUAAGUC | 340 |
| 155M5 | CUUAAAGGCUCACGCUGACAA | 200 | UUGUCAGCGUGAGCCUUUAAGUU | 341 |
| 156N5 | CGGAAAGGCUCACGCUGACAA | 212 | UUGUCAGCGUGAGCCUUUCCGAC | 342 |
| 157O5 | UUAAAGGCUCACGCUGACAAA | 213 | UUUGUCAGCGUGAGCCUUUAAGA | 343 |
| 158P5 | AGGCUCACGCUGACAAGCAGA | 214 | UCUGCUUGUCAGCGUGAGCCUUU | 344 |
| 159Q5 | UGCAGCGGCAGAGGCGGGAGA | 215 | UCUCCCGCCUCUGCCGCUGCACG | 345 |
| 160R5 | GCGGCAGAGGCGGGAGAUGGU | 216 | ACCAUCUCCCGCCUCUGCCGCUG | 346 |
| 161S5 | GAGGCGGGAGAUGGUGGCACA | 217 | UGUGCCACCAUCUCCCGCCUCUG | 347 |
| 162T5 | GCGGGAGAUGGUGGCACAGCA | 218 | UGCUGUGCCACCAUCUCCCGCCU | 348 |
| 163U5 | GUGGCACAGCAGCAUCGGCUA | 219 | UAGCCGAUGCUGCUGUGCCACCA | 349 |
| 164V5 | CAGCAGCAUCGGCUGCGACAA | 220 | UUGUCGCAGCCGAUGCUGCUGUG | 350 |
| 165W5 | AGCAGCAUCGGCUGCGACAGA | 221 | UCUGUCGCAGCCGAUGCUGCUGU | 351 |
| 166X5 | GCAGCAUCGGCUGCGACAGAA | 222 | UUCUGUCGCAGCCGAUGCUGCUG | 352 |
| 167Y5 | CAGCAUCGGCUGCGACAGAUA | 223 | UAUCUGUCGCAGCCGAUGCUGCU | 353 |
| 168Z5 | AGCAUCGGCUGCGACAGAUCA | 224 | UGAUCUGUCGCAGCCGAUGCUGC | 354 |
| 169a6 | GCAUCGGCUGCGACAGAUCCA | 225 | UGGAUCUGUCGCAGCCGAUGCUG | 355 |
| 170b6 | CAUCGGCUGCGACAGAUCCAA | 226 | UUGGAUCUGUCGCAGCCGAUGCU | 356 |
| 171c6 | UCGGCUGCGACAGAUCCAGGA | 227 | UCCUGGAUCUGUCGCAGCCGAUG | 357 |
| 172d6 | GGCUGCGACAGAUCCAGGAGA | 228 | UCUCCUGGAUCUGUCGCAGCCGA | 358 |
| 173e6 | GCUGCGACAGAUCCAGGAGAA | 229 | UUCUCCUGGAUCUGUCGCAGCCG | 359 |
| 174e7 | CUGCGACAGAUCCAGGAGAGA | 230 | UCUCUCCUGGAUCUGUCGCAGCC | 360 |

What is claimed is:

1. An RNA interference (RNAi) agent comprising a delivery moiety of Formula I: 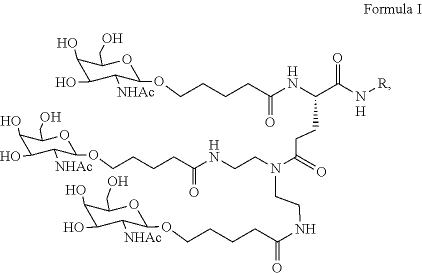
wherein R comprises a sense strand and an antisense strand,
wherein the antisense strand comprises at least 15 contiguous nucleotides of a sequence selected from the group consisting of SEQ ID NOs: 405-525, and wherein the sense strand and the antisense strand form a region of complementarity of at least 15 nucleotides, and wherein the sense strand and antisense strand are each independently 18 to 23 nucleotides in length, and optionally wherein the sense strand and antisense strand each independently comprise one or more modified nucleotides, and optionally wherein the sense strand and the antisense strand each independently comprise one or more modified internucleotide linkages, and wherein R is optionally conjugated to Formula I via a linker.

2. The RNAi agent of claim 1, wherein the antisense strand comprises at least 18 contiguous nucleotides of a sequence selected from the group consisting of SEQ ID NOs: 405-525.

3. The RNAi agent of claim 2, wherein the antisense strand comprises at least 18 contiguous nucleotides of a sequence selected from the group consisting of SEQ ID NOs: 405, 408, 412, 413, 414, 415, 418, 420, 425, 426, 428, 429, 432, 433, 434, 435, 436, 437, 438, 439, 440, 441, 442, 443, 444, 445, 446, 448, 449, 451, 452, 454, 457, 458, 459, 463, 464, 465, 466, 467, 468, 469, 471, 472, 473, 474, 475, 476, 479, 490, 491, 492, 493, 495, 499, 500, 501,502, 503, 504, 505, 506, 507, 508, and 509.

4. The RNAi agent of claim 3, wherein the antisense strand is 23 nucleotides in length.

5. The RNAi agent of claim 4, wherein the sense strand is 21 nucleotides in length.

6. The RNAi agent of claim 5, wherein the antisense strand is selected from the group consisting of SEQ ID NOs: 231-361, or a sequence having at least 90% sequence identity thereto.

7. The RNAi agent of claim 6, wherein the sense strand is selected from the group consisting of SEQ ID NOs: 124-230, or a sequence having at least 90% sequence identity thereto.

8. The RNAi agent of claim 7, wherein, in the region of complementarity comprises 0, 1, 2, or 3 mismatches between the sense strand and the antisense strand.

9. The RNAi agent of claim 8, wherein the sense strand and the antisense strand each independently comprise one or more modified nucleotides and the one or more modified nucleotides are independently 2′ fluoro modified nucleotides or 2′-O-methyl modified nucleotides.

10. The RNAi agent of claim 9, wherein each nucleotide of the sense strand and each nucleotide of the antisense strand is a modified nucleotide.

11. The RNAi agent of claim 10, wherein the sense strand and antisense strand each independently comprise one or more modified internucleotide linkages, and wherein each modified internucleotide linkage is a phosphorothioate linkage.

12. The RNAi agent of claim 11, wherein the sense strand and antisense strand each independently comprise four phosphorothioate linkages.

13. The RNAi agent of claim 12, wherein the 5′ nucleotide of the antisense strand comprises a phosphate group or a phosphate analog.

14. The RNAi agent of claim 13, wherein the antisense strand comprises a sequence selected from the group consisting of SEQ ID NOs: 367-372 and 389-404, or a sequence having at least 90% sequence identity thereto.

15. The RNAi agent of claim 14, wherein the sense strand comprises a sequence selected from the group consisting of SEQ ID NOs: 361-366 and 373-388, or a sequence having at least 90% sequence identity thereto.

16. The RNAi agent of claim 15, wherein the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto;
b. a sense strand having the sequence set forth in SEQ ID NO:362, or a sequence having at least 90% sequence identity thereto, and the antisense strand having the sequence set forth in SEQ ID NO:368, or a sequence having at least 90% sequence identity thereto;
c. a sense strand having the sequence set forth in SEQ ID NO:363, or a sequence having at least 90% sequence identity thereto, and the antisense strand having the sequence set forth in SEQ ID NO:369, or a sequence having at least 90% sequence identity thereto;
d. a sense strand having the sequence set forth in SEQ ID NO:364, or a sequence having at least 90% sequence identity thereto, and the antisense strand having the sequence set forth in SEQ ID NO:370, or a sequence having at least 90% sequence identity thereto;
e. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto;
f. a sense strand having the sequence set forth in SEQ ID NO:366, or a sequence having at least 90% sequence identity thereto, and the antisense strand having the sequence set forth in SEQ ID NO:372, or a sequence having at least 90% sequence identity thereto;
g. a sense strand having the sequence set forth in SEQ ID NO:373, or a sequence having at least 90% sequence identity thereto, and the antisense strand having the sequence set forth in SEQ ID NO:389, or a sequence having at least 90% sequence identity thereto;
h. a sense strand having the sequence set forth in SEQ ID NO:374, or a sequence having at least 90% sequence identity thereto, and an antisense strand having the sequence set forth in SEQ ID NO: 390 or a sequence having at least 90% sequence identity thereto;
i. a sense strand having the sequence set forth in SEQ ID NO:375, or a sequence having at least 90% sequence identity thereto, and an antisense strand having the sequence set forth in SEQ ID NO:391, or a sequence having at least 90% sequence identity thereto;
j. a sense strand having the sequence set forth in SEQ ID NO:376, or a sequence having at least 90% sequence identity thereto, and an antisense strand having the sequence set forth in SEQ ID NO:392, or a sequence having at least 90% sequence identity thereto;
k. a sense strand having the sequence set forth in SEQ ID NO:377, or a sequence having at least 90% sequence identity thereto, and an antisense strand having the sequence set forth in SEQ ID NO:393, or a sequence having at least 90% sequence identity thereto;
l. a sense strand having the sequence set forth in SEQ ID NO:378, or a sequence having at least 90% sequence identity thereto, and an antisense strand having the sequence set forth in SEQ ID NO:394, or a sequence having at least 90% sequence identity thereto;
m. a sense strand having the sequence set forth in SEQ ID NO:379, or a sequence having at least 90% sequence identity thereto, and an antisense strand having the sequence set forth in SEQ ID NO:395, or a sequence having at least 90% sequence identity thereto;
n. a sense strand having the sequence set forth in SEQ ID NO:380 or a sequence having at least 90% sequence identity thereto, and an antisense strand having the sequence set forth in SEQ ID NO:396, or a sequence having at least 90% sequence identity thereto;
o. a sense strand having the sequence set forth in SEQ ID NO: 381, or a sequence having at least 90% sequence identity thereto, and an antisense strand having the sequence set forth in SEQ ID NO:396, or a sequence having at least 90% sequence identity thereto;
p. a sense strand having the sequence set forth in SEQ ID NO:382, or a sequence having at least 90% sequence identity thereto, and an antisense strand having the sequence set forth in SEQ ID NO:397, or a sequence having at least 90% sequence identity thereto;
q. a sense strand having the sequence set forth in SEQ ID NO:383, or a sequence having at least 90% sequence identity thereto, and an antisense strand having the sequence set forth in SEQ ID NO:398, or a sequence having at least 90% sequence identity thereto;
r. a sense strand having the sequence set forth in SEQ ID NO:384, or a sequence having at least 90% sequence identity thereto, and an antisense strand having the sequence set forth in SEQ ID NO:399, or a sequence having at least 90% sequence identity thereto;
s. a sense strand having the sequence set forth in SEQ ID NO:385, or a sequence having at least 90% sequence identity thereto, and an antisense strand having the sequence set forth in SEQ ID NO:400, or a sequence having at least 90% sequence identity thereto;
t. a sense strand having the sequence set forth in SEQ ID NO:386, or a sequence having at least 90% sequence identity thereto, and an antisense strand having the sequence set forth in SEQ ID NO:401, or a sequence having at least 90% sequence identity thereto;
u. a sense strand having the sequence set forth in SEQ ID NO:387, or a sequence having at least 90% sequence identity thereto, and an antisense strand having the sequence set forth in SEQ ID NO:402, or a sequence having at least 90% sequence identity thereto;
v. a sense strand having the sequence set forth in SEQ ID NO:388, or a sequence having at least 90% sequence identity thereto, and an antisense strand having the sequence set forth in SEQ ID NO:403, or a sequence having at least 90% sequence identity thereto; and
w. a sense strand having the sequence set forth in SEQ ID NO:389, or a sequence having at least 90% sequence identity thereto, and an antisense strand having the sequence set forth in SEQ ID NO:404, or a sequence having at least 90% sequence identity thereto.

17. The RNAi agent of claim 15, wherein the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto;
b. a sense strand having the sequence set forth in SEQ ID NO:362, or a sequence having at least 95% sequence identity thereto, and the antisense strand having the sequence set forth in SEQ ID NO:368, or a sequence having at least 95% sequence identity thereto;
c. a sense strand having the sequence set forth in SEQ ID NO:363, or a sequence having at least 95% sequence identity thereto, and the antisense strand having the sequence set forth in SEQ ID NO:369, or a sequence having at least 95% sequence identity thereto;
d. a sense strand having the sequence set forth in SEQ ID NO:364, or a sequence having at least 95% sequence identity thereto, and the antisense strand having the sequence set forth in SEQ ID NO:370, or a sequence having at least 95% sequence identity thereto;
e. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto;
f. a sense strand having the sequence set forth in SEQ ID NO:366, or a sequence having at least 95% sequence identity thereto, and the antisense strand having the sequence set forth in SEQ ID NO:372, or a sequence having at least 95% sequence identity thereto;
g. a sense strand having the sequence set forth in SEQ ID NO:373, or a sequence having at least 95% sequence identity thereto, and the antisense strand having the sequence set forth in SEQ ID NO:389, or a sequence having at least 95% sequence identity thereto;
h. a sense strand having the sequence set forth in SEQ ID NO:374, or a sequence having at least 95% sequence identity thereto, and an antisense strand having the sequence set forth in SEQ ID NO: 390 or a sequence having at least 95% sequence identity thereto;
i. a sense strand having the sequence set forth in SEQ ID NO:375, or a sequence having at least 95% sequence identity thereto, and an antisense strand having the sequence set forth in SEQ ID NO:391, or a sequence having at least 95% sequence identity thereto;
j. a sense strand having the sequence set forth in SEQ ID NO:376, or a sequence having at least 95% sequence identity thereto, and an antisense strand having the sequence set forth in SEQ ID NO:392, or a sequence having at least 95% sequence identity thereto;
k. a sense strand having the sequence set forth in SEQ ID NO:377, or a sequence having at least 95% sequence identity thereto, and an antisense strand having the sequence set forth in SEQ ID NO:393, or a sequence having at least 95% sequence identity thereto;
l. a sense strand having the sequence set forth in SEQ ID NO:378, or a sequence having at least 95% sequence identity thereto, and an antisense strand having the sequence set forth in SEQ ID NO:394, or a sequence having at least 95% sequence identity thereto;
m. a sense strand having the sequence set forth in SEQ ID NO:379, or a sequence having at least 95% sequence identity thereto, and an antisense strand having the sequence set forth in SEQ ID NO:395, or a sequence having at least 95% sequence identity thereto;
n. a sense strand having the sequence set forth in SEQ ID NO:380 or a sequence having at least 95% sequence identity thereto, and an antisense strand having the sequence set forth in SEQ ID NO:396, or a sequence having at least 95% sequence identity thereto;
o. a sense strand having the sequence set forth in SEQ ID NO: 381, or a sequence having at least 95% sequence identity thereto, and an antisense strand having the sequence set forth in SEQ ID NO:396, or a sequence having at least 95% sequence identity thereto;
p. a sense strand having the sequence set forth in SEQ ID NO:382, or a sequence having at least 95% sequence identity thereto, and an antisense strand having the sequence set forth in SEQ ID NO:397, or a sequence having at least 95% sequence identity thereto;
q. a sense strand having the sequence set forth in SEQ ID NO:383, or a sequence having at least 95% sequence identity thereto, and an antisense strand having the sequence set forth in SEQ ID NO:398, or a sequence having at least 95% sequence identity thereto;
r. a sense strand having the sequence set forth in SEQ ID NO:384, or a sequence having at least 95% sequence identity thereto, and an antisense strand having the sequence set forth in SEQ ID NO:399, or a sequence having at least 95% sequence identity thereto;
s. a sense strand having the sequence set forth in SEQ ID NO:385, or a sequence having at least 95% sequence identity thereto, and an antisense strand having the sequence set forth in SEQ ID NO:400, or a sequence having at least 95% sequence identity thereto;
t. a sense strand having the sequence set forth in SEQ ID NO:386, or a sequence having at least 95% sequence identity thereto, and an antisense strand having the sequence set forth in SEQ ID NO:401, or a sequence having at least 95% sequence identity thereto;
u. a sense strand having the sequence set forth in SEQ ID NO:387, or a sequence having at least 95% sequence identity thereto, and an antisense strand having the sequence set forth in SEQ ID NO:402, or a sequence having at least 95% sequence identity thereto;
v. a sense strand having the sequence set forth in SEQ ID NO:388, or a sequence having at least 95% sequence identity thereto, and an antisense strand having the sequence set forth in SEQ ID NO:403, or a sequence having at least 95% sequence identity thereto; and
w. a sense strand having the sequence set forth in SEQ ID NO:389, or a sequence having at least 95% sequence identity thereto, and an antisense strand having the sequence set forth in SEQ ID NO:404, or a sequence having at least 95% sequence identity thereto.

18. The RNAi agent of claim 16, wherein the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto;
b. a sense strand having the sequence set forth in SEQ ID NO:362, or a sequence having at least 90% sequence identity thereto, and the antisense strand having the sequence set forth in SEQ ID NO:368, or a sequence having at least 90% sequence identity thereto;
c. a sense strand having the sequence set forth in SEQ ID NO:363, or a sequence having at least 90% sequence identity thereto, and the antisense strand having the sequence set forth in SEQ ID NO:369, or a sequence having at least 90% sequence identity thereto;
d. a sense strand having the sequence set forth in SEQ ID NO:364, or a sequence having at least 90% sequence identity thereto, and the antisense strand having the sequence set forth in SEQ ID NO:370, or a sequence having at least 90% sequence identity thereto; and
e. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto.

19. The RNAi agent of claim 17, wherein the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto;
b. a sense strand having the sequence set forth in SEQ ID NO:362, or a sequence having at least 95% sequence identity thereto, and the antisense strand having the sequence set forth in SEQ ID NO:368, or a sequence having at least 95% sequence identity thereto;
c. a sense strand having the sequence set forth in SEQ ID NO:363, or a sequence having at least 95% sequence identity thereto, and the antisense strand having the sequence set forth in SEQ ID NO:369, or a sequence having at least 95% sequence identity thereto;
d. a sense strand having the sequence set forth in SEQ ID NO:364, or a sequence having at least 95% sequence identity thereto, and the antisense strand having the sequence set forth in SEQ ID NO:370, or a sequence having at least 95% sequence identity thereto; and
e. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto.

20. The RNAi agent of claim 18, wherein the sense strand and antisense strand are a pair of oligonucleotide sequences selected from the group consisting of:
a. a sense strand having the sequence set forth in SEQ ID NO:361, or a sequence having at least 90% sequence identity thereto, and the antisense strand having the sequence set forth in SEQ ID NO:367, or a sequence having at least 90% sequence identity thereto; and
b. a sense strand having the sequence set forth in SEQ ID NO:365, or a sequence having at least 90% sequence identity thereto, and the antisense strand having the sequence set forth in SEQ ID NO:371, or a sequence having at least 90% sequence identity thereto.

21. The RNAi agent of claim 19, wherein the sense strand and antisense strand are a pair of oligonucleotide sequences selected from the group consisting of:
a. a sense strand having the sequence set forth in SEQ ID NO:361, or a sequence having at least 95% sequence identity thereto, and the antisense strand having the sequence set forth in SEQ ID NO:367, or a sequence having at least 95% sequence identity thereto; and
b. a sense strand having the sequence set forth in SEQ ID NO:365, or a sequence having at least 95% sequence identity thereto, and the antisense strand having the sequence set forth in SEQ ID NO:371, or a sequence having at least 95% sequence identity thereto.

22. The RNAi agent of claim 21, wherein R is conjugated to Formula I via a linker.

23. The RNAi agent of claim 22, wherein R is conjugated to Formula I via a linker, and wherein linker comprises a linker of Formula II having connection points A and B or the linker comprises Formula III having connection points C and D, and wherein connection point A or connection point C is conjugated to Formula I and connection point B or connection point D is conjugated to a phosphate group which is conjugated to R;

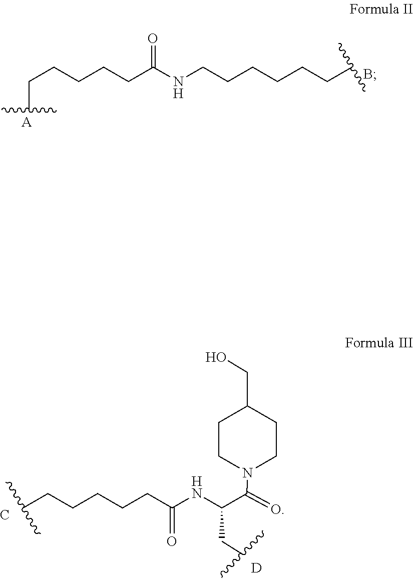

24. The RNAi agent of claim 23, wherein R is conjugated to Formula I via a linker, and wherein the linker is a linker comprising Formula III having connection points C and D, and wherein connection point C is conjugated to Formula I and connection point D is conjugated to a phosphate group which is conjugated to R;

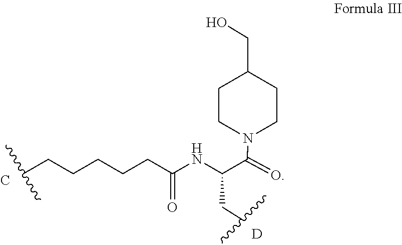

25. The RNAi agent of claim 24, wherein the sense strand and the antisense strand are a pair of oligonucleotide sequences selected from the group consisting of:
a. a sense strand consisting of the sequence set forth in SEQ ID NO:361 and an antisense strand consisting of the sequence set forth in SEQ ID NO:367; and
b. a sense strand consisting of the sequence set forth in SEQ ID NO:365 and an antisense strand consisting of the sequence set forth in SEQ ID NO:371.

26. The RNAi agent of claim 25, wherein the RNAi agent is capable of decreasing expression of the ANGPTL8 gene in a liver cell.

27. A method of treating dyslipidemia in a patient in need thereof, comprising administering the RNAi agent of claim 1.


SEQUENCE LISTING (525 sequences)

SEQ ID NO: 1 - DNA, 872 bp, HOMO SAPIENS
ataccttagaccctcagtcatgccagtgcctgctctgtgcctgctctgggccctggcaatggtgacccggcctgcctcagcggcccccatgggcggcccagaactggcacagcatgaggagctgaccctgctcttccatgggaccctgcagctgggccaggccctcaacggtgtgtacaggaccacggagggacggctgacaaaggccaggaacagcctgggtctctatggccgcacaatagaactcctggggcaggaggtcagccggggccgggatgcagcccaggaacttcgggcaagcctgttggagactcagatggaggaggatattctgcagctgcaggcagaggccacagctgaggtgctgggggaggtggcccaggcacagaaggtgctacgggacagcgtgcagcggctagaagtccagctgaggagcgcctggctgggccctgcctaccgagaatttgaggtcttaaaggctcacgctgacaagcagagccacatcctatgggccctcacaggccacgtgcagcggcagaggcgggagatggtggcacagcagcatcggctgcgacagatccaggagagactccacacagcggcgctcccagcctgaatctgcctggatggaactgaggaccaatcatgctgcaaggaacacttccacgccccgtgaggcccctgtgcagggaggagctgcctgttcactgggatcagccagggcgccgggccccacttctgagcacagagcagagacagacgcaggcggggacaaaggcagaggatgtagccccattggggaggggtggaggaaggacatgtaccctttcatgcctacacacccctcattaaagcagagtcgtggcatctca

SEQ ID NO: 2 - DNA, 1386 bp, HOMO SAPIENS
atgttcaccatcaagctgctgctgttcatcgtgcccctcgtgatcagcagcagaatcgaccaggacaacagcagcttcgacagcctgagccccgagcccaagagcagattcgccatgctggacgacgtgaagatcctggccaacggcctgctgcagctgggccacggcctgaaggatttcgtgcacaagaccaagggccagatcaacgacatcttccagaagctgaacatcttcgaccagagcttctacgacctgagcctgcagaccagcgagatcaaagaggaagagaaagagctgcggaggaccacctacaagctgcaagtgaagaacgaggaagtgaaaaacatgagcctggaactgaacagcaagctggaaagcctgctggaagaaaagattctgctgcagcagaaagtgaagtacctggaagaacagctgaccaacctgatccagaaccagcccgagacacccgagcaccccgaagtgaccagcctgaaaaccttcgtggaaaagcaggacaactccatcaaggacctgctgcagaccgtggaagatcagtacaagcagctgaaccagcagcactcccagatcaaagaaatcgagaaccagctgaggcggaccagcatccaggaacccaccgagatcagcctgtccagcaagcccagagcccccagaacaaccccattcctgcagctgaatgagatccggaacgtgaagcacgacggcatccctgccgagtgcaccaccatctacaacagaggcgagcacaccagcgggatgtacgccatcagacccagcaacagccaggtgttccacgtgtactgcgacgtgatcagcggcagcccctggacactgatccagcacagaatcgatggcagccagaacttcaacgagacatgggagaactataagtacggcttcggcagactggacggcgagttttggctgggcctggaaaagatctacagcatcgtgaagcagagcaactacgtgctgagaatcgagctggaagattggaaggacaacaagcactacatcgagtacagcttctacctgggcaaccacgagacaaactacaccctgcacctggtggccatcaccggcaacgtgccaaacgccatccccgagaacaaggatctggtgttcagcacctgggaccacaaggctaagggccacttcaactgccccgagggctactctggcggctggtggtggcatgatgagtgcggcgagaacaacctgaacggcaagtacaacaagcccagggccaagagcaagcctgagagaagaagaggcctgtcctggaagtcccagaacggcaggctgtactctatcaagagcaccaagatgctgatccaccccaccgacagcgagagcttcgagtgataa

SEQ ID NO: 3 - DNA, 18 bp, HOMO SAPIENS
agctgaccctgctcttcc

SEQ ID NO: 4 - DNA, 18 bp, HOMO SAPIENS
ggaccctgcagctgggcc

SEQ ID NO: 5 - DNA, 18 bp, HOMO SAPIENS
agctgggccaggccctca

SEQ ID NO: 6 - DNA, 18 bp, HOMO SAPIENS
acggagggacggctgaca

SEQ ID NO: 7 - DNA, 18 bp, HOMO SAPIENS
cggagggacggctgacaa

SEQ ID NO: 8 - DNA, 18 bp, HOMO SAPIENS
ggacggctgacaaaggcc

SEQ ID NO: 9 - DNA, 18 bp, HOMO SAPIENS
cggctgacaaaggccagg

SEQ ID NO: 10 - DNA, 18 bp, HOMO SAPIENS
ggctgacaaaggccagga

SEQ ID NO: 11 - DNA, 18 bp, HOMO SAPIENS
ctgacaaaggccaggaac

SEQ ID NO: 12 - DNA, 18 bp, HOMO SAPIENS
ggaacagcctgggtctct

SEQ ID NO: 13 - DNA, 18 bp, HOMO SAPIENS
gaacagcctgggtctcta

SEQ ID NO: 14 - DNA, 18 bp, HOMO SAPIENS
agcctgggtctctatggc

SEQ ID NO: 15 - DNA, 18 bp, HOMO SAPIENS
cctgggtctctatggccg

SEQ ID NO: 16 - DNA, 18 bp, HOMO SAPIENS
ctgggtctctatggccgc

SEQ ID NO: 17 - DNA, 18 bp, HOMO SAPIENS
tgggtctctatggccgca

SEQ ID NO: 18 - DNA, 18 bp, HOMO SAPIENS
gggtctctatggccgcac

SEQ ID NO: 19 - DNA, 18 bp, HOMO SAPIENS
ggtcagccggggccggga

SEQ ID NO: 20 - DNA, 18 bp, HOMO SAPIENS
ggggccgggatgcagccc

SEQ ID NO: 21 - DNA, 18 bp, HOMO SAPIENS
gccgggatgcagcccagg

SEQ ID NO: 22 - DNA, 18 bp, HOMO SAPIENS
ccgggatgcagcccagga

SEQ ID NO: 23 - DNA, 18 bp, HOMO SAPIENS
gggatgcagcccaggaac

SEQ ID NO: 24 - DNA, 18 bp, HOMO SAPIENS
ggatgcagcccaggaact

SEQ ID NO: 25 - DNA, 18 bp, HOMO SAPIENS
gcccaggaacttcgggca

SEQ ID NO: 26 - DNA, 18 bp, HOMO SAPIENS
cccaggaacttcgggcaa

SEQ ID NO: 27 - DNA, 18 bp, HOMO SAPIENS
ccaggaacttcgggcaag

SEQ ID NO: 28 - DNA, 18 bp, HOMO SAPIENS
caggaacttcgggcaagc

SEQ ID NO: 29 - DNA, 18 bp, HOMO SAPIENS
aggaacttcgggcaagcc

SEQ ID NO: 30 - DNA, 18 bp, HOMO SAPIENS
ggaacttcgggcaagcct

SEQ ID NO: 31 - DNA, 18 bp, HOMO SAPIENS
gaacttcgggcaagcctg

SEQ ID NO: 32 - DNA, 18 bp, HOMO SAPIENS
cttcgggcaagcctgttg

SEQ ID NO: 33 - DNA, 18 bp, HOMO SAPIENS
ttcgggcaagcctgttgg

SEQ ID NO: 34 - DNA, 18 bp, HOMO SAPIENS
cgggcaagcctgttggag

SEQ ID NO: 35 - DNA, 18 bp, HOMO SAPIENS
caagcctgttggagactc

SEQ ID NO: 36 - DNA, 18 bp, HOMO SAPIENS
agcctgttggagactcag

SEQ ID NO: 37 - DNA, 18 bp, HOMO SAPIENS
gcctgttggagactcaga

SEQ ID NO: 38 - DNA, 18 bp, HOMO SAPIENS
tgttggagactcagatgg

SEQ ID NO: 39 - DNA, 18 bp, HOMO SAPIENS
gttggagactcagatgga

SEQ ID NO: 40 - DNA, 18 bp, HOMO SAPIENS
agactcagatggaggagg

SEQ ID NO: 41 - DNA, 18 bp, HOMO SAPIENS
gactcagatggaggagga

SEQ ID NO: 42 - DNA, 18 bp, HOMO SAPIENS
actcagatggaggaggat

SEQ ID NO: 43 - DNA, 18 bp, HOMO SAPIENS
ctcagatggaggaggata

SEQ ID NO: 44 - DNA, 18 bp, HOMO SAPIENS
tcagatggaggaggatat

SEQ ID NO: 45 - DNA, 18 bp, HOMO SAPIENS
cagatggaggaggatatt

SEQ ID NO: 46 - DNA, 18 bp, HOMO SAPIENS
agatggaggaggatattc

SEQ ID NO: 47 - DNA, 18 bp, HOMO SAPIENS
gatggaggaggatattct

SEQ ID NO: 48 - DNA, 18 bp, HOMO SAPIENS
atggaggaggatattctg

SEQ ID NO: 49 - DNA, 18 bp, HOMO SAPIENS
ggaggaggatattctgca

SEQ ID NO: 50 - DNA, 18 bp, HOMO SAPIENS
aggaggatattctgcagc

SEQ ID NO: 51 - DNA, 18 bp, HOMO SAPIENS
aggtggcccaggcacaga

SEQ ID NO: 52 - DNA, 18 bp, HOMO SAPIENS
ccaggcacagaaggtgct

SEQ ID NO: 53 - DNA, 18 bp, HOMO SAPIENS
agcggctagaagtccagc

SEQ ID NO: 54 - DNA, 18 bp, HOMO SAPIENS
gcggctagaagtccagct

SEQ ID NO: 55 - DNA, 18 bp, HOMO SAPIENS
cggctagaagtccagctg

SEQ ID NO: 56 - DNA, 18 bp, HOMO SAPIENS
ggctagaagtccagctga

SEQ ID NO: 57 - DNA, 18 bp, HOMO SAPIENS
gctagaagtccagctgag

SEQ ID NO: 58 - DNA, 18 bp, HOMO SAPIENS
aagtccagctgaggagcg

SEQ ID NO: 59 - DNA, 18 bp, HOMO SAPIENS
agtccagctgaggagcgc

SEQ ID NO: 60 - DNA, 18 bp, HOMO SAPIENS
cctggctgggccctgcct

SEQ ID NO: 61 - DNA, 18 bp, HOMO SAPIENS
gagaatttgaggtcttaa

SEQ ID NO: 62 - DNA, 18 bp, HOMO SAPIENS
agaatttgaggtcttaaa

SEQ ID NO: 63 - DNA, 18 bp, HOMO SAPIENS
aatttgaggtcttaaagg

SEQ ID NO: 64 - DNA, 18 bp, HOMO SAPIENS
atttgaggtcttaaaggc

SEQ ID NO: 65 - DNA, 18 bp, HOMO SAPIENS
tttgaggtcttaaaggct

SEQ ID NO: 66 - DNA, 18 bp, HOMO SAPIENS
ttgaggtcttaaaggctc

SEQ ID NO: 67 - DNA, 18 bp, HOMO SAPIENS
tgaggtcttaaaggctca

SEQ ID NO: 68 - DNA, 18 bp, HOMO SAPIENS
gaggtcttaaaggctcac

SEQ ID NO: 69 - DNA, 18 bp, HOMO SAPIENS
ggtcttaaaggctcacgc

SEQ ID NO: 70 - DNA, 18 bp, HOMO SAPIENS
tcttaaaggctcacgctg

SEQ ID NO: 71 - DNA, 18 bp, HOMO SAPIENS
cttaaaggctcacgctga

SEQ ID NO: 72 - DNA, 18 bp, HOMO SAPIENS
ttaaaggctcacgctgac

SEQ ID NO: 73 - DNA, 18 bp, HOMO SAPIENS
taaaggctcacgctgaca

SEQ ID NO: 74 - DNA, 18 bp, HOMO SAPIENS
aaaggctcacgctgacaa

SEQ ID NO: 75 - DNA, 18 bp, HOMO SAPIENS
aaggctcacgctgacaag

SEQ ID NO: 76 - DNA, 18 bp, HOMO SAPIENS
aggctcacgctgacaagc

SEQ ID NO: 77 - DNA, 18 bp, HOMO SAPIENS
gctcacgctgacaagcag

SEQ ID NO: 78 - DNA, 18 bp, HOMO SAPIENS
ctcacgctgacaagcaga

SEQ ID NO: 79 - DNA, 18 bp, HOMO SAPIENS
tcacgctgacaagcagag

SEQ ID NO: 80 - DNA, 18 bp, HOMO SAPIENS
cacgctgacaagcagagc

SEQ ID NO: 81 - DNA, 18 bp, HOMO SAPIENS
acgctgacaagcagagcc

SEQ ID NO: 82 - DNA, 18 bp, HOMO SAPIENS
gctgacaagcagagccac

SEQ ID NO: 83 - DNA, 18 bp, HOMO SAPIENS
ccctcacaggccacgtgc

SEQ ID NO: 84 - DNA, 18 bp, HOMO SAPIENS
cctcacaggccacgtgca

SEQ ID NO: 85 - DNA, 18 bp, HOMO SAPIENS
aggccacgtgcagcggca

SEQ ID NO: 86 - DNA, 18 bp, HOMO SAPIENS
ggccacgtgcagcggcag

SEQ ID NO: 87 - DNA, 18 bp, HOMO SAPIENS
cacgtgcagcggcagagg

SEQ ID NO: 88 - DNA, 18 bp, HOMO SAPIENS
cagcggcagaggcgggag

SEQ ID NO: 89 - DNA, 18 bp, HOMO SAPIENS
ggcagaggcgggagatgg

SEQ ID NO: 90 - DNA, 18 bp, HOMO SAPIENS
ggcgggagatggtggcac

SEQ ID NO: 91 - DNA, 18 bp, HOMO SAPIENS
gggagatggtggcacagc

SEQ ID NO: 92 - DNA, 18 bp, HOMO SAPIENS
agatggtggcacagcagc

SEQ ID NO: 93 - DNA, 18 bp, HOMO SAPIENS
ggcacagcagcatcggct

SEQ ID NO: 94 - DNA, 18 bp, HOMO SAPIENS
cacagcagcatcggctgc

SEQ ID NO: 95 - DNA, 18 bp, HOMO SAPIENS
cagcagcatcggctgcga

SEQ ID NO: 96 - DNA, 18 bp, HOMO SAPIENS
agcagcatcggctgcgac

SEQ ID NO: 97 - DNA, 18 bp, HOMO SAPIENS
gcagcatcggctgcgaca

SEQ ID NO: 98 - DNA, 18 bp, HOMO SAPIENS
cagcatcggctgcgacag

SEQ ID NO: 99 - DNA, 18 bp, HOMO SAPIENS
agcatcggctgcgacaga

SEQ ID NO: 100 - DNA, 18 bp, HOMO SAPIENS
gcatcggctgcgacagat

SEQ ID NO: 101 - DNA, 18 bp, HOMO SAPIENS
catcggctgcgacagatc

SEQ ID NO: 102 - DNA, 18 bp, HOMO SAPIENS
atcggctgcgacagatcc

SEQ ID NO: 103 - DNA, 18 bp, HOMO SAPIENS
tcggctgcgacagatcca

SEQ ID NO: 104 - DNA, 18 bp, HOMO SAPIENS
ggctgcgacagatccagg

SEQ ID NO: 105 - DNA, 18 bp, HOMO SAPIENS
ctgcgacagatccaggag

SEQ ID NO: 106 - DNA, 18 bp, HOMO SAPIENS
tgcgacagatccaggaga

SEQ ID NO: 107 - DNA, 18 bp, HOMO SAPIENS
gcgacagatccaggagag

SEQ ID NO: 108 - DNA, 18 bp, HOMO SAPIENS
cccagcctgaatctgcct

SEQ ID NO: 109 - DNA, 18 bp, HOMO SAPIENS
agcctgaatctgcctgga

SEQ ID NO: 110 - DNA, 18 bp, HOMO SAPIENS
gcctgaatctgcctggat

SEQ ID NO: 111 - DNA, 18 bp, HOMO SAPIENS
ctgcctggatggaactga

SEQ ID NO: 112 - DNA, 18 bp, HOMO SAPIENS
tcatgctgcaaggaacac

SEQ ID NO: 113 - DNA, 18 bp, HOMO SAPIENS
caaggaacacttccacgc

SEQ ID NO: 114 - DNA, 18 bp, HOMO SAPIENS
ggaacacttccacgcccc

SEQ ID NO: 115 - DNA, 18 bp, HOMO SAPIENS
tgcctgttcactgggatc

SEQ ID NO: 116 - DNA, 18 bp, HOMO SAPIENS
gcctgttcactgggatca

SEQ ID NO: 117 - DNA, 18 bp, HOMO SAPIENS
ctgttcactgggatcagc

SEQ ID NO: 118 - DNA, 18 bp, HOMO SAPIENS
tgttcactgggatcagcc

SEQ ID NO: 119 - DNA, 18 bp, HOMO SAPIENS
tgggatcagccagggcgc

SEQ ID NO: 120 - DNA, 18 bp, HOMO SAPIENS
gggatcagccagggcgcc

SEQ ID NO: 121 - DNA, 18 bp, HOMO SAPIENS
cattaaagcagagtcgtg

SEQ ID NO: 122 - DNA, 18 bp, HOMO SAPIENS
attaaagcagagtcgtgg

SEQ ID NO: 123 - DNA, 18 bp, HOMO SAPIENS
taaagcagagtcgtggca

SEQ ID NO: 124 - RNA, 21 nt, ARTIFICIAL SEQUENCE
  Synthetic Oligonucleotide
ggagcugacccugcucuucca

SEQ ID NO: 125 - RNA, 21 nt, ARTIFICIAL SEQUENCE
  Synthetic Oligonucleotide
ccacggagggacggcugacaa

SEQ ID NO: 126 - RNA, 21 nt, ARTIFICIAL SEQUENCE
  Synthetic Oligonucleotide
acggcugacaaaggccaggaa

SEQ ID NO: 127 - RNA, 21 nt, ARTIFICIAL SEQUENCE
  Synthetic Oligonucleotide
ggcugacaaaggccaggaaca

SEQ ID NO: 128 - RNA, 21 nt, ARTIFICIAL SEQUENCE
  Synthetic Oligonucleotide
caggaacagccugggucucua

SEQ ID NO: 129 - RNA, 21 nt, ARTIFICIAL SEQUENCE
  Synthetic Oligonucleotide
caggaacagccugggucucuc

SEQ ID NO: 130 - RNA, 21 nt, ARTIFICIAL SEQUENCE
  Synthetic Oligonucleotide
aggaacagccugggucucuau

SEQ ID NO: 131 - RNA, 21 nt, ARTIFICIAL SEQUENCE
  Synthetic Oligonucleotide
aggaacagccugggucucuaa

SEQ ID NO: 132 - RNA, 21 nt, ARTIFICIAL SEQUENCE
  Synthetic Oligonucleotide
gccugggucucuauggccgca

SEQ ID NO: 133 - RNA, 21 nt, ARTIFICIAL SEQUENCE
  Synthetic Oligonucleotide
cugggucucuauggccgcaca

SEQ ID NO: 134 - RNA, 21 nt, ARTIFICIAL SEQUENCE
  Synthetic Oligonucleotide
cuuggucucuauggccgcaca

SEQ ID NO: 135 - RNA, 21 nt, ARTIFICIAL SEQUENCE
  Synthetic Oligonucleotide
cuaggucucuauggccgcaca

SEQ ID NO: 136 - RNA, 21 nt, ARTIFICIAL SEQUENCE
  Synthetic Oligonucleotide
caaggucucuauggccgcaca

SEQ ID NO: 137 - RNA, 21 nt, ARTIFICIAL SEQUENCE
  Synthetic Oligonucleotide
cccggucucuauggccgcaca

SEQ ID NO: 138 - RNA, 21 nt, ARTIFICIAL SEQUENCE
  Synthetic Oligonucleotide
cauggucucuauggccgcaca

SEQ ID NO: 139 - RNA, 21 nt, ARTIFICIAL SEQUENCE
  Synthetic Oligonucleotide
cagggucucuauggccgcaca

SEQ ID NO: 140 - RNA, 21 nt, ARTIFICIAL SEQUENCE
  Synthetic Oligonucleotide
cgaggucucuauggccgcaca

SEQ ID NO: 141 - RNA, 21 nt, ARTIFICIAL SEQUENCE
  Synthetic Oligonucleotide
cggggucucuauggccgcaca

SEQ ID NO: 142 - RNA, 21 nt, ARTIFICIAL SEQUENCE
  Synthetic Oligonucleotide
ccaggucucuauggccgcaca

SEQ ID NO: 143 - RNA, 21 nt, ARTIFICIAL SEQUENCE
  Synthetic Oligonucleotide
ccgggaugcagcccaggaacu

SEQ ID NO: 144 - RNA, 21 nt, ARTIFICIAL SEQUENCE
  Synthetic Oligonucleotide
cgggaugcagcccaggaacuu

SEQ ID NO: 145 - RNA, 21 nt, ARTIFICIAL SEQUENCE
  Synthetic Oligonucleotide
agcccaggaacuucgggcaaa

SEQ ID NO: 146 - RNA, 21 nt, ARTIFICIAL SEQUENCE
  Synthetic Oligonucleotide
gcccaggaacuucgggcaaga

SEQ ID NO: 147 - RNA, 21 nt, ARTIFICIAL SEQUENCE
  Synthetic Oligonucleotide
caggaacuucgggcaagccua

SEQ ID NO: 148 - RNA, 21 nt, ARTIFICIAL SEQUENCE
  Synthetic Oligonucleotide
aggaacuucgggcaagccuga

SEQ ID NO: 149 - RNA, 21 nt, ARTIFICIAL SEQUENCE
  Synthetic Oligonucleotide
aacuucgggcaagccuguuga

SEQ ID NO: 150 - RNA, 21 nt, ARTIFICIAL SEQUENCE
  Synthetic Oligonucleotide
acuucgggcaagccuguugga

SEQ ID NO: 151 - RNA, 21 nt, ARTIFICIAL SEQUENCE
  Synthetic Oligonucleotide
uucgggcaagccuguuggaga

SEQ ID NO: 152 - RNA, 21 nt, ARTIFICIAL SEQUENCE
  Synthetic Oligonucleotide
ggcaagccuguuggagacuca

SEQ ID NO: 153 - RNA, 21 nt, ARTIFICIAL SEQUENCE
  Synthetic Oligonucleotide
caagccuguuggagacucaga

SEQ ID NO: 154 - RNA, 21 nt, ARTIFICIAL SEQUENCE
  Synthetic Oligonucleotide
aagccuguuggagacucagau

SEQ ID NO: 155 - RNA, 21 nt, ARTIFICIAL SEQUENCE
  Synthetic Oligonucleotide
ccuguuggagacucagaugga

SEQ ID NO: 156 - RNA, 21 nt, ARTIFICIAL SEQUENCE
  Synthetic Oligonucleotide
cuguuggagacucagauggaa

SEQ ID NO: 157 - RNA, 21 nt, ARTIFICIAL SEQUENCE
  Synthetic Oligonucleotide
ggagacucagauggaggagga

SEQ ID NO: 158 - RNA, 21 nt, ARTIFICIAL SEQUENCE
  Synthetic Oligonucleotide
gagacucagauggaggaggau

SEQ ID NO: 159 - RNA, 21 nt, ARTIFICIAL SEQUENCE
  Synthetic Oligonucleotide
agacucagauggaggaggaua

SEQ ID NO: 160 - RNA, 21 nt, ARTIFICIAL SEQUENCE
  Synthetic Oligonucleotide
gacucagauggaggaggauau

SEQ ID NO: 161 - RNA, 21 nt, ARTIFICIAL SEQUENCE
  Synthetic Oligonucleotide
acucagauggaggaggauauu

SEQ ID NO: 162 - RNA, 21 nt, ARTIFICIAL SEQUENCE
  Synthetic Oligonucleotide
acucagauggaggaggauaua

SEQ ID NO: 163 - RNA, 21 nt, ARTIFICIAL SEQUENCE
  Synthetic Oligonucleotide
ucagauggaggaggauauucu

SEQ ID NO: 164 - RNA, 21 nt, ARTIFICIAL SEQUENCE
  Synthetic Oligonucleotide
gcagauggaggaggauauuca

SEQ ID NO: 165 - RNA, 21 nt, ARTIFICIAL SEQUENCE
  Synthetic Oligonucleotide
ucagauggaggaggauauuca

SEQ ID NO: 166 - RNA, 21 nt, ARTIFICIAL SEQUENCE
  Synthetic Oligonucleotide
ucagauggaggaggauauucc

SEQ ID NO: 167 - RNA, 21 nt, ARTIFICIAL SEQUENCE
  Synthetic Oligonucleotide
cagauggaggaggauauucug

SEQ ID NO: 168 - RNA, 21 nt, ARTIFICIAL SEQUENCE
  Synthetic Oligonucleotide
cagauggaggaggauauucua

SEQ ID NO: 169 - RNA, 21 nt, ARTIFICIAL SEQUENCE
  Synthetic Oligonucleotide
auggaggaggauauucugcag

SEQ ID NO: 170 - RNA, 21 nt, ARTIFICIAL SEQUENCE
  Synthetic Oligonucleotide
auggaggaggauauucugcaa

SEQ ID NO: 171 - RNA, 21 nt, ARTIFICIAL SEQUENCE
  Synthetic Oligonucleotide
auggaggaggcuauucugcag

SEQ ID NO: 172 - RNA, 21 nt, ARTIFICIAL SEQUENCE
  Synthetic Oligonucleotide
auggaggaggauauucugcag

SEQ ID NO: 173 - RNA, 21 nt, ARTIFICIAL SEQUENCE
  Synthetic Oligonucleotide
auggaggaggguauucugcag

SEQ ID NO: 174 - RNA, 23 nt, ARTIFICIAL SEQUENCE
  Synthetic Oligonucleotide
agauggaggaggauauucugcag

SEQ ID NO: 175 - RNA, 24 nt, ARTIFICIAL SEQUENCE
  Synthetic Oligonucleotide
cagauggaggaggauauucugcag

SEQ ID NO: 176 - RNA, 25 nt, ARTIFICIAL SEQUENCE
  Synthetic Oligonucleotide
ccagauggaggaggauauucugcag

SEQ ID NO: 177 - RNA, 21 nt, ARTIFICIAL SEQUENCE
  Synthetic Oligonucleotide
ggaggaggauauucugcagcu

SEQ ID NO: 178 - RNA, 21 nt, ARTIFICIAL SEQUENCE
  Synthetic Oligonucleotide
gcccaggcacagaaggugcua

SEQ ID NO: 179 - RNA, 21 nt, ARTIFICIAL SEQUENCE
  Synthetic Oligonucleotide
agcggcuagaaguccagcuga

SEQ ID NO: 180 - RNA, 21 nt, ARTIFICIAL SEQUENCE
  Synthetic Oligonucleotide
gcggcuagaaguccagcugaa

SEQ ID NO: 181 - RNA, 21 nt, ARTIFICIAL SEQUENCE
  Synthetic Oligonucleotide
cggcuagaaguccagcugaga

SEQ ID NO: 182 - RNA, 21 nt, ARTIFICIAL SEQUENCE
  Synthetic Oligonucleotide
ccgagaauuugaggucuuaaa

SEQ ID NO: 183 - RNA, 21 nt, ARTIFICIAL SEQUENCE
  Synthetic Oligonucleotide
cgagaauuugaggucuuaaag

SEQ ID NO: 184 - RNA, 21 nt, ARTIFICIAL SEQUENCE
  Synthetic Oligonucleotide
ggagaauuugaggucuuaaaa

SEQ ID NO: 185 - RNA, 21 nt, ARTIFICIAL SEQUENCE
  Synthetic Oligonucleotide
cgagaauuugaggucuuaaaa

SEQ ID NO: 186 - RNA, 21 nt, ARTIFICIAL SEQUENCE
  Synthetic Oligonucleotide
agaauuugaggucuuaaagga

SEQ ID NO: 187 - RNA, 21 nt, ARTIFICIAL SEQUENCE
  Synthetic Oligonucleotide
gaauuugaggucuuaaaggca

SEQ ID NO: 188 - RNA, 21 nt, ARTIFICIAL SEQUENCE
  Synthetic Oligonucleotide
aauuugaggucuuaaaggcua

SEQ ID NO: 189 - RNA, 21 nt, ARTIFICIAL SEQUENCE
  Synthetic Oligonucleotide
auuugaggucuuaaaggcuca

SEQ ID NO: 190 - RNA, 21 nt, ARTIFICIAL SEQUENCE
  Synthetic Oligonucleotide
aucugaggucuuaaaggcuca

SEQ ID NO: 191 - RNA, 21 nt, ARTIFICIAL SEQUENCE
  Synthetic Oligonucleotide
accugaggucuuaaaggcuca

SEQ ID NO: 192 - RNA, 21 nt, ARTIFICIAL SEQUENCE
  Synthetic Oligonucleotide
uuugaggucuuaaaggcucaa

SEQ ID NO: 193 - RNA, 21 nt, ARTIFICIAL SEQUENCE
  Synthetic Oligonucleotide
gaggucuuaaaggcucacgcu

SEQ ID NO: 194 - RNA, 21 nt, ARTIFICIAL SEQUENCE
  Synthetic Oligonucleotide
gaggucuuaaaggcucacgca

SEQ ID NO: 195 - RNA, 21 nt, ARTIFICIAL SEQUENCE
  Synthetic Oligonucleotide
ggucuuaaaggcucacgcuga

SEQ ID NO: 196 - RNA, 21 nt, ARTIFICIAL SEQUENCE
  Synthetic Oligonucleotide
ugucuuaaaggcucacgcugc

SEQ ID NO: 197 - RNA, 21 nt, ARTIFICIAL SEQUENCE
  Synthetic Oligonucleotide
gucuuaaaggcucacgcugaa

SEQ ID NO: 198 - RNA, 21 nt, ARTIFICIAL SEQUENCE
  Synthetic Oligonucleotide
ucuuaaaggcucacgcugaca

SEQ ID NO: 199 - RNA, 21 nt, ARTIFICIAL SEQUENCE
  Synthetic Oligonucleotide
ccgaaaggcucacgcugacaa

SEQ ID NO: 200 - RNA, 21 nt, ARTIFICIAL SEQUENCE
  Synthetic Oligonucleotide
cuuaaaggcucacgcugacaa

SEQ ID NO: 201 - RNA, 21 nt, ARTIFICIAL SEQUENCE
  Synthetic Oligonucleotide
cacaaaggcucacgcugacaa

SEQ ID NO: 202 - RNA, 21 nt, ARTIFICIAL SEQUENCE
  Synthetic Oligonucleotide
ccuaaaggcucacgcugacaa

SEQ ID NO: 203 - RNA, 21 nt, ARTIFICIAL SEQUENCE
  Synthetic Oligonucleotide
cugaaaggcucacgcugacaa

SEQ ID NO: 204 - RNA, 21 nt, ARTIFICIAL SEQUENCE
  Synthetic Oligonucleotide
cucaaaggcucacgcugacaa

SEQ ID NO: 205 - RNA, 21 nt, ARTIFICIAL SEQUENCE
  Synthetic Oligonucleotide
ccaaaaggcucacgcugacaa

SEQ ID NO: 206 - RNA, 21 nt, ARTIFICIAL SEQUENCE
  Synthetic Oligonucleotide
caaaaaggcucacgcugacaa

SEQ ID NO: 207 - RNA, 21 nt, ARTIFICIAL SEQUENCE
  Synthetic Oligonucleotide
cgcaaaggcucacgcugacaa

SEQ ID NO: 208 - RNA, 21 nt, ARTIFICIAL SEQUENCE
  Synthetic Oligonucleotide
cgaaaaggcucacgcugacaa

SEQ ID NO: 209 - RNA, 21 nt, ARTIFICIAL SEQUENCE
  Synthetic Oligonucleotide
cagaaaggcucacgcugacaa

SEQ ID NO: 210 - RNA, 21 nt, ARTIFICIAL SEQUENCE
  Synthetic Oligonucleotide
cccaaaggcucacgcugacaa

SEQ ID NO: 211 - RNA, 21 nt, ARTIFICIAL SEQUENCE
  Synthetic Oligonucleotide
cguaaaggcucacgcugacaa

SEQ ID NO: 212 - RNA, 21 nt, ARTIFICIAL SEQUENCE
  Synthetic Oligonucleotide
cggaaaggcucacgcugacaa

SEQ ID NO: 213 - RNA, 21 nt, ARTIFICIAL SEQUENCE
  Synthetic Oligonucleotide
uuaaaggcucacgcugacaaa

SEQ ID NO: 214 - RNA, 21 nt, ARTIFICIAL SEQUENCE
  Synthetic Oligonucleotide
aggcucacgcugacaagcaga

SEQ ID NO: 215 - RNA, 21 nt, ARTIFICIAL SEQUENCE
  Synthetic Oligonucleotide
ugcagcggcagaggcgggaga

SEQ ID NO: 216 - RNA, 21 nt, ARTIFICIAL SEQUENCE
  Synthetic Oligonucleotide
gcggcagaggcgggagauggu

SEQ ID NO: 217 - RNA, 21 nt, ARTIFICIAL SEQUENCE
  Synthetic Oligonucleotide
gaggcgggagaugguggcaca

SEQ ID NO: 218 - RNA, 21 nt, ARTIFICIAL SEQUENCE
  Synthetic Oligonucleotide
gcgggagaugguggcacagca

SEQ ID NO: 219 - RNA, 21 nt, ARTIFICIAL SEQUENCE
  Synthetic Oligonucleotide
guggcacagcagcaucggcua

SEQ ID NO: 220 - RNA, 21 nt, ARTIFICIAL SEQUENCE
  Synthetic Oligonucleotide
cagcagcaucggcugcgacaa

SEQ ID NO: 221 - RNA, 21 nt, ARTIFICIAL SEQUENCE
  Synthetic Oligonucleotide
agcagcaucggcugcgacaga

SEQ ID NO: 222 - RNA, 21 nt, ARTIFICIAL SEQUENCE
  Synthetic Oligonucleotide
gcagcaucggcugcgacagaa

SEQ ID NO: 223 - RNA, 21 nt, ARTIFICIAL SEQUENCE
  Synthetic Oligonucleotide
cagcaucggcugcgacagaua

SEQ ID NO: 224 - RNA, 21 nt, ARTIFICIAL SEQUENCE
  Synthetic Oligonucleotide
agcaucggcugcgacagauca

SEQ ID NO: 225 - RNA, 21 nt, ARTIFICIAL SEQUENCE
  Synthetic Oligonucleotide
gcaucggcugcgacagaucca

SEQ ID NO: 226 - RNA, 21 nt, ARTIFICIAL SEQUENCE
  Synthetic Oligonucleotide
caucggcugcgacagauccaa

SEQ ID NO: 227 - RNA, 21 nt, ARTIFICIAL SEQUENCE
  Synthetic Oligonucleotide
ucggcugcgacagauccagga

SEQ ID NO: 228 - RNA, 21 nt, ARTIFICIAL SEQUENCE
  Synthetic Oligonucleotide
ggcugcgacagauccaggaga

SEQ ID NO: 229 - RNA, 21 nt, ARTIFICIAL SEQUENCE
  Synthetic Oligonucleotide
gcugcgacagauccaggagaa

SEQ ID NO: 230 - RNA, 21 nt, ARTIFICIAL SEQUENCE
  Synthetic Oligonucleotide
cugcgacagauccaggagaga

SEQ ID NO: 231 - RNA, 23 nt, ARTIFICIAL SEQUENCE
  Synthetic Oligonucleotide
uggaagagcagggucagcuccuc

SEQ ID NO: 232 - RNA, 23 nt, ARTIFICIAL SEQUENCE
  Synthetic Oligonucleotide
uugucagccgucccuccgugguc

SEQ ID NO: 233 - RNA, 23 nt, ARTIFICIAL SEQUENCE
  Synthetic Oligonucleotide
uuccuggccuuugucagccgucc

SEQ ID NO: 234 - RNA, 23 nt, ARTIFICIAL SEQUENCE
  Synthetic Oligonucleotide
uguuccuggccuuugucagccgu

SEQ ID NO: 235 - RNA, 23 nt, ARTIFICIAL SEQUENCE
  Synthetic Oligonucleotide
uagagacccaggcuguuccuggc

SEQ ID NO: 236 - RNA, 23 nt, ARTIFICIAL SEQUENCE
  Synthetic Oligonucleotide
gagagacccaggcuguuccuggc

SEQ ID NO: 237 - RNA, 23 nt, ARTIFICIAL SEQUENCE
  Synthetic Oligonucleotide
auagagacccaggcuguuccugg

SEQ ID NO: 238 - RNA, 23 nt, ARTIFICIAL SEQUENCE
  Synthetic Oligonucleotide
uuagagacccaggcuguuccugg

SEQ ID NO: 239 - RNA, 23 nt, ARTIFICIAL SEQUENCE
  Synthetic Oligonucleotide
ugcggccauagagacccaggcug

SEQ ID NO: 240 - RNA, 23 nt, ARTIFICIAL SEQUENCE
  Synthetic Oligonucleotide
ugugcggccauagagacccaggc

SEQ ID NO: 241 - RNA, 23 nt, ARTIFICIAL SEQUENCE
  Synthetic Oligonucleotide
ugugcggccauagagaccaaggc

SEQ ID NO: 242 - RNA, 23 nt, ARTIFICIAL SEQUENCE
  Synthetic Oligonucleotide
ugugcggccauagagacccagug

SEQ ID NO: 243 - RNA, 23 nt, ARTIFICIAL SEQUENCE
  Synthetic Oligonucleotide
ugugcggccauagagacccagcc

SEQ ID NO: 244 - RNA, 23 nt, ARTIFICIAL SEQUENCE
  Synthetic Oligonucleotide
ugugcggccauagagacccaguu

SEQ ID NO: 245 - RNA, 23 nt, ARTIFICIAL SEQUENCE
  Synthetic Oligonucleotide
ugugcggccauagagacccagag

SEQ ID NO: 246 - RNA, 23 nt, ARTIFICIAL SEQUENCE
  Synthetic Oligonucleotide
ugugcggccauagagacccagau

SEQ ID NO: 247 - RNA, 23 nt, ARTIFICIAL SEQUENCE
  Synthetic Oligonucleotide
ugugcggccauagagacccaggu

SEQ ID NO: 248 - RNA, 23 nt, ARTIFICIAL SEQUENCE
  Synthetic Oligonucleotide
ugugcggccauagagacccagaa

SEQ ID NO: 249 - RNA, 23 nt, ARTIFICIAL SEQUENCE
  Synthetic Oligonucleotide
ugugcggccauagagaccuaggc

SEQ ID NO: 250 - RNA, 23 nt, ARTIFICIAL SEQUENCE
  Synthetic Oligonucleotide
ugugcggccauagagacccagcg

SEQ ID NO: 251 - RNA, 23 nt, ARTIFICIAL SEQUENCE
  Synthetic Oligonucleotide
ugugcggccauagagaccuuggc

SEQ ID NO: 252 - RNA, 23 nt, ARTIFICIAL SEQUENCE
  Synthetic Oligonucleotide
ugugcggccauagagacccagca

SEQ ID NO: 253 - RNA, 23 nt, ARTIFICIAL SEQUENCE
  Synthetic Oligonucleotide
ugugcggccauagagacccagac

SEQ ID NO: 254 - RNA, 23 nt, ARTIFICIAL SEQUENCE
  Synthetic Oligonucleotide
ugugcggccauagagacccagcu

SEQ ID NO: 255 - RNA, 23 nt, ARTIFICIAL SEQUENCE
  Synthetic Oligonucleotide
ugugcggccauagagaccggggc

SEQ ID NO: 256 - RNA, 23 nt, ARTIFICIAL SEQUENCE
  Synthetic Oligonucleotide
ugugcggccauagagacccagua

SEQ ID NO: 257 - RNA, 23 nt, ARTIFICIAL SEQUENCE
  Synthetic Oligonucleotide
ugugcggccauagagaccauggc

SEQ ID NO: 258 - RNA, 23 nt, ARTIFICIAL SEQUENCE
  Synthetic Oligonucleotide
ugugcggccauagagacccuggc

SEQ ID NO: 259 - RNA, 23 nt, ARTIFICIAL SEQUENCE
  Synthetic Oligonucleotide
ugugcggccauagagaccucggc

SEQ ID NO: 260 - RNA, 23 nt, ARTIFICIAL SEQUENCE
  Synthetic Oligonucleotide
ugugcggccauagagaccccggc

SEQ ID NO: 261 - RNA, 23 nt, ARTIFICIAL SEQUENCE
  Synthetic Oligonucleotide
ugugcggccauagagaccugggc

SEQ ID NO: 262 - RNA, 23 nt, ARTIFICIAL SEQUENCE
  Synthetic Oligonucleotide
aguuccugggcugcaucccggcc

SEQ ID NO: 263 - RNA, 23 nt, ARTIFICIAL SEQUENCE
  Synthetic Oligonucleotide
aaguuccugggcugcaucccggc

SEQ ID NO: 264 - RNA, 23 nt, ARTIFICIAL SEQUENCE
  Synthetic Oligonucleotide
uuugcccgaaguuccugggcugc

SEQ ID NO: 265 - RNA, 23 nt, ARTIFICIAL SEQUENCE
  Synthetic Oligonucleotide
ucuugcccgaaguuccugggcug

SEQ ID NO: 266 - RNA, 23 nt, ARTIFICIAL SEQUENCE
  Synthetic Oligonucleotide
uaggcuugcccgaaguuccuggg

SEQ ID NO: 267 - RNA, 23 nt, ARTIFICIAL SEQUENCE
  Synthetic Oligonucleotide
ucaggcuugcccgaaguuccugg

SEQ ID NO: 268 - RNA, 23 nt, ARTIFICIAL SEQUENCE
  Synthetic Oligonucleotide
ucaacaggcuugcccgaaguucc

SEQ ID NO: 269 - RNA, 23 nt, ARTIFICIAL SEQUENCE
  Synthetic Oligonucleotide
uccaacaggcuugcccgaaguuc

SEQ ID NO: 270 - RNA, 23 nt, ARTIFICIAL SEQUENCE
  Synthetic Oligonucleotide
ucuccaacaggcuugcccgaagu

SEQ ID NO: 271 - RNA, 23 nt, ARTIFICIAL SEQUENCE
  Synthetic Oligonucleotide
ugagucuccaacaggcuugcccg

SEQ ID NO: 272 - RNA, 23 nt, ARTIFICIAL SEQUENCE
  Synthetic Oligonucleotide
ucugagucuccaacaggcuugcc

SEQ ID NO: 273 - RNA, 23 nt, ARTIFICIAL SEQUENCE
  Synthetic Oligonucleotide
aucugagucuccaacaggcuugc

SEQ ID NO: 274 - RNA, 23 nt, ARTIFICIAL SEQUENCE
  Synthetic Oligonucleotide
uccaucugagucuccaacaggcu

SEQ ID NO: 275 - RNA, 23 nt, ARTIFICIAL SEQUENCE
  Synthetic Oligonucleotide
uuccaucugagucuccaacaggc

SEQ ID NO: 276 - RNA, 23 nt, ARTIFICIAL SEQUENCE
  Synthetic Oligonucleotide
uccuccuccaucugagucuccaa

SEQ ID NO: 277 - RNA, 23 nt, ARTIFICIAL SEQUENCE
  Synthetic Oligonucleotide
auccuccuccaucugagucucca

SEQ ID NO: 278 - RNA, 23 nt, ARTIFICIAL SEQUENCE
  Synthetic Oligonucleotide
uauccuccuccaucugagucucc

SEQ ID NO: 279 - RNA, 23 nt, ARTIFICIAL SEQUENCE
  Synthetic Oligonucleotide
auauccuccuccaucugagucuc

SEQ ID NO: 280 - RNA, 23 nt, ARTIFICIAL SEQUENCE
  Synthetic Oligonucleotide
aauauccuccuccaucugagucu

SEQ ID NO: 281 - RNA, 23 nt, ARTIFICIAL SEQUENCE
  Synthetic Oligonucleotide
uauauccuccuccaucugagucu

SEQ ID NO: 282 - RNA, 23 nt, ARTIFICIAL SEQUENCE
  Synthetic Oligonucleotide
agaauauccuccuccaucugagu

SEQ ID NO: 283 - RNA, 23 nt, ARTIFICIAL SEQUENCE
  Synthetic Oligonucleotide
ugaauauccuccuccaucugcgu

SEQ ID NO: 284 - RNA, 23 nt, ARTIFICIAL SEQUENCE
  Synthetic Oligonucleotide
ugaauauccuccuccaucugagu

SEQ ID NO: 285 - RNA, 23 nt, ARTIFICIAL SEQUENCE
  Synthetic Oligonucleotide
ggaauauccuccuccaucugagu

SEQ ID NO: 286 - RNA, 23 nt, ARTIFICIAL SEQUENCE
  Synthetic Oligonucleotide
cagaauauccuccuccaucugag

SEQ ID NO: 287 - RNA, 23 nt, ARTIFICIAL SEQUENCE
  Synthetic Oligonucleotide
uagaauauccuccuccaucugag

SEQ ID NO: 288 - RNA, 23 nt, ARTIFICIAL SEQUENCE
  Synthetic Oligonucleotide
cugcagaauauccuccuccaucu

SEQ ID NO: 289 - RNA, 23 nt, ARTIFICIAL SEQUENCE
  Synthetic Oligonucleotide
uugcagaauauccuccuccaucu

SEQ ID NO: 290 - DNA, 26 bp, ARTIFICIAL SEQUENCE
  Synthetic Oligonucleotide
cugcagaauauccuccuccaucuttt

SEQ ID NO: 291 - DNA, 25 bp, ARTIFICIAL SEQUENCE
  Synthetic Oligonucleotide
cugcagaauauccuccuccaucutt

SEQ ID NO: 292 - RNA, 23 nt, ARTIFICIAL SEQUENCE
  Synthetic Oligonucleotide
agcugcagaauauccuccuccau

SEQ ID NO: 293 - RNA, 23 nt, ARTIFICIAL SEQUENCE
  Synthetic Oligonucleotide
uagcaccuucugugccugggcca

SEQ ID NO: 294 - RNA, 23 nt, ARTIFICIAL SEQUENCE
  Synthetic Oligonucleotide
ucagcuggacuucuagccgcugc

SEQ ID NO: 295 - RNA, 23 nt, ARTIFICIAL SEQUENCE
  Synthetic Oligonucleotide
uucagcuggacuucuagccgcug

SEQ ID NO: 296 - RNA, 23 nt, ARTIFICIAL SEQUENCE
  Synthetic Oligonucleotide
ucucagcuggacuucuagccgcu

SEQ ID NO: 297 - RNA, 23 nt, ARTIFICIAL SEQUENCE
  Synthetic Oligonucleotide
uuuaagaccucaaauucucggua

SEQ ID NO: 298 - RNA, 23 nt, ARTIFICIAL SEQUENCE
  Synthetic Oligonucleotide
cuuuaagaccucaaauucucggu

SEQ ID NO: 299 - RNA, 23 nt, ARTIFICIAL SEQUENCE
  Synthetic Oligonucleotide
uuuuaagaccucaaauucuccgu

SEQ ID NO: 300 - RNA, 23 nt, ARTIFICIAL SEQUENCE
  Synthetic Oligonucleotide
uuuuaagaccucaaauucucggu

SEQ ID NO: 301 - RNA, 23 nt, ARTIFICIAL SEQUENCE
  Synthetic Oligonucleotide
uccuuuaagaccucaaauucucg

SEQ ID NO: 302 - RNA, 23 nt, ARTIFICIAL SEQUENCE
  Synthetic Oligonucleotide
ugccuuuaagaccucaaauucuc

SEQ ID NO: 303 - RNA, 23 nt, ARTIFICIAL SEQUENCE
  Synthetic Oligonucleotide
uagccuuuaagaccucaaauucu

SEQ ID NO: 304 - RNA, 23 nt, ARTIFICIAL SEQUENCE
  Synthetic Oligonucleotide
ugagccuuuaagaccucaaauuc

SEQ ID NO: 305 - RNA, 23 nt, ARTIFICIAL SEQUENCE
  Synthetic Oligonucleotide
ugagccuuuaagaccucagauuc

SEQ ID NO: 306 - RNA, 23 nt, ARTIFICIAL SEQUENCE
  Synthetic Oligonucleotide
ugagccuuuaagaccucagguuc

SEQ ID NO: 307 - RNA, 23 nt, ARTIFICIAL SEQUENCE
  Synthetic Oligonucleotide
uugagccuuuaagaccucaaauu

SEQ ID NO: 308 - RNA, 23 nt, ARTIFICIAL SEQUENCE
  Synthetic Oligonucleotide
agcgugagccuuuaagaccucaa

SEQ ID NO: 309 - RNA, 23 nt, ARTIFICIAL SEQUENCE
  Synthetic Oligonucleotide
ugcgugagccuuuaagaccucaa

SEQ ID NO: 310 - RNA, 23 nt, ARTIFICIAL SEQUENCE
  Synthetic Oligonucleotide
ucagcgugagccuuuaagaccuc

SEQ ID NO: 311 - RNA, 23 nt, ARTIFICIAL SEQUENCE
  Synthetic Oligonucleotide
gcagcgugagccuuuaagacauc

SEQ ID NO: 312 - RNA, 23 nt, ARTIFICIAL SEQUENCE
  Synthetic Oligonucleotide
uucagcgugagccuuuaagaccu

SEQ ID NO: 313 - RNA, 23 nt, ARTIFICIAL SEQUENCE
  Synthetic Oligonucleotide
ugucagcgugagccuuuaagacc

SEQ ID NO: 314 - RNA, 23 nt, ARTIFICIAL SEQUENCE
  Synthetic Oligonucleotide
uugucagcgugagccuuucggac

SEQ ID NO: 315 - RNA, 23 nt, ARTIFICIAL SEQUENCE
  Synthetic Oligonucleotide
uugucagcgugagccuuuaagaa

SEQ ID NO: 316 - RNA, 23 nt, ARTIFICIAL SEQUENCE
  Synthetic Oligonucleotide
uugucagcgugagccuuugugac

SEQ ID NO: 317 - RNA, 23 nt, ARTIFICIAL SEQUENCE
  Synthetic Oligonucleotide
uugucagcgugagccuuuaaggu

SEQ ID NO: 318 - RNA, 23 nt, ARTIFICIAL SEQUENCE
  Synthetic Oligonucleotide
uugucagcgugagccuuuaggac

SEQ ID NO: 319 - RNA, 23 nt, ARTIFICIAL SEQUENCE
  Synthetic Oligonucleotide
uugucagcgugagccuuuaagca

SEQ ID NO: 320 - RNA, 23 nt, ARTIFICIAL SEQUENCE
  Synthetic Oligonucleotide
uugucagcgugagccuuuaagag

SEQ ID NO: 321 - RNA, 23 nt, ARTIFICIAL SEQUENCE
  Synthetic Oligonucleotide
uugucagcgugagccuuucagac

SEQ ID NO: 322 - RNA, 23 nt, ARTIFICIAL SEQUENCE
  Synthetic Oligonucleotide
uugucagcgugagccuuuaaggg

SEQ ID NO: 323 - RNA, 23 nt, ARTIFICIAL SEQUENCE
  Synthetic Oligonucleotide
uugucagcgugagccuuugagac

SEQ ID NO: 324 - RNA, 23 nt, ARTIFICIAL SEQUENCE
  Synthetic Oligonucleotide
uugucagcgugagccuuuuggac

SEQ ID NO: 325 - RNA, 23 nt, ARTIFICIAL SEQUENCE
  Synthetic Oligonucleotide
uugucagcgugagccuuuaagua

SEQ ID NO: 326 - RNA, 23 nt, ARTIFICIAL SEQUENCE
  Synthetic Oligonucleotide
uugucagcgugagccuuuaaggc

SEQ ID NO: 327 - RNA, 23 nt, ARTIFICIAL SEQUENCE
  Synthetic Oligonucleotide
uugucagcgugagccuuuaagcu

SEQ ID NO: 328 - RNA, 23 nt, ARTIFICIAL SEQUENCE
  Synthetic Oligonucleotide
uugucagcgugagccuuuaagga

SEQ ID NO: 329 - RNA, 23 nt, ARTIFICIAL SEQUENCE
  Synthetic Oligonucleotide
uugucagcgugagccuuuaagug

SEQ ID NO: 330 - RNA, 23 nt, ARTIFICIAL SEQUENCE
  Synthetic Oligonucleotide
uugucagcgugagccuuuuugac

SEQ ID NO: 331 - RNA, 23 nt, ARTIFICIAL SEQUENCE
  Synthetic Oligonucleotide
uugucagcgugagccuuugcgac

SEQ ID NO: 332 - RNA, 23 nt, ARTIFICIAL SEQUENCE
  Synthetic Oligonucleotide
uugucagcgugagccuuuucgac

SEQ ID NO: 333 - RNA, 23 nt, ARTIFICIAL SEQUENCE
  Synthetic Oligonucleotide
uugucagcgugagccuuuaagcg

SEQ ID NO: 334 - RNA, 23 nt, ARTIFICIAL SEQUENCE
  Synthetic Oligonucleotide
uugucagcgugagccuuucugac

SEQ ID NO: 335 - RNA, 23 nt, ARTIFICIAL SEQUENCE
  Synthetic Oligonucleotide
uugucagcgugagccuuugggac

SEQ ID NO: 336 - RNA, 23 nt, ARTIFICIAL SEQUENCE
  Synthetic Oligonucleotide
uugucagcgugagccuuuaagac

SEQ ID NO: 337 - RNA, 23 nt, ARTIFICIAL SEQUENCE
  Synthetic Oligonucleotide
uugucagcgugagccuuuaagcc

SEQ ID NO: 338 - RNA, 23 nt, ARTIFICIAL SEQUENCE
  Synthetic Oligonucleotide
uugucagcgugagccuuuaagau

SEQ ID NO: 339 - RNA, 23 nt, ARTIFICIAL SEQUENCE
  Synthetic Oligonucleotide
uugucagcgugagccuuuacgac

SEQ ID NO: 340 - RNA, 23 nt, ARTIFICIAL SEQUENCE
  Synthetic Oligonucleotide
uugucagcgugagccuuuaaguc

SEQ ID NO: 341 - RNA, 23 nt, ARTIFICIAL SEQUENCE
  Synthetic Oligonucleotide
uugucagcgugagccuuuaaguu

SEQ ID NO: 342 - RNA, 23 nt, ARTIFICIAL SEQUENCE
  Synthetic Oligonucleotide
uugucagcgugagccuuuccgac

SEQ ID NO: 343 - RNA, 23 nt, ARTIFICIAL SEQUENCE
  Synthetic Oligonucleotide
uuugucagcgugagccuuuaaga

SEQ ID NO: 344 - RNA, 23 nt, ARTIFICIAL SEQUENCE
  Synthetic Oligonucleotide
ucugcuugucagcgugagccuuu

SEQ ID NO: 345 - RNA, 23 nt, ARTIFICIAL SEQUENCE
  Synthetic Oligonucleotide
ucucccgccucugccgcugcacg

SEQ ID NO: 346 - RNA, 23 nt, ARTIFICIAL SEQUENCE
  Synthetic Oligonucleotide
accaucucccgccucugccgcug

SEQ ID NO: 347 - RNA, 23 nt, ARTIFICIAL SEQUENCE
  Synthetic Oligonucleotide
ugugccaccaucucccgccucug

SEQ ID NO: 348 - RNA, 23 nt, ARTIFICIAL SEQUENCE
  Synthetic Oligonucleotide
ugcugugccaccaucucccgccu

SEQ ID NO: 349 - RNA, 23 nt, ARTIFICIAL SEQUENCE
  Synthetic Oligonucleotide
uagccgaugcugcugugccacca

SEQ ID NO: 350 - RNA, 23 nt, ARTIFICIAL SEQUENCE
  Synthetic Oligonucleotide
uugucgcagccgaugcugcugug

SEQ ID NO: 351 - RNA, 23 nt, ARTIFICIAL SEQUENCE
  Synthetic Oligonucleotide
ucugucgcagccgaugcugcugu

SEQ ID NO: 352 - RNA, 23 nt, ARTIFICIAL SEQUENCE
  Synthetic Oligonucleotide
uucugucgcagccgaugcugcug

SEQ ID NO: 353 - RNA, 23 nt, ARTIFICIAL SEQUENCE
  Synthetic Oligonucleotide
uaucugucgcagccgaugcugcu

SEQ ID NO: 354 - RNA, 23 nt, ARTIFICIAL SEQUENCE
  Synthetic Oligonucleotide
ugaucugucgcagccgaugcugc

SEQ ID NO: 355 - RNA, 23 nt, ARTIFICIAL SEQUENCE
  Synthetic Oligonucleotide
uggaucugucgcagccgaugcug

SEQ ID NO: 356 - RNA, 23 nt, ARTIFICIAL SEQUENCE
  Synthetic Oligonucleotide
uuggaucugucgcagccgaugcu

SEQ ID NO: 357 - RNA, 23 nt, ARTIFICIAL SEQUENCE
  Synthetic Oligonucleotide
uccuggaucugucgcagccgaug

SEQ ID NO: 358 - RNA, 23 nt, ARTIFICIAL SEQUENCE
  Synthetic Oligonucleotide
ucuccuggaucugucgcagccga

SEQ ID NO: 359 - RNA, 23 nt, ARTIFICIAL SEQUENCE
  Synthetic Oligonucleotide
uucuccuggaucugucgcagccg

SEQ ID NO: 360 - RNA, 23 nt, ARTIFICIAL SEQUENCE
  Synthetic Oligonucleotide
ucucuccuggaucugucgcagcc

SEQ ID NO: 361 - RNA, 21 nt, ARTIFICIAL SEQUENCE
  Synthetic Oligonucleotide; sequence (5′ to 3′) with modifications mA*mU*mGmGmAmGfGmAfGfGfAmUmAmUmUmCmUmGmC*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
auggaggaggauauucugcaa

SEQ ID NO: 362 - RNA, 21 nt, ARTIFICIAL SEQUENCE
  Synthetic Oligonucleotide; sequence (5′ to 3′) with modifications mC*mC*mGmAmGmAfAmUfUfUfGmAmGmGmUmCmUmUmA*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
ccgagaauuugaggucuuaaa

SEQ ID NO: 363 - RNA, 21 nt, ARTIFICIAL SEQUENCE
  Synthetic Oligonucleotide; sequence (5′ to 3′) with modifications mA*mU*mGmGmAmGfGmAfGfGfAmUmAmUmUmCmUmGmC*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
auggaggaggauauucugcaa

SEQ ID NO: 364 - RNA, 21 nt, ARTIFICIAL SEQUENCE
  Synthetic Oligonucleotide; sequence (5′ to 3′) with modifications mG*mG*mUmCmUmUfAmAfAfGfGmCmUmCmAmCmGmCmU*mG*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
ggucuuaaaggcucacgcuga

SEQ ID NO: 365 - RNA, 21 nt, ARTIFICIAL SEQUENCE
  Synthetic Oligonucleotide; sequence (5′ to 3′) with modifications mA*mU*mGmGmAmGfGmAfGfGfAmUmAmUmUmCmUmGmC*mA*mG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
auggaggaggauauucugcag

SEQ ID NO: 366 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmA*mA*mG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
cgagaauuugaggucuuaaag

SEQ ID NO: 367 - RNA, 23 nt, ARTIFICIAL SEQUENCE
  Synthetic Oligonucleotide; sequence (5′ to 3′) with modifications [Phos]mU*fU*mGmCmAfGmAmAmUmAmUmCmCfUmCfCmUmCmCmAmU*mC*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uugcagaauauccuccuccaucu

SEQ ID NO: 368 - RNA, 23 nt, ARTIFICIAL SEQUENCE
  Synthetic Oligonucleotide; sequence (5′ to 3′) with modifications [Phos]mU*fU*mUmAmAfGmAmCmCmUmCmAmAfAmUfUmCmUmCmGmG*mU*mA
  misc_feature (1)..(1): 5′-phopshate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uuuaagaccucaaauucucggua

SEQ ID NO: 369 - RNA, 23 nt, ARTIFICIAL SEQUENCE
  Synthetic Oligonucleotide; sequence (5′ to 3′) with modifications mU*fU*mGmCmAfGmAmAmUmAmUmCmCfUmCfCmUmCmCmAmU*mC*mU
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uugcagaauauccuccuccaucu

SEQ ID NO: 370 - RNA, 23 nt, ARTIFICIAL SEQUENCE
  Synthetic Oligonucleotide; sequence (5′ to 3′) with modifications mU*fC*mAmGmCfGmUmGmAmGmCmCmUfUmUfAmAmGmAmCmC*mU*mC
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
ucagcgugagccuuuaagaccuc

SEQ ID NO: 371 - RNA, 23 nt, ARTIFICIAL SEQUENCE
  Synthetic Oligonucleotide; sequence (5′ to 3′) with modifications mC*fU*mGmCmAfGmAmAmUmAmUmCmCfUmCfCmUmCmCmAmU*mC*mU
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cugcagaauauccuccuccaucu

SEQ ID NO: 372 - RNA, 23 nt, ARTIFICIAL SEQUENCE
  Synthetic Oligonucleotide; sequence (5′ to 3′) with modifications mC*fU*mUmUmAfAmGmAmCmCmUmCmAfAmAfUmUmCmUmCmG*mG*mU
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 373 - RNA, 21 nt, ARTIFICIAL SEQUENCE
  Synthetic Oligonucleotide; sequence (5′ to 3′) with modifications mU*mC*mAmGmAmUmGmGfAfGfGmAmGmGmAmUmAmUmUmCmU
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
ucagauggaggaggauauucu

SEQ ID NO: 374 - RNA, 21 nt, ARTIFICIAL SEQUENCE
  Synthetic Oligonucleotide; sequence (5′ to 3′) with modifications mG*mC*mAmGmAmUfGmGfAfGfGmAmGmGmAmUmAmUmU*mC*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
gcagauggaggaggauauuca

SEQ ID NO: 375 - RNA, 21 nt, ARTIFICIAL SEQUENCE
  Synthetic Oligonucleotide; sequence (5′ to 3′) with modifications mU*mC*mAmGmAmUfGmGfAfGfGmAmGmGmAmUmAmUmUmCmU
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
ucagauggaggaggauauucu

SEQ ID NO: 376 - RNA, 21 nt, ARTIFICIAL SEQUENCE
  Synthetic Oligonucleotide; sequence (5′ to 3′) with modifications mU*mC*mAmGmAmUfGmGfAfGfGmAmGmGmAmUmAmUmU*mC*mC
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
ucagauggaggaggauauucc

SEQ ID NO: 377 - RNA, 21 nt, ARTIFICIAL SEQUENCE
  Synthetic Oligonucleotide; sequence (5′ to 3′) with modifications mU*mC*mAmGmAmUfGmGfAfGfGmAmGmGmAmUmAmUmUmCmUP
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
ucagauggaggaggauauucu

SEQ ID NO: 378 - RNA, 21 nt, ARTIFICIAL SEQUENCE
  Synthetic Oligonucleotide; sequence (5′ to 3′) with modifications mC*mA*mGmAmUmGfGmAfGfGfAmGmGmAmUmAmUmUmC*mU*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
cagauggaggaggauauucua

SEQ ID NO: 379 - RNA, 21 nt, ARTIFICIAL SEQUENCE
  Synthetic Oligonucleotide; sequence (5′ to 3′) with modifications mA*mU*mGmGmAmGfGmAfGfGfAmUmAmUmUmCmUmGmC*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
auggaggaggauauucugcaa

SEQ ID NO: 380 - RNA, 21 nt, ARTIFICIAL SEQUENCE
  Synthetic Oligonucleotide; sequence (5′ to 3′) with modifications mC*mC*mGmAmGmAfAmUfUfUfGmAmGmGmUmCmUmUmA*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
ccgagaauuugaggucuuaaa

SEQ ID NO: 381 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmA*mA*mG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
cgagaauuugaggucuuaaag

SEQ ID NO: 382 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmA*mA*mG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
cgagaauuugaggucuuaaag

SEQ ID NO: 383 - RNA, 21 nt, ARTIFICIAL SEQUENCE
  Synthetic Oligonucleotide; sequence (5′ to 3′) with modifications mG*mG*mAmGmAmAfUmUfUfGfAmGmGmUmCmUmUmAmA*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
ggagaauuugaggucuuaaaa

SEQ ID NO: 384 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmA*mA*mA
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide with a 3′-phosphorothioate linkage
  misc_feature (20)..(20): 2′-OMe modified nucleotide with a 3′-phosphorothioate linkage
  misc_feature (21)..(21): 2′-OMe modified nucleotide
cgagaauuugaggucuuaaaa

SEQ ID NO: 385 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmAmAmG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
cgagaauuugaggucuuaaag

SEQ ID NO: 386 - RNA, 21 nt, ARTIFICIAL SEQUENCE
  Synthetic Oligonucleotide; sequence (5′ to 3′) with modifications mC*mG*mAmGmAmAmUmUfUfGfAmGmGmUmCmUmUmAmAmAmG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
cgagaauuugaggucuuaaag

SEQ ID NO: 387 - RNA, 21 nt, ARTIFICIAL SEQUENCE
  Synthetic Oligonucleotide; sequence (5′ to 3′) with modifications mC*mG*mAmGmAmAfUmUfUfGfAmGmGmUmCmUmUmAmAmAmG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-F modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
cgagaauuugaggucuuaaag

SEQ ID NO: 388 - RNA, 21 nt, ARTIFICIAL SEQUENCE
  Synthetic Oligonucleotide; sequence (5′ to 3′) with modifications mC*mG*mAmGmAmAmUmUfUfGfAmGmGmUmCmUmUmAmAmAmG
  misc_feature (1)..(1): 2′-OMe modified nucleotide with a 3′-phosphorothioate linkage
  misc_feature (2)..(2): 2′-OMe modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-F modified nucleotide
  misc_feature (10)..(10): 2′-F modified nucleotide
  misc_feature (11)..(11): 2′-F modified nucleotide
  misc_feature (12)..(12): 2′-OMe modified nucleotide
  misc_feature (13)..(13): 2′-OMe modified nucleotide
  misc_feature (14)..(14): 2′-OMe modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide
cgagaauuugaggucuuaaag

SEQ ID NO: 389 - RNA, 23 nt, ARTIFICIAL SEQUENCE
  Synthetic Oligonucleotide; sequence (5′ to 3′) with modifications PmA*fG*mAmAmUfAmUmCmCmUmCmCmUfCmCfAmUmCmUmGmA*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
agaauauccuccuccaucugagu

SEQ ID NO: 390 - RNA, 23 nt, ARTIFICIAL SEQUENCE
  Synthetic Oligonucleotide; sequence (5′ to 3′) with modifications PmU*fG*mAmAmUfAmUmCmCmUmCmCmUfCmCfAmUmCmUmGmC*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
ugaauauccuccuccaucugcgu

SEQ ID NO: 391 - RNA, 23 nt, ARTIFICIAL SEQUENCE
  Synthetic Oligonucleotide; sequence (5′ to 3′) with modifications PmA*fG*mAmAmUfAmUmCmCmUmCmCmUfCmCfAmUmCmUmGmA*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
agaauauccuccuccaucugagu

SEQ ID NO: 392 - RNA, 23 nt, ARTIFICIAL SEQUENCE
  Synthetic Oligonucleotide; sequence (5′ to 3′) with modifications PmG*fG*mAmAmUfAmUmCmCmUmCmCmUfCmCfAmUmCmUmGmA*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
ggaauauccuccuccaucugagu

SEQ ID NO: 393 - RNA, 23 nt, ARTIFICIAL SEQUENCE
  Synthetic Oligonucleotide; sequence (5′ to 3′) with modifications PmA*fG*mAmAmUmAmUmCmCmUmCmCmUfCmCmAmUmCmUmGmA*mG*mU
  misc_feature (1)..(1): 5′-phopphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
agaauauccuccuccaucugagu

SEQ ID NO: 394 - RNA, 23 nt, ARTIFICIAL SEQUENCE
  Synthetic Oligonucleotide; sequence (5′ to 3′) with modifications PmU*fA*mGmAmAfUmAmUmCmCmUmCmCfUmCfCmAmUmCmUmG*mA*mG
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uagaauauccuccuccaucugag

SEQ ID NO: 395 - RNA, 23 nt, ARTIFICIAL SEQUENCE
  Synthetic Oligonucleotide; sequence (5′ to 3′) with modifications PmU*fU*mGmCmAfGmAmAmUmAmUmCmCfUmCfCmUmCmCmAmU*mC*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uugcagaauauccuccuccaucu

SEQ ID NO: 396 - RNA, 23 nt, ARTIFICIAL SEQUENCE
  Synthetic Oligonucleotide; sequence (5′ to 3′) with modifications PmU*fU*mUmAmAfGmAmCmCmUmCmAmAfAmUfUmCmUmCmGmG*mU*mA
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uuuaagaccucaaauucucggua

SEQ ID NO: 397 - RNA, 23 nt, ARTIFICIAL SEQUENCE
  Synthetic Oligonucleotide; sequence (5′ to 3′) with modifications PmC*fU*mUmUmAfAmGmAmCmCmUmCmAfAmAfUmUmCmUmCmG*mG*mU
  misc_feature (1)..(1): 5′-Phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 398 - RNA, 23 nt, ARTIFICIAL SEQUENCE
  Synthetic Oligonucleotide; sequence (5′ to 3′) with modifications PmC*fU*mUmUmAfAmGmAmCmCmUmCmAfAmAfUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 399 - RNA, 23 nt, ARTIFICIAL SEQUENCE
  Synthetic Oligonucleotide; sequence (5′ to 3′) with modifications PmU*fU*mUmUmAfAmGmAmCmCmUmCmAfAmAfUmUmCmUmCmC*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uuuuaagaccucaaauucuccgu

SEQ ID NO: 400 - RNA, 23 nt, ARTIFICIAL SEQUENCE
  Synthetic Oligonucleotide; sequence (5′ to 3′) with modifications PmU*fU*mUmUmAfAmGmAmCmCmUmCmAfAmAfUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
uuuuaagaccucaaauucucggu

SEQ ID NO: 401 - RNA, 23 nt, ARTIFICIAL SEQUENCE
  Synthetic Oligonucleotide; sequence (5′ to 3′) with modifications PmC*fU*mUmUmAfAmGmAmCmCmUmCmAfAmAfUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 402 - RNA, 23 nt, ARTIFICIAL SEQUENCE
  Synthetic Oligonucleotide; sequence (5′ to 3′) with modifications PmC*fU*mUmUmAfAmGmAmCmCmUmCmAfAmAfUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-F modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-F modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 403 - RNA, 23 nt, ARTIFICIAL SEQUENCE
  Synthetic Oligonucleotide; sequence (5′ to 3′) with modifications PmC*fU*mUmUmAmAmGmAmCmCmUmCmAfAmAmUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 404 - RNA, 23 nt, ARTIFICIAL SEQUENCE
  Synthetic Oligonucleotide; sequence (5′ to 3′) with modifications PmC*fU*mUmUmAmAmGmAmCmCmUmCmAfAmAmUmUmCmUmCmG*mG*mU
  misc_feature (1)..(1): 5′-phosphate
  misc_feature (1)..(1): 2′-OMe modified nucleotide with a 3′-phosphorothioate linkage
  misc_feature (2)..(2): 2′-F modified nucleotide with a 3′-phosphorothioate linkage
  misc_feature (3)..(3): 2′-OMe modified nucleotide
  misc_feature (4)..(4): 2′-OMe modified nucleotide
  misc_feature (5)..(5): 2′-OMe modified nucleotide
  misc_feature (6)..(6): 2′-OMe modified nucleotide
  misc_feature (7)..(7): 2′-OMe modified nucleotide
  misc_feature (8)..(8): 2′-OMe modified nucleotide
  misc_feature (9)..(9): 2′-OMe modified nucleotide
  misc_feature (10)..(10): 2′-OMe modified nucleotide
  misc_feature (11)..(11): 2′-OMe modified nucleotide
  misc_feature (12)..(12): 2′-OMe modified nucleotide
  misc_feature (13)..(13): 2′-OMe modified nucleotide
  misc_feature (14)..(14): 2′-F modified nucleotide
  misc_feature (15)..(15): 2′-OMe modified nucleotide
  misc_feature (16)..(16): 2′-OMe modified nucleotide
  misc_feature (17)..(17): 2′-OMe modified nucleotide
  misc_feature (18)..(18): 2′-OMe modified nucleotide
  misc_feature (19)..(19): 2′-OMe modified nucleotide
  misc_feature (20)..(20): 2′-OMe modified nucleotide
  misc_feature (21)..(21): 2′-OMe modified nucleotide with a 3′-phosphorothioate linkage
  misc_feature (22)..(22): 2′-OMe modified nucleotide with a 3′-phosphorothioate linkage
  misc_feature (23)..(23): 2′-OMe modified nucleotide
cuuuaagaccucaaauucucggu

SEQ ID NO: 405 - RNA, 18 nt, Homo sapiens
ggaagagcagggucagcu

SEQ ID NO: 406 - RNA, 18 nt, Homo sapiens
ggcccagcugcagggucc

SEQ ID NO: 407 - RNA, 18 nt, Homo sapiens
ugagggccuggcccagcu

SEQ ID NO: 408 - RNA, 18 nt, Homo sapiens
ugucagccgucccuccgu

SEQ ID NO: 409 - RNA, 18 nt, Homo sapiens
uugucagccgucccuccg

SEQ ID NO: 410 - RNA, 18 nt, Homo sapiens
ggccuuugucagccgucc

SEQ ID NO: 411 - RNA, 18 nt, Homo sapiens
ccuggccuuugucagccg

SEQ ID NO: 412 - RNA, 18 nt, Homo sapiens
uccuggccuuugucagcc

SEQ ID NO: 413 - RNA, 18 nt, Homo sapiens
guuccuggccuuugucag

SEQ ID NO: 414 - RNA, 18 nt, Homo sapiens
agagacccaggcuguucc

SEQ ID NO: 415 - RNA, 18 nt, Homo sapiens
uagagacccaggcuguuc

SEQ ID NO: 416 - RNA, 18 nt, Homo sapiens
gccauagagacccaggcu

SEQ ID NO: 417 - RNA, 18 nt, Homo sapiens
cggccauagagacccagg

SEQ ID NO: 418 - RNA, 18 nt, Homo sapiens
gcggccauagagacccag

SEQ ID NO: 419 - RNA, 18 nt, Homo sapiens
ugcggccauagagaccca

SEQ ID NO: 420 - RNA, 18 nt, Homo sapiens
gugcggccauagagaccc

SEQ ID NO: 421 - RNA, 18 nt, Homo sapiens
ucccggccccggcugacc

SEQ ID NO: 422 - RNA, 18 nt, Homo sapiens
gggcugcaucccggcccc

SEQ ID NO: 423 - RNA, 18 nt, Homo sapiens
ccugggcugcaucccggc

SEQ ID NO: 424 - RNA, 18 nt, Homo sapiens
uccugggcugcaucccgg

SEQ ID NO: 425 - RNA, 18 nt, Homo sapiens
guuccugggcugcauccc

SEQ ID NO: 426 - RNA, 18 nt, Homo sapiens
aguuccugggcugcaucc

SEQ ID NO: 427 - RNA, 18 nt, Homo sapiens
ugcccgaaguuccugggc

SEQ ID NO: 428 - RNA, 18 nt, Homo sapiens
uugcccgaaguuccuggg

SEQ ID NO: 429 - RNA, 18 nt, Homo sapiens
cuugcccgaaguuccugg

SEQ ID NO: 430 - RNA, 18 nt, Homo sapiens
gcuugcccgaaguuccug

SEQ ID NO: 431 - RNA, 18 nt, Homo sapiens
ggcuugcccgaaguuccu

SEQ ID NO: 432 - RNA, 18 nt, Homo sapiens
aggcuugcccgaaguucc

SEQ ID NO: 433 - RNA, 18 nt, Homo sapiens
caggcuugcccgaaguuc

SEQ ID NO: 434 - RNA, 18 nt, Homo sapiens
caacaggcuugcccgaag

SEQ ID NO: 435 - RNA, 18 nt, Homo sapiens
ccaacaggcuugcccgaa

SEQ ID NO: 436 - RNA, 18 nt, Homo sapiens
cuccaacaggcuugcccg

SEQ ID NO: 437 - RNA, 18 nt, Homo sapiens
gagucuccaacaggcuug

SEQ ID NO: 438 - RNA, 18 nt, Homo sapiens
cugagucuccaacaggcu

SEQ ID NO: 439 - RNA, 18 nt, Homo sapiens
ucugagucuccaacaggc

SEQ ID NO: 440 - RNA, 18 nt, Homo sapiens
ccaucugagucuccaaca

SEQ ID NO: 441 - RNA, 18 nt, Homo sapiens
uccaucugagucuccaac

SEQ ID NO: 442 - RNA, 18 nt, Homo sapiens
ccuccuccaucugagucu

SEQ ID NO: 443 - RNA, 18 nt, Homo sapiens
uccuccuccaucugaguc

SEQ ID NO: 444 - RNA, 18 nt, Homo sapiens
auccuccuccaucugagu

SEQ ID NO: 445 - RNA, 18 nt, Homo sapiens
uauccuccuccaucugag

SEQ ID NO: 446 - RNA, 18 nt, Homo sapiens
auauccuccuccaucuga

SEQ ID NO: 447 - RNA, 18 nt, Homo sapiens
aauauccuccuccaucug

SEQ ID NO: 448 - RNA, 18 nt, Homo sapiens
gaauauccuccuccaucu

SEQ ID NO: 449 - RNA, 18 nt, Homo sapiens
agaauauccuccuccauc

SEQ ID NO: 450 - RNA, 18 nt, Homo sapiens
cagaauauccuccuccau

SEQ ID NO: 451 - RNA, 18 nt, Homo sapiens
ugcagaauauccuccucc

SEQ ID NO: 452 - RNA, 18 nt, Homo sapiens
gcugcagaauauccuccu

SEQ ID NO: 453 - RNA, 18 nt, Homo sapiens
ucugugccugggccaccu

SEQ ID NO: 454 - RNA, 18 nt, Homo sapiens
agcaccuucugugccugg

SEQ ID NO: 455 - RNA, 18 nt, Homo sapiens
gcuggacuucuagccgcu

SEQ ID NO: 456 - RNA, 18 nt, Homo sapiens
agcuggacuucuagccgc

SEQ ID NO: 457 - RNA, 18 nt, Homo sapiens
cagcuggacuucuagccg

SEQ ID NO: 458 - RNA, 18 nt, Homo sapiens
ucagcuggacuucuagcc

SEQ ID NO: 459 - RNA, 18 nt, Homo sapiens
cucagcuggacuucuagc

SEQ ID NO: 460 - RNA, 18 nt, Homo sapiens
cgcuccucagcuggacuu

SEQ ID NO: 461 - RNA, 18 nt, Homo sapiens
gcgcuccucagcuggacu

SEQ ID NO: 462 - RNA, 18 nt, Homo sapiens
aggcagggcccagccagg

SEQ ID NO: 463 - RNA, 18 nt, Homo sapiens
uuaagaccucaaauucuc

SEQ ID NO: 464 - RNA, 18 nt, Homo sapiens
uuuaagaccucaaauucu

SEQ ID NO: 465 - RNA, 18 nt, Homo sapiens
ccuuuaagaccucaaauu

SEQ ID NO: 466 - RNA, 18 nt, Homo sapiens
gccuuuaagaccucaaau

SEQ ID NO: 467 - RNA, 18 nt, Homo sapiens
agccuuuaagaccucaaa

SEQ ID NO: 468 - RNA, 18 nt, Homo sapiens
gagccuuuaagaccucaa

SEQ ID NO: 469 - RNA, 18 nt, Homo sapiens
ugagccuuuaagaccuca

SEQ ID NO: 470 - RNA, 18 nt, Homo sapiens
gugagccuuuaagaccuc

SEQ ID NO: 471 - RNA, 18 nt, Homo sapiens
gcgugagccuuuaagacc

SEQ ID NO: 472 - RNA, 18 nt, Homo sapiens
cagcgugagccuuuaaga

SEQ ID NO: 473 - RNA, 18 nt, Homo sapiens
ucagcgugagccuuuaag

SEQ ID NO: 474 - RNA, 18 nt, Homo sapiens
gucagcgugagccuuuaa

SEQ ID NO: 475 - RNA, 18 nt, Homo sapiens
ugucagcgugagccuuua

SEQ ID NO: 476 - RNA, 18 nt, Homo sapiens
uugucagcgugagccuuu

SEQ ID NO: 477 - RNA, 18 nt, Homo sapiens
cuugucagcgugagccuu

SEQ ID NO: 478 - RNA, 18 nt, Homo sapiens
gcuugucagcgugagccu

SEQ ID NO: 479 - RNA, 18 nt, Homo sapiens
cugcuugucagcgugagc

SEQ ID NO: 480 - RNA, 18 nt, Homo sapiens
ucugcuugucagcgugag

SEQ ID NO: 481 - RNA, 18 nt, Homo sapiens
cucugcuugucagcguga

SEQ ID NO: 482 - RNA, 18 nt, Homo sapiens
gcucugcuugucagcgug

SEQ ID NO: 483 - RNA, 18 nt, Homo sapiens
ggcucugcuugucagcgu

SEQ ID NO: 484 - RNA, 18 nt, Homo sapiens
guggcucugcuugucagc

SEQ ID NO: 485 - RNA, 18 nt, Homo sapiens
gcacguggccugugaggg

SEQ ID NO: 486 - RNA, 18 nt, Homo sapiens
ugcacguggccugugagg

SEQ ID NO: 487 - RNA, 18 nt, Homo sapiens
ugccgcugcacguggccu

SEQ ID NO: 488 - RNA, 18 nt, Homo sapiens
cugccgcugcacguggcc

SEQ ID NO: 489 - RNA, 18 nt, Homo sapiens
ccucugccgcugcacgug

SEQ ID NO: 490 - RNA, 18 nt, Homo sapiens
cucccgccucugccgcug

SEQ ID NO: 491 - RNA, 18 nt, Homo sapiens
ccaucucccgccucugcc

SEQ ID NO: 492 - RNA, 18 nt, Homo sapiens
gugccaccaucucccgcc

SEQ ID NO: 493 - RNA, 18 nt, Homo sapiens
gcugugccaccaucuccc

SEQ ID NO: 494 - RNA, 18 nt, Homo sapiens
gcugcugugccaccaucu

SEQ ID NO: 495 - RNA, 18 nt, Homo sapiens
agccgaugcugcugugcc

SEQ ID NO: 496 - RNA, 18 nt, Homo sapiens
gcagccgaugcugcugug

SEQ ID NO: 497 - RNA, 18 nt, Homo sapiens
ucgcagccgaugcugcug

SEQ ID NO: 498 - RNA, 18 nt, Homo sapiens
gucgcagccgaugcugcu

SEQ ID NO: 499 - RNA, 18 nt, Homo sapiens
ugucgcagccgaugcugc

SEQ ID NO: 500 - RNA, 18 nt, Homo sapiens
cugucgcagccgaugcug

SEQ ID NO: 501 - RNA, 18 nt, Homo sapiens
ucugucgcagccgaugcu

SEQ ID NO: 502 - RNA, 18 nt, Homo sapiens
aucugucgcagccgaugc

SEQ ID NO: 503 - RNA, 18 nt, Homo sapiens
gaucugucgcagccgaug

SEQ ID NO: 504 - RNA, 18 nt, Homo sapiens
ggaucugucgcagccgau

SEQ ID NO: 505 - RNA, 18 nt, Homo sapiens
uggaucugucgcagccga

SEQ ID NO: 506 - RNA, 18 nt, Homo sapiens
ccuggaucugucgcagcc

SEQ ID NO: 507 - RNA, 18 nt, Homo sapiens
cuccuggaucugucgcag

SEQ ID NO: 508 - RNA, 18 nt, Homo sapiens
ucuccuggaucugucgca

SEQ ID NO: 509 - RNA, 18 nt, Homo sapiens
cucuccuggaucugucgc

SEQ ID NO: 510 - RNA, 18 nt, Homo sapiens
aggcagauucaggcuggg

SEQ ID NO: 511 - RNA, 18 nt, Homo sapiens
uccaggcagauucaggcu

SEQ ID NO: 512 - RNA, 18 nt, Homo sapiens
auccaggcagauucaggc

SEQ ID NO: 513 - RNA, 18 nt, Homo sapiens
ucaguuccauccaggcag

SEQ ID NO: 514 - RNA, 18 nt, Homo sapiens
guguuccuugcagcauga

SEQ ID NO: 515 - RNA, 18 nt, Homo sapiens
gcguggaaguguuccuug

SEQ ID NO: 516 - RNA, 18 nt, Homo sapiens
ggggcguggaaguguucc

SEQ ID NO: 517 - RNA, 18 nt, Homo sapiens
gaucccagugaacaggca

SEQ ID NO: 518 - RNA, 18 nt, Homo sapiens
ugaucccagugaacaggc

SEQ ID NO: 519 - RNA, 18 nt, Homo sapiens
gcugaucccagugaacag

SEQ ID NO: 520 - RNA, 18 nt, Homo sapiens
ggcugaucccagugaaca

SEQ ID NO: 521 - RNA, 18 nt, Homo sapiens
gcgcccuggcugauccca

SEQ ID NO: 522 - RNA, 18 nt, Homo sapiens
ggcgcccuggcugauccc

SEQ ID NO: 523 - RNA, 18 nt, Homo sapiens
cacgacucugcuuuaaug

SEQ ID NO: 524 - RNA, 18 nt, Homo sapiens
ccacgacucugcuuuaau

SEQ ID NO: 525 - RNA, 18 nt, Homo sapiens
ugccacgacucugcuuua